(19) United States
(12) Patent Application Publication
(10) Pub. No.: US 2009/0204336 A1
(43) Pub. Date: Aug. 13, 2009

(54) PDZ-Domain Modulators

(75) Inventors: Mangesh Joshi, Berlin (DE); Hartmut Oschkinat, Berlin (DE); Markus Schade, Berlin (DE); Carolyn Vargas, Berlin (DE)

Correspondence Address: MAYER & WILLIAMS PC, 251 NORTH AVENUE WEST, 2ND FLOOR, WESTFIELD, NJ, 07090, US

(73) Assignee: Forschungsverbund Berlin e. V., Berlin (DE)

(21) Appl. No.: 11/920,335

(22) PCT Filed: Apr. 28, 2006
(86) PCT No.: PCT/DE2006/000779

§ 371 Date: Mar. 5, 2009

(30) Foreign Application Priority Data
May 9, 2005 (DE) 10 2005 022 182.3

(51) Int. Cl. G06F 19/00; C07D 277/04; C07C 61/00; C07D 263/58

(52) U.S. Cl. 702/19; 548/146; 562/509; 548/221

(57) ABSTRACT

The invention relates to compounds which bind to the PDZ domains of proteins with PDZ domains, the uses of such compounds and screening methods for identification of such compounds.

### RELATED APPLICATIONS

This is a national stage application based on PCT/DE2006/000779, filed Apr. 28, 2006, which claims priority from DE 10 2005 022 182.3, filed May 9, 2005.

### FIELD OF THE INVENTION

The invention relates to new substances, which bind to the PDZ-domain of proteins, the uses of substances, which bind to the PDZ-domain of proteins, and means for identification of compounds, which bind to the PDZ-domain of a protein.

### PRIOR ART AND BACKGROUND OF THE INVENTION

Originally, the PDZ-domain was identified as a common element in three structurally related proteins, namely PSD-95/SAP90, DLG and ZO-1 (Garner et al., TRENDS IN CELL BIOL., 6:429-433 (1996); Craven et al., CELL, 93:495-498 (1998); Hutter et al., NEURO SCI. Res., 32:1-7 (1998)). The PDZ-domain is also called DLG homology region (DHR) or GLGF Repeat. The latter is based on the presence of a Gly-Leu-Gly-Phe sequence motive. The PDZ-domain comprises approx. 90 amino acids, and crystallographic investigations of PSD-95, SAP97 and CASK show that it is formed of two alpha helices and six beta sheets (Daniels et al., NAT. STRUCT. BIOL. 5:317-325 (1998); Doyle et al., CELL, 85:1067-1076 (1996)).

PDZ-domains, which can be found 785 times in 436 different human genes, belong to one of the most important protein classes in the human genome (Kay et al., Chemistry & Biology, 11:423-424 (2004)). PDZ-domains control the localization, the clustering, the recycling and the cell membrane expression of many receptor, transport and ion channel proteins (Dev, K.K., Nat. Rev. Drug Discov. 3:1047-1056 (2004)).

By recruiting downstream proteins in signalization pathways, PDZ-domains mediate the generation of specific multi-protein complexes. Proteins, which contain PDZ-domains, play an important role in many key pathways, including conservation of the polarity and morphology of epithelial cells, organization of the postsynaptic density in neural cells, and regulation of the activity and transport of membrane proteins. The consequence of this is that substances, which bind to the PDZ-domain, can specifically modulate such proteins or protein complexes and have therefore a particular therapeutic potential.

This principle of modulation of PDZ-domains could be shown by blocking peptide ligands in different cell culture and animal models (Dev, K.K., Nat. Rev. Drug Discov. 3:1047-1056 (2004)). Focal brain damages by ischemia in a rat model could be reduced by peptide ligands, which block the interaction between the PDZ-domain of the protein PSD-95 and N-methyl-D-aspartate receptors (NMDARs), which is a new approach for the therapy of strokes (Aarts et al., Science 298:846-850 (2002)). Furthermore, it could be shown that the blocking of the second PDZ-domain of the protein MAGI3 by irreversible, synthetic small-molecule inhibitors leads to a three times higher activity of a cancer-relevant enzyme in a cell culture model [Fujii et al., J. Am. Chem. Soc. 125:12074-12075 (2003)).

### TECHNICAL OBJECT OF THE INVENTION

It is therefore the technical object of the invention to indicate compounds, which are capable to bind to the PDZ-domain of a protein, where in absence of a modulator the interaction with natural protein ligands takes place. Further, it is the technical object of the invention to indicate means for identification of such compounds.

### BRIEF DESCRIPTION OF THE DRAWINGS

FIG. 1A-1B, shows compounds 1-8 according to the inventions 1-2, 4-7, 16-21, which bind to the PDZ-domain of the protein AF6.

FIGS. 2A-2B, shows compounds 9-21 according to the inventions 1-2, 4-7, 16 and 21, which bind to the PDZ-domain of the protein AF6.

FIGS. 3A-3D, shows compounds 22-40 that do not bind to the PDZ-domain of the protein AF6.

FIG. 4 shows usable compounds according to the invention that bind to the PDZ-domain.

FIG. 5 shows usable compounds according to the invention that bind to the PDZ-domain.

FIGS. 6A, 6B and 6C show the structural models for the complex of compound 1 and the PDZ-domain of AF6. The protein backbone assembly of the 20 lowest-energy structural models in FIG. 6A (stereo view) shows a low standard deviation of the coordinates in the secondary structure elements.

FIG. 7A shows the superimposition of ¹H-¹⁵N HSQC spectra of the PDZ-domain of AF6 comparing proteins without ligand and with 0.75 μM of compound 1.

FIG. 7B shows the superimposition of ¹H-¹⁵N HSQC spectra of the PDZ-domain of Syntrophin comparing protein Syntrophin without ligand and with 0.200 μM of compound 1.

FIG. 8A-8B is a systematic selection of test compounds, which changes of the binding affinity and of the binding site occupation are experimentally obtained when employing the method. Each column in FIG. 8 shows the amino acid residues of the PDZ-domain of AF6, the backbone amide resonances for addition of the specified compound in 8 to 10-fold ligand excess in the previously described NMR assay show chemical shifts. Herein, amino acid residues are abbreviated by the internationally used 1-letter code. The numbering of the amino acid residues is identical with the numbering in the protein-ligand complex in Table 1. The chemical shifts are split up into the 2 categories “large (bold italics)” and “small (normal letters)”

FIG. 9 shows a binding curve for the binding of compound 1 to the PDZ-domain of AF6.

### BASICS OF THE INVENTION AND PREFERRED EMBODIMENTS

For achieving this technical object, the invention teaches the use of a compound according to Formula I

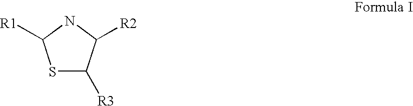

wherein R1 and R2 are ═O or ═S and are identical or different, wherein R2 may alternatively be two —H, wherein R3 is ═CHR4, —CH₂R4 or —CHR5R4, wherein R4 is phenyl; phenyl simply, doubly or triply substituted with -Hal or —C(Hal)ₙ (n=1, 2, or 3); 2-, 3-, 4-, or 5-thienyl; 2-, 3-, 4-, or 5-thienyl simply, doubly or triply substituted with -Hal or —C(Hal)ₙ (n=1, 2, or 3): 2-, 3-, 4-, or 5-furyl; 2-, 3-, 4-, or 5-furyl simply, doubly or triply substituted with -Hal or —C(Hal)ₙ (n=1, 2, or 3); or C1-C5 alkyl, linear or branched, wherein -Hal is —F, —Cl, —Br, or —I, wherein R5 is C1-C5 alkyl, linear or branched, or —CH₂—CO—N(R6)₂ with R6=C1-C5 alkyl, linear or branched, wherein free valences of the ring are bound with hydrogen, wherein the ring —S— may be replaced by —O—, —CH₂— or —CO—, or a physiologically well-tolerated salt of such a compound for preparing a pharmaceutical composition for modulation of a protein containing a PDZ-domain.

Generally, the term C1 to C5 includes alkyl methyl, ethyl, propyl, isopropyl, butyl, isobutyl, tert-butyl, pentyl, isopentyl, tert-pentyl and neopentyl.

Preferred substances are characterized by that R1 is ═S, wherein R2 is ═O, wherein R3 is ═CH—R4, and/or that R4 is 3-thienyl, phenyl, or phenyl substituted in para with -Hal, preferably —Br, and/or that R1 is ═S, wherein R2 is ═O, wherein R3 is —CH₂R4 or —CHR5R4, and R4 is 3-furyl or phenyl substituted in para with —CHal₃, preferably —CF₃, wherein R5 is methyl or —CH₂—CO—N(CH₃)₂, and/or that R1 is ═O, wherein R2 is ═O, wherein R3 is ═CH—R4, and/or R4 is isopropyl or phenyl substituted in meta or para with —CHal₃, preferably —CF₃, and/or R1 is ═O, wherein R2 is ═O, wherein R3 is —CH₂—R4, and wherein R4 is phenyl substituted in para with -Hal, in particular —Br, or with —CHal₃, in particular —CF₃.

Furthermore, the invention teaches the use of a compound according to Formula II

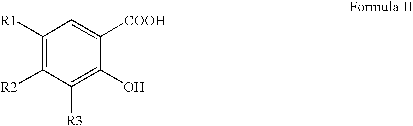

wherein R1 is -Hal or C1-C5 alkyl, linear or branched, wherein R2 is —H, and wherein R3 is —NO₂, -Hal, C1-C5 alkyl, linear or branched, wherein instead of R1 and R2 a ring with —CH═CHal-CH═CH— may be formed, or wherein instead of R2 and R3 a ring with —CH═CH—CH═CH— may be formed, wherein -Hal is —F, —Cl, —Br, or -J, wherein free valences of the ring are bound with hydrogen, or a physiologically well-tolerated salt of such a compound for preparing a pharmaceutical composition for modulation of a protein containing a PDZ-domain.

Then it is preferred that R3 is —Br or tertiary-butyl, and/or R1 is —Cl, —Br, or tertiary-butyl, and/or that R1 is —Br, wherein instead of R2 and R3 a ring with —CH═CH—CH═CH— is formed, and/or that R3 is —Br, wherein instead of R1 and R2 a ring with —CH═CBr—CH═CH— is formed.

Furthermore, the invention teaches the use of a compound according to Formula III

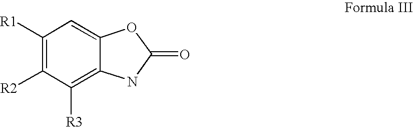

wherein R1 is —NO₂ or -Hal, wherein R2 and R3 are identical or different and are —H or -Hal, wherein -Hal is —F, —Cl, —Br, or -J, wherein free valences of the rings are bound with hydrogen, or a physiologically well-tolerated salt of such a compound for preparing a pharmaceutical composition for modulation of a protein containing a PDZ-domain.

Then it is preferred that R1, R2 and R3 are -Hal, in particular —Cl, and/or that R1 is —NO₂, wherein R2 and R3 are —H.

A pharmaceutical composition according to the invention is prepared by mixing the compound in a physiologically effective dose with at least one auxiliary or carrier substance.

This galenic preparation of a pharmaceutical composition according to the invention may be performed in a conventional manner. As counter ions for ionic compounds may for instance be used Na+, K+, Li+ or cyclohexylammonium. Suitable solid or liquid galenic forms of preparation are for instance granulates, powders, dragees, tablets, (micro) capsules, suppositories, syrups, juices, suspensions, emulsions, drops or injectable solutions (i.v., i.p., i.m., s.c.) or fine dispersions (aerosols), transdermal systems, and preparations with protracted release of active substance, for the production of which usual means are used, such as carrier substances, explosives, binding, coating, swelling, sliding or lubricating agents, tasting agents, sweeteners and solution mediators. As auxiliary substances are named here magnesium carbonate, titanium dioxide, lactose, mannite and other sugars, talcum, milk protein, gelatin, starch, cellulose and derivatives, animal and vegetable oils such as cod-liver oil, sunflower, peanut or sesame oil, polyethylene glycols and solvents, such as sterile water and mono or multi-valent alcohols, for instance glycerin.

As indications are mentioned here: cancer, schizophrenia, depressions and states of anxiety, Parkinson's disease, Huntington's disease, Alzheimer's disease, epilepsy, chronic and neuropathic pain, aberrations of the hormone-regulated food intake. PDZ-domains play an important role for the modulation of proteins, which are associated with these diseases (Dev, K.K., Nat. Rev. Drug Discov. 3:1047-1056 (2004)).

A plurality of the compounds mentioned above and falling under Formula I are not known from prior art. Therefore, the invention also teaches a compound according to Formula I

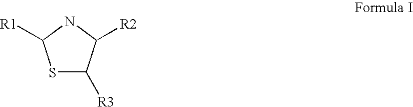

wherein R1 and R2 are ═O or ═S and are identical or different, wherein R2 may alternatively be two —H, wherein R3 is ═CHR4, —CH₂R4 or —CHR5R4, wherein R4 is phenyl; phenyl simply, doubly or triply substituted with -Hal or —C(Hal)ₙ (n=1, 2, or 3); 2-, 3-, 4-, or 5-thienyl; 2-, 3-, 4-, or 5-thienyl simply, doubly or triply substituted with -Hal or —C(Hal)ₙ (n=1, 2, or 3); 2-, 3-, 4-, or 5-furyl; 2-, 3-, 4-, or 5-furyl simply, doubly or triply substituted with -Hal or —C(Hal)ₙ (n=1, 2, or 3); or C1-C8 alkyl, linear or branched, wherein -Hal is —F, —Cl, —Br, or -J, wherein R5 is C1-C5 alkyl, linear or branched, or —CH₂—CO—N(R6)₂ with R6=C1-C5 alkyl, linear or branched, wherein free valences of the ring are bound with hydrogen, wherein R4 is not 2-furyl or 2-thienyl, if R1 is ═O or ═S, and if R3 is ═CH—R4, wherein R4 is not phenyl or phenyl substituted in meta or para with —CF₃ or —Br, if R1 is ═O or ═S, if R2 is ═O, and if R3 is ═CH—R4 or —CH₂—R4, wherein R4 is not phenyl, if R1 is ═O or ═S, if R2 is ═O, and if R3 is ═CH—R4, wherein R4 is not phenyl substituted in para with —Br, if R1 and R2 are ═O, and if R3 is —CH₂—R4, wherein R4 is not isopropyl, if R1 is ═S, if R2 is ═O, and if R3 is ═CH—R4, wherein the ring —S— may be replaced by —O—, —CH₂— or —CO—, or a physiologically well-tolerated salt of such a compound.

Herein it is preferred that R1 is ═S, wherein R2 is ═O, wherein R3 is ═CH—R4, and/or that R4 is 3-thienyl, phenyl, or phenyl substituted in para with -Hal, preferably —Br, and/or that R1 is ═S, wherein R2 is ═O, wherein R3 is —CH₂—R4, and R4 is 3-furyl or phenyl substituted in para with —CHal₃, preferably —CF₃, and/or that R1 is ═O, wherein R2 is ═O, wherein R3 is ═CH—R4, and/or that R4 is isopropyl or phenyl substituted in meta or para with —CHal₃, preferably —CF₃, and/or that R1 is ═O, wherein R2 is ═O, wherein R3 is —CH₂—R4, and wherein R4 is phenyl substituted in para with -Hal, in particular —Br, or with —CHal₃, in particular —CF₃, and wherein R5 is methyl or —CH₂—CO—N(CH₃)₂, and/or that R1 is ═S, wherein R2 is ═O, wherein R3 is —CHR5R4, and wherein R4 is phenyl substituted in para with -Hal, in particular —Br, or with —CHal₃, in particular —CF₃, and wherein R5 is methyl or —CH₂—CO—N(CH₃)₂.

The above compounds bind to the PDZ-domain of proteins and are thus suitable to modulate the respective proteins or the complexes formed by these proteins in the cell, i.e. to activate or inhibit them. Thus, it would for instance be recommendable to inhibit such proteins, which turned out to be differentially expressed at the event of a disease, for instance a cancer disease, i.e. regulated up or down in correlation with the disease.

Subject matter of the invention is further a method for identification of a modulator of a protein containing a PDZ-domain, wherein a structural model of a modulator candidate optionally is first compared to a structural model of a reference compound, which binds to the PDZ-domain, and is pre-selected in case of an overlap of bioisosteric atoms, and wherein the structural model of the if applicable pre-selected modulator candidate is compared with a structural model of the protein or a structural model of a complex of protein and modulator candidate is investigated and it is determined, whether the modulator candidate binds to the PDZ-domain, wherein the structural model of the protein or of the complex is derived i) from structural coordinates of the complex with the reference compound, ii) from a fragment of the complex containing a PDZ-domain with the reference compound, or of a homolog to i) or ii). Basically, this is a screening method based on a structural model, wherein prospective compounds, in particular compounds with a molecular weight under 5,000 down to 1,000 or less, are so to speak “tried on” with the structural model of the PDZ-domain or of the protein. If by the comparison a binding is detected, the prospective compound is selected and may then be submitted to further tests for the development of pharmaceutical compositions.

A preferred variant of the method according to the invention is that the structural model of the protein or of the complex is obtained with a reference compound, which is compound 1 (Formula I, with R1=═S, R2=═O, R3=—CH₂—R4 and R4=phenyl substituted in para with CF₃). Herein, the protein in the complex assumes a particular conformation, which does not occur in any of the previously published structural models of PDZ-domains and comprises a new hydrophobic binding pocket, which can offer a decisive contribution to the binding strength of the modulator candidate. Therefore, the structural coordinates according to the invention of the protein in the complex with compound 1 present the protein in a particular small-molecule binding conformation according to the invention, which is advantageous for the success of the method according to the invention.

The comparison consequently can take the structural model of the complex of the protein with compound 1 as a model for matching new, unknown test compounds with the bound compound 1. This method is called ligand-based virtual screening. If herein bioisosteric atoms of a modulator candidate overlap with the atoms of compound 1, without repulsive overlappings with the protein taking place, the modulator candidate can be identified as a new modulator. If a modulator candidate in addition to the above conditions fills up one or several further binding pockets of the protein, which are not covered by compound 1, the modulator candidate can be identified as a new modulator with a higher affinity and/or a higher specificity.

The comparison can consequently comprise the combination of the structural model of the modulator candidate, and that after a pre-comparison of the modulator candidate with compound 1, with the structural model of the protein, and that based on the structural coordinates according to the invention of the complex, wherein optionally the free binding energy of the binding between modulator candidate and protein is determined, and wherein with low free binding energy a high binding probability is detected. The free binding energy can be calculated: a) by addition of the free energies of interatomic contacts between the structural model of the modulator candidate and the structural model of the protein, and/or b) by determination of the free binding energy between the force field of the modulator candidate and the force field of the protein.

The invention consequently also relates to the use of the structural coordinates in the small-molecule binding conformation with compound 1 i) of the protein AF6, ii) of a fragment of AF6 containing a PDZ-domain, or iii) of a homolog to i) or ii) for identification of a modulator of a protein containing a PDZ-domain, preferably in a screening method according to the invention. The invention further comprises a machine-readable storage medium containing machine-readable data, which after reading-out and processing by means of a data processing system with a suitable software provide a representation of the structural model according to the invention of a protein or of a complex, a computer software with a software code for carrying-out a method according to the invention or a use according to the invention, and a data processing system comprising a computer software according to the invention and a machine-readable storage medium according to the invention.

In the following, the invention is explained in more detail with reference to embodiments.

### Example 1

### Substances According to the Invention

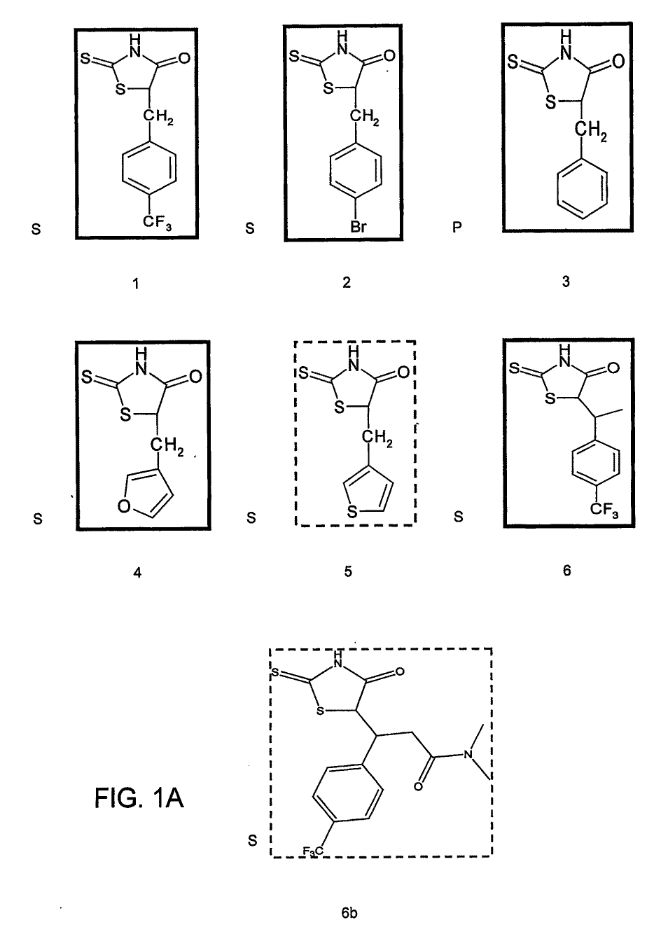

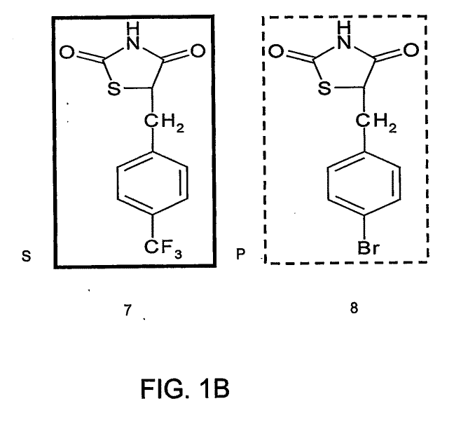

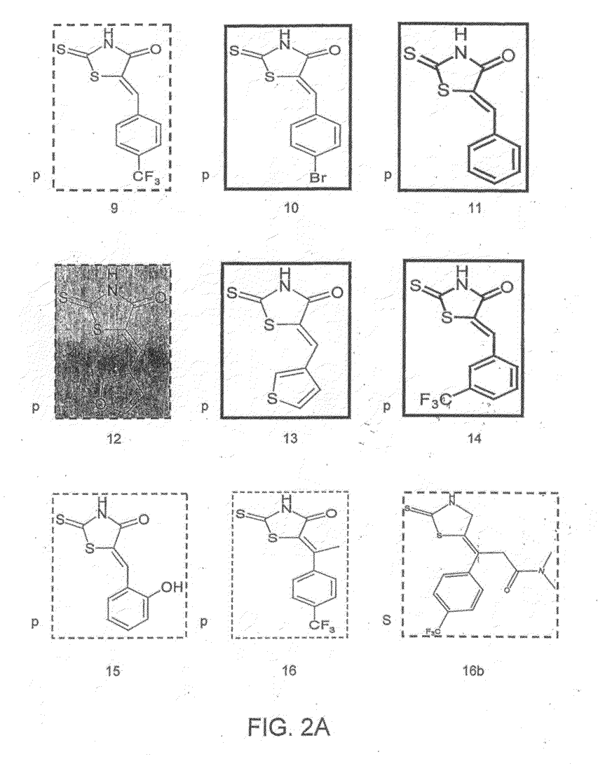

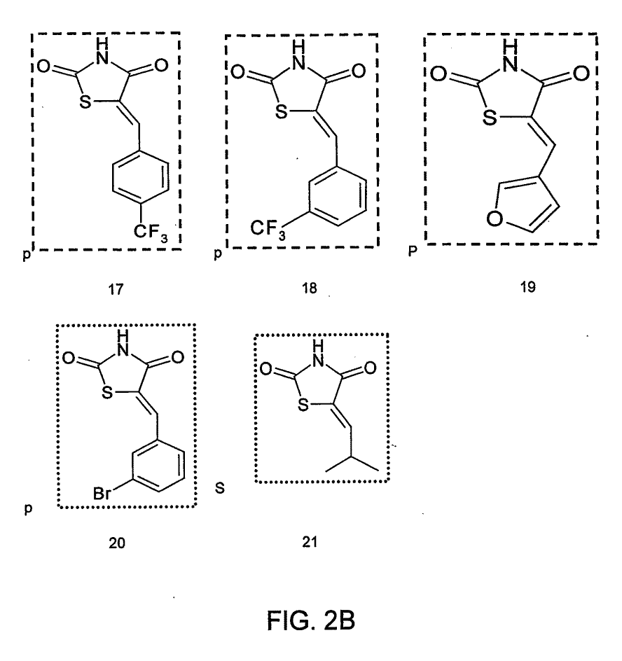

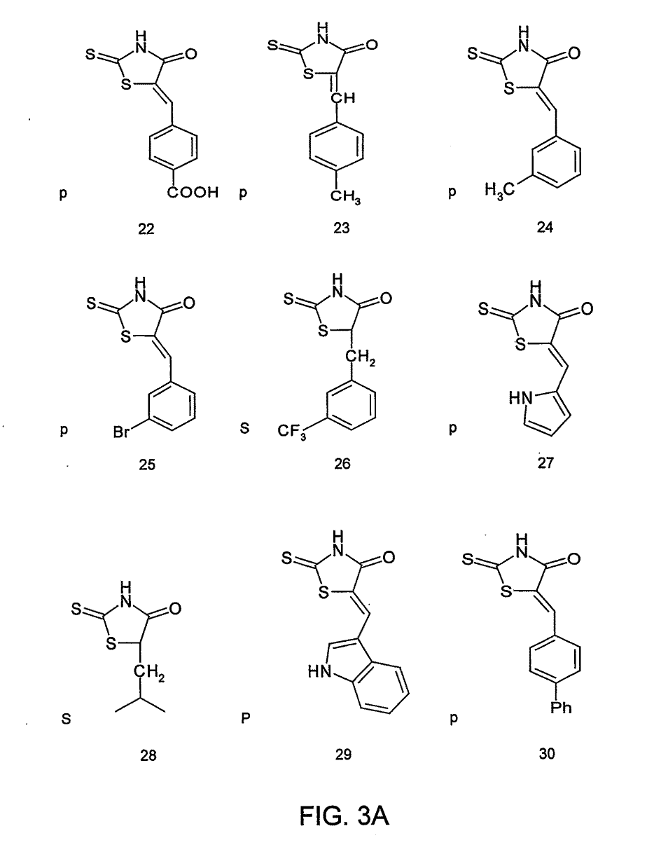

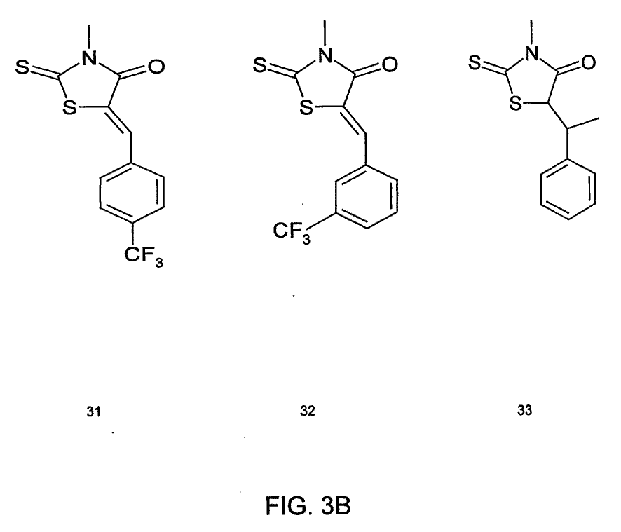

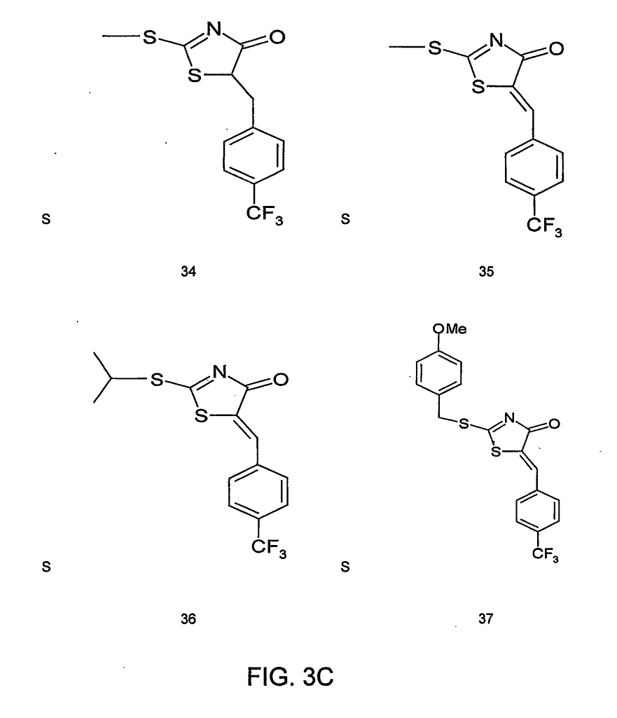

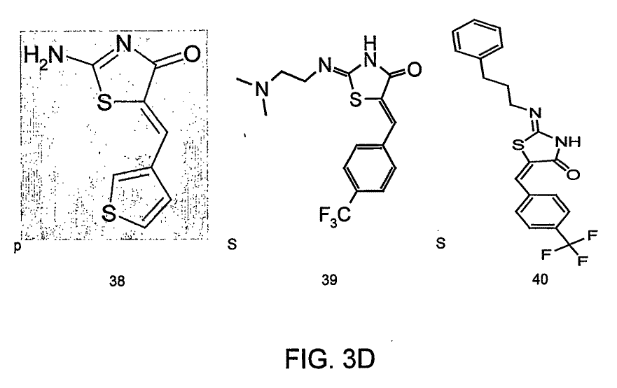

In FIGS. 1A, 1B, 2A, 2B, the new compounds according to the invention 1-2, 4-7, 16 and 21 are shown, which bind to the PDZ-domain of the protein AF6. In FIGS. 1A, 1B, 2A, 2B, the prior art compounds 3, 8, 9-15 and 17-20 are shown, which are suitable for the uses according to the invention, since they also bind to the PDZ-domain of the protein AF6. In FIGS. 3A to 3D, however, compounds are shown, which are no compounds according to the invention, since they do not bind to the PDZ-domain of the protein AF6.

An “S” marks new substances, which were synthesized. “p”, “P” and “R” are prior art substances, wherein commercially available substances are marked with “p”. Substances marked with “P” have been published—with synthesis pathways. Strong-binding substances are in addition framed with a continuous line. Weak-binding substances are in addition framed with a broken line. Non-binding substances do not have an additional frame.

When the non-binding compounds 31 and 32 in FIG. 3B are compared with the binding compounds 9 and 14 in FIG. 2A, it can be seen that the ring nitrogen in the 4-oxo-2-thiazolidinethione ring must not carry a substituent except possibly hydrogen. When the compounds 1 and 2 in FIG. 1A are compared with the compounds 7 and 8 in FIG. 1B, it can be seen that the residue R1 may be doubly bound sulfur as well as oxygen. The comparison between the non-binding compound 38 in FIG. 3D and the binding compound 13 in FIG. 2A shows, however, that R1 must not be an amino group. The R1 substituents alkyl-imino (compounds 39 and 40 in FIG. 3D) or alkyl-thio (compounds 34-37 in FIG. 3C) lead to non-binding derivatives, too.

A comparison between the compounds in FIG. 1A and FIG. 2A shows that the residue R3 may be singly as well as doubly bound to the ring. Depending on the respective residue R3, the compounds with singly bound R3 show a stronger binding (e.g. compound 1) or in rare cases also a weaker binding (compound 26 in FIG. 3A) than the doubly bound equivalents.

Compounds with R3=—CH₂—R4 and R4 being isopropyl (compound 21), furanyl (compounds 4), thiophenyl (compound 5) and six-membered aromatic rings with small substituents of one or no C atom at all show a binding (compound 1, 3), whereas six-membered aromatic rings with larger substituents of more than two C atoms (compounds 29, 30) do not show any binding. Small substituents at the six-membered aromatic ring of R4 may in principle be at the ortho (compound 15), meta (compound 14) or also para (compound 1) positions, however the substitution position leading to a binding depends on the type of the substituent as well as on variations in other parts of the compound.

A particularly strong activity, i.e. binding to the PDZ-domain, show the following compounds: 1, 7, 9, 6 and 2.

### Example 2

### Other Usable Substances According to the Invention

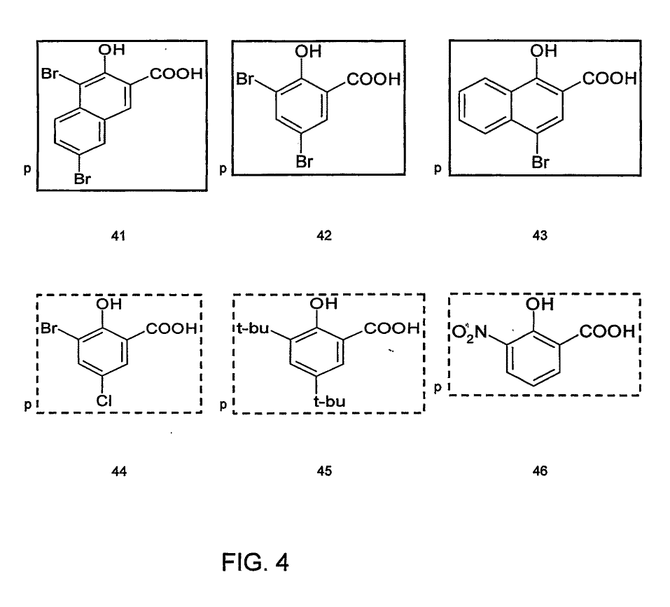

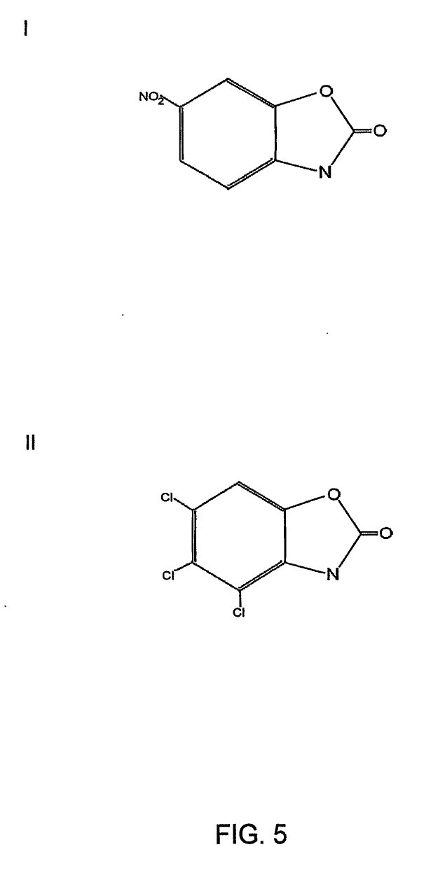

FIGS. 4 and 5 show other usable compounds according to the invention, which bind to the PDZ-domain. With regard to the markings, the explanations in Example 1 apply in a corresponding manner.

### Example 3

### Synthesis Examples

### Example 3.1

### Synthesis of Compounds 9-15 in FIG. 2A and Compounds 22-25, 27 and 29-30 in FIG. 3A

A mixture of 2,4-thioxothiazolidine-one (20 mmol), aryl or alkyl aldehyde (20 mmol), piperidine (16 mmol) and EtOH is cooked for 18-24 h under reflux, then poured into H₂O and acidified with acetic acid. The solid reaction product is recrystallized from methanol, methano/water or ethanol/water solvency.

### Example 3.2

### Synthesis of the Compounds of FIG. 2B

The compounds in FIG. 2B can be prepared according to Example 3.1 by using 2,4-thiazolidinedione instead of 2,4-thioxothiazolidine-one.

### Example 3.3

### Synthesis of Compounds 1-5 in FIG. 1A and Compounds 26 and 28 in FIG. 3A

These compounds can be prepared according to Example 3.1, followed by the following steps: A 2.0-molar solution of lithium borohydride (2.2 equivalents) in tetrahydrofuran (THF) is dropped under stirring into a solution of 5-arylidene-4-oxo-2-thiazolidinethione in pyridine and THF under nitrogen atmosphere at room temperature. The mixture is heated under reflux, until the reduction reaction is accomplished (approx. 3 to 5 h). The mixture is then cooled down, carefully added into a diluted hydrochloric acid solution in distilled water at 5° C. and multiply extracted with ethyl acetate. The ethyl acetate extracts are combined, washed with water, dried over MgSO₄ and concentrated at the rotation vaporizer. The raw product is purified by means of silica gel flash chromatography, wherefrom the final product results.

### Example 3.4

### Synthesis of the Compounds According to FIG. 1B

The compounds in FIG. 1B can be prepared according to Example 3.2 followed by the following steps: A 2.0-molar solution of lithium borohydride (2.2 equivalents) in tetrahydrofuran (THF) is dropped under stirring in a solution of 5-arylidene-2,4-thiazolidinedione in pyridine and THF under nitrogen atmosphere at room temperature. The mixture is heated under reflux, until the reduction reaction is accomplished (approx. 3 to 5 h). The mixture is then cooled down, carefully added into a diluted hydrochloric acid solution in distilled water at 5° C. and multiply extracted with ethyl acetate. The ethyl acetate extracts are purified, washed with water, dried over MgSO₄ and concentrated at the rotation vaporizer. The raw product is purified by means of silica gel flash chromatography, wherefrom the final product results.

### Example 3.5

### Synthesis of Compound 33 in FIG. 3B

Compound 33 in FIG. 3B can be prepared as follows: 60-% dispersion of sodium hydride (0.12 g, 3 mmol) in mineral oil is washed with hexane. The sodium hydride is suspended in THF and stirred for 10 min at 0° C. A solution of N-methylrhodanine (0.735 g, 3 mmol) in THF is added portion by portion under stirring into the suspension of sodium hydride in THF. Then (I-bromoethyl)benzene (0.55 g, 3 mmol) is added in one portion, stirred for 1 h and extracted with chloroform. The chloroform extracts are combined and the solvent is removed under vacuum. The raw product is purified by means of silica column chromatography in an ethyl acetate/hexane=1/9 mixture, in order to obtain the final product.

### Example 3.6

### Synthesis of Compounds 31 and 32 in FIG. 3B

Compounds 31 and 32 in FIG. 3B can be prepared as follows: To a mixture of N-methylrhodanine (0.735 g, 5 mmol) and aryl aldehyde (5 mmol) in a minimum volume of dichloromethane, potassium fluoride on aluminum (2 g) is added. The solvent is removed under vacuum and the remaining solids are irradiated at 150° C. for 10 min with microwaves. The reaction products are extracted with dichloromethane, filtered over Celite and concentrated up. The raw product is purified by means of silica gel flash chromatography, wherefrom the final product results.

### Example 3.7

### Synthesis of Compound 16 in FIG. 2A

Compound 16 in FIG. 2A can be prepared as follows: A mixture of 2,4-thioxothiazolidine-one (0.66 g, 5 mmol), 4′-(trifluoromethyl)-acetophenone (0.94, 5 mmol), piperidine (0.40 mL, 4 mmol) and THF is irradiated for 10 min at 100° C. with microwaves. The raw product is purified by means of silica gel flash chromatography, wherefrom the final product results.

### Example 3.8

### Synthesis of Compound 6 in FIG. 1A

Compound 6 in FIG. 1A can be prepared according to Example 3.7, followed by the further synthesis pathway according to Example 3.9.

### Example 3.9

### Synthesis of Compounds 35-37 in FIG. 3C

Compounds 35-37 in FIG. 3C can be prepared as follows: A mixture of 5-(4-trifluoromethylbenzylidene)-4-oxo-2-thiazolidinethione (10 mmol) in aqueous NaOH (2%, 25 mL) is stirred with methyl iodide (compound 35), isopropyl iodide (compound 36) or p-methoxybenzyl iodide (compound 37) (11 mmol each) at room temperature over night. The raw product is purified by means of silica gel flash chromatography, wherefrom the final product results.

### Example 3.10

### Synthesis of Compounds 39 and 40 in FIG. 3D

The compounds 39-40 in FIG. 3D can be prepared as follows: A mixture of 5-(4-trifluoromethylbenzylidene)-4-oxo-2-thiazolidinethione (1 mmol) and 2-dimethylamino-ethylamine or 3-phenyl-propylamine (1 mmol) in THF (1.5 mL) is irradiated for 15 min at 120° C. with microwaves. The raw product is purified by means of silica gel flash chromatography, wherefrom the final product results.

### Example 4

### 3D Structure

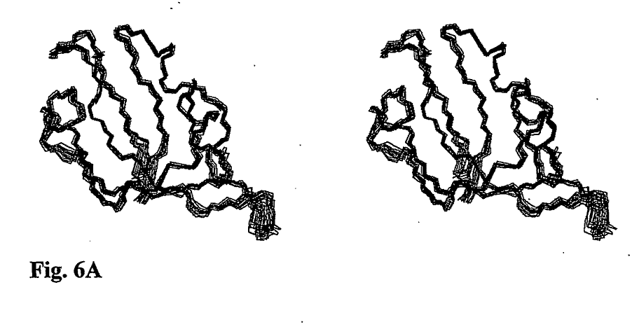

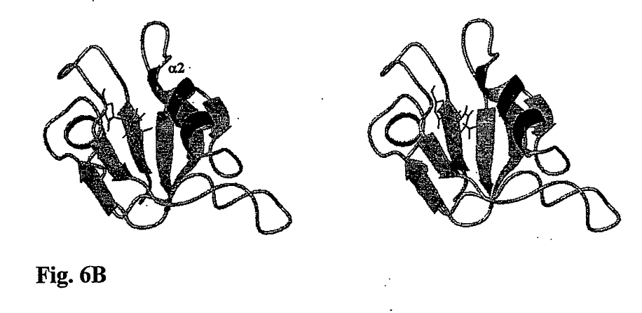

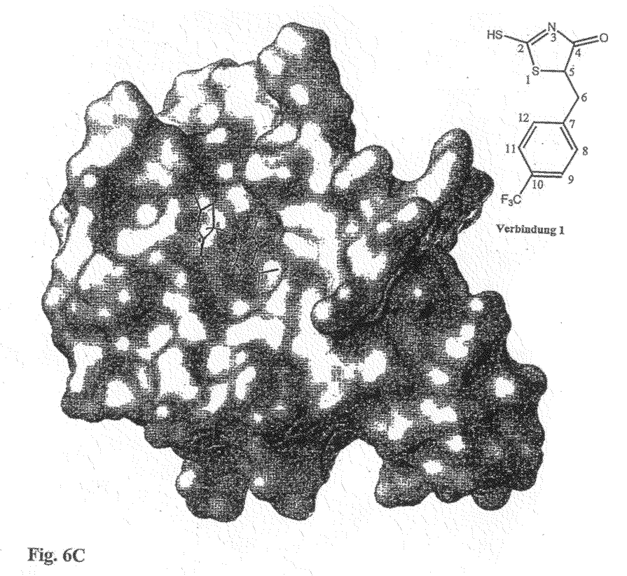

A complex of the human protein AF6 with compound 1 of FIG. 1A was investigated by means of NMR spectroscopy with regard to its 3D structure. In detail, this was made as follows: The 15N and 15N/13C isotope-marked protein of the PDZ-domain of AF6 was prepared as described above [Boisguerin, P. et al., Chem. Biol. 11:449-459 (2004)]. For the NMR protein backbone assignment, a sample of 1.3 mM 15N/13C marked PDZ-domain in 20 mM phosphate buffer (pH 7.0), 50 mM NaCl, complete [R] protease inhibitor and 10% (v/v) D2O was used. NMR spectra were recorded at 300 K on a Bruker DRX600 spectrometer with an inverted triple-resonance sample head with Z gradient. The NMR raw data were processed with the software XWIN-NMR and analyzed with the software Sparky. For the NMR protein backbone assignment, the triple-resonance NMR experiments CBCA(CO)NNH/CBCANNH and HA(CO)NNH/HANNH and some side chains-selective experiments as described in [Wiedemann, U. et al., J Mol Biol., 343(3):703-18 (2004)] were recorded. For the side chains NMR assignment, the following NMR experiments were recorded and evaluated: 3D HBHA(CO)NH, H(CCCO)NH-TOCSY, (H)CC(CO)NH-TOCSY with the sample for the backbone assignment, and 3D HCCH—COSY, HCCH-TOCSY and 3D-13C-NOESY-HMQC with the same sample after transfer in 100% (v/v) D2O. The NMR experiments were performed as described [Kay, L. E. et al., Biochem Cell Biol., 75(1):1-15 (1997)]. Interproton distances for the 3D structure determination were obtained by recording and evaluating the following NMR experiments: 3D-15N-NOESY-HSQC (80 ms) and 3D-13C-NOESY-HMQC (80 ms). 3D structural models were calculated by means of the software Cyana [Guntert P., Mol Biol., 278:353-78 (2004)]. The 20 lowest-energy 3D structural models (FIG. 6A) were analyzed and tested by means of the software MOLMOL [Koradi R., et al., J Mol Graph., 14(1):51-5, 29-32 (1996)] and the software PROCHECK-NMR [Laskowski R. A., et al., J Biomol NMR, 8(4):477-86 (1996)]. The protein coordinates of the lowest-energy structural model from the calculation with the software Cyana and the protein ligand-interproton distances from the NMR experiments were used as input data for a computer-based docking of the ligand-protein complex. For this purpose, a 2 nanosecond molecular dynamics simulation of the input data was performed in a octahedrally limited water box by means of the software AMBER 8.0. The finally obtained coordinates of the protein-ligand complex are shown in Table 1.

FIGS. 6A, 6B and 6C show the obtained structural models for the complex of compound 1 and the PDZ-domain of AF6. The protein backbone assembly of the 20 lowest-energy structural models in FIG. 6A (stereo view) shows a low standard deviation of the coordinates in the secondary structure elements, i.e. a good quality of the structural models in these regions being important for the ligand binding.

The protein backbone band model of the average structure of the 20 lowest-energy structures in the complex with compound 1 (FIG. 6B) is shown in the same orientation as in FIG. 6A. The 6 beta folding sheets and 2 alpha helices being characteristic for PDZ-domains can be seen. The secondary structure elements folding sheet β2 and helix α2 being important for the binding of natural peptide ligands are marked.

The surface representation of the PDZ-domain in the complex with compound 1 (FIG. 6C), which is also shown in the same orientation as in FIGS. 6A and 6B, shows, how in particular the trifluoromethyl-phenyl residue of compound 1 fits in a binding pocket of the protein.

Many atoms of this residue are nearly or completely in van-der-Waals distance to atoms of the protein, with the consequence that at these positions no substituents with more than two C atoms are allowed. This result is in accordance with the non-binding compounds 29 and 30 in FIG. 3A. By inspection of the structural model in FIG. 6C by means of the atom coordinates in Table 1 with the aid of a suitable software, it can be seen that at the atom positions 1, 2, 6, 11 and 12 even further substituents, which may in part be larger than a C atom or other heteroatoms, can be added, in order to generate improved compounds in the meaning of the invention. In contrast thereto, the N atom in position 3 is in such a close contact with the protein that here no substituents of the size of a C atom or larger can have space. This prediction is confirmed by the non-binding compounds 31-33 in FIG. 3B. In this sense, the use of the shown complex structure represents a method for finding new or/and improved ligands of PDZ-domains.

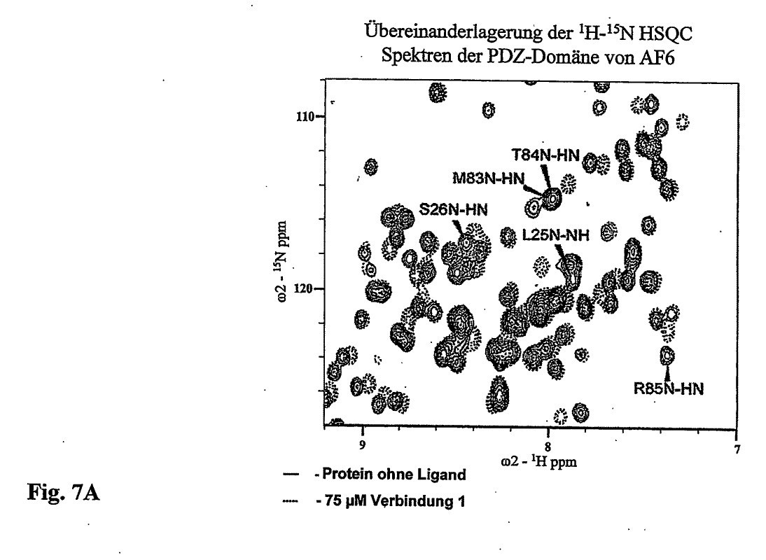

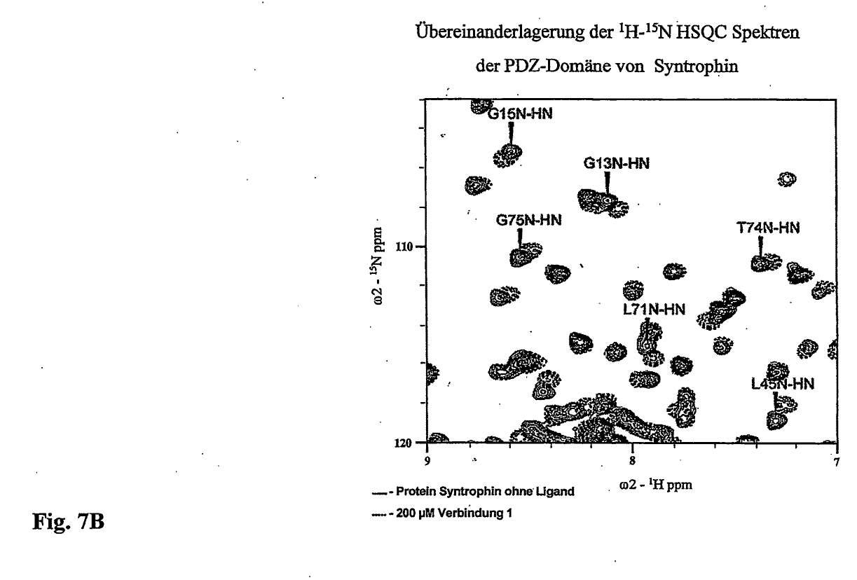

The ubiquitarily 15N marked PDZ-domains of the proteins AF6 [Boisguerin, P. et al., Chem. Biol. 11:449-459 (2004)] and Syntrophin [Schultz, J. et al., Nat Struct Biol., 5(1):19-24 (1998)] were prepared as described in the publications. For the NMR assay 50 μM 15N marked protein are mixed in a buffer of 20 mM Na phosphate pH 6.5, 50 mM NaCl and 10% d6-DMSO with a 8 to 10-fold molar excess of test ligand and a 15N-HSQC NMR spectrum is recorded at a temperature of 300 K on a Bruker DRX 600 MHz NMR spectrometer. Secondly, an identical comparison spectrum of protein in absence of test ligand is recorded. The NMR spectra are processed with the aid of the software XWIN-NMR and compared with each other with the aid of the software Sparky. When the comparison shows chemical shifts of more than one resonance and less than about half of all resonances, then it is a binding ligand. Exemplarily is shown such a comparison of spectra for the PDZ-domain of AF6 in FIG. 7A and for the PDZ-domain of Syntrophin in FIG. 7B.

For the determination of equilibrium dissociation constants (in short: Kd values), a test ligand is added step-wise in increasing concentrations to 50 μM protein solution, and for each concentration step a 15N-HSQC NMR spectrum is recorded under the same conditions as described above. The average chemical shift of a resonance, d, is calculated according to the following equation from the NMR raw data:

d=((ΔH)²+(ΔN/5)²)^{0.5}

wherein ΔH is the shift in ppm units along the 1H resonance axis, and ΔN is the shift in ppm units along the 15N resonance axis. The average shift, d, is drawn as a function of the concentration of the test ligand, and a binding curve results, to which is adapted the following mathematical equation:

y(Lo)=a−(a²−Lo/Po)^{0.5}

with

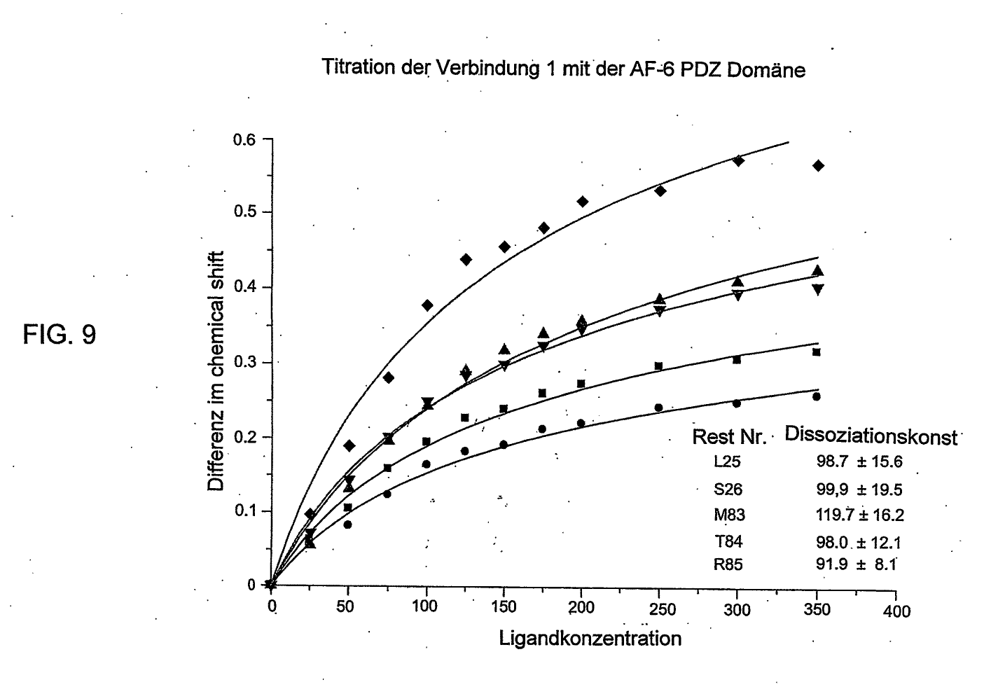

a=(Po+Lo+Kd)/(2*Po)

wherein Lo is the total concentration of ligands, Po is the total concentration of protein and Kd is the Kd value. This equation describes the law of mass action of a reaction “protein+ligand=complex”. The binding curve for the binding of compound 1 to the PDZ-domain of AF6 is shown in FIG. 9.

With the aid of a software such as e.g. “Origin 5.0”, the adaptation is optimized in a non-linear manner, thereby providing for each investigated resonance a microscopic Kd value. From the mean value of all relevant microscopic Kd values, a macroscopic Kd value is calculated. In Table 2, the macroscopic Kd values including the statistical standard deviation for the binding of some test ligands to the PDZ-domains of AF6 and of Syntrophin are shown.

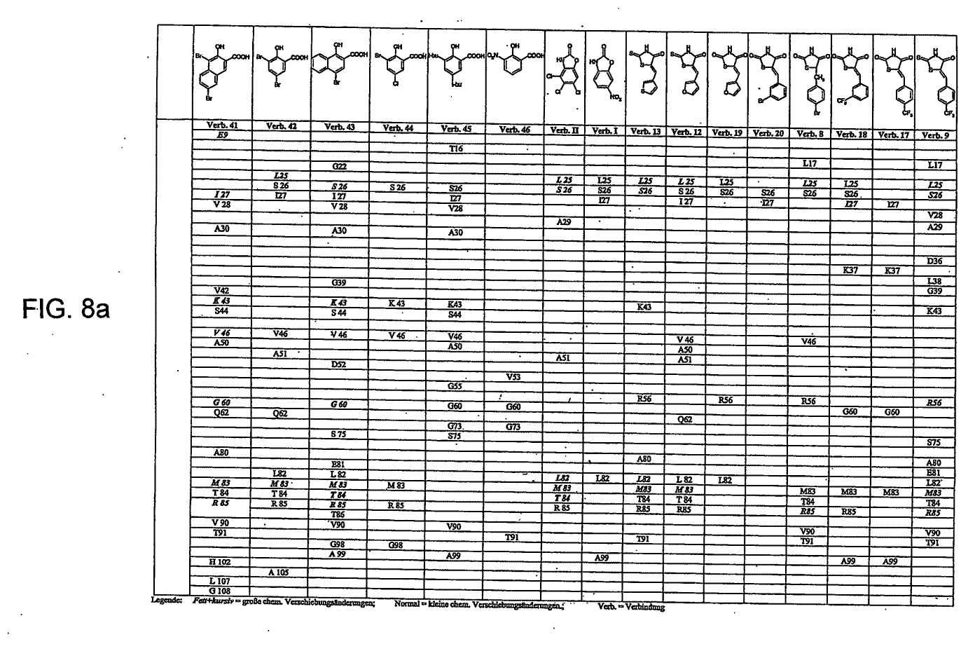

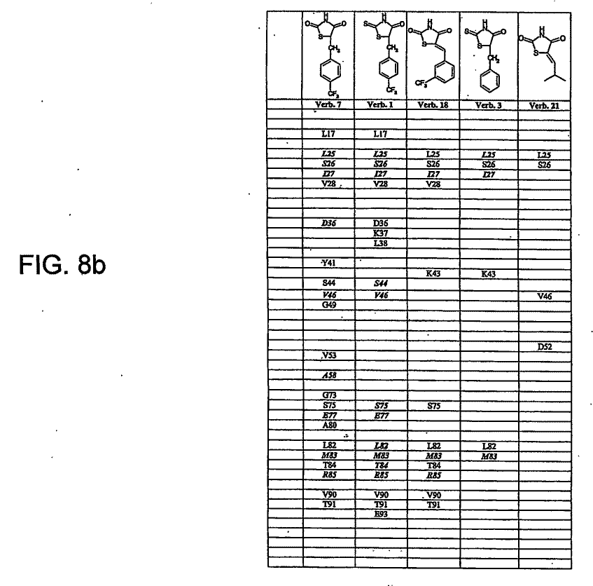

The basis for the complex structure-based method for finding new modulators is a correlation between chemical variations of compound 1, the binding affinity and the binding site occupation of the protein. FIG. 8 shows for a systematic selection of test compounds, which changes of the binding affinity and of the binding site occupation are experimentally obtained when employing the method. Each column in FIG. 8 shows the amino acid residues of the PDZ-domain of AF6, the backbone amide resonances for addition of the specified compound in 8 to 10-fold ligand excess in the previously described NMR assay show chemical shifts. Herein, amino acid residues are abbreviated by the internationally used 1-letter code. The numbering of the amino acid residues is identical with the numbering in the protein-ligand complex in Table 1. The chemical shifts are split up into the 2 categories “large (bold italics)” and “small (normal letters)”. From the shown amino acid residues, the binding site of a compound on the protein can be determined by color-highlighting the backbone amide atoms of this amino acid residues in the 3D protein structure of Table 1 and creating the largest common cut set of all these atoms on the protein surface.

Furthermore, from a comparison of the shown amino acid residues between two compounds, information about the differential occupation of partial binding sites can be obtained. When e.g. compound 1 is compared with compound 21, the amino acid residues L25, S26 and V46 occur in both compounds, however the residues S75 to T91 are missing in compound 21. Considering the position of these residues S75 to T91 in the 3D structural model of the protein compound complex of Table 1, a localization is found in the environment of the trifluoromethylphenyl residue of compound 1. Since this trifluoromethylphenyl residue in compound 21 is reduced to a substantially smaller isopropyl residue, the missing of the amino acid residues S75 to T91 in compound 21 can be correlated with a missing occupation of the partial binding site of the trifluoromethylphenyl residue. In an analogous manner, it follows from a comparison of the compounds 46 and 1 that compound 46 does not or only insufficiently occupy the binding sites L25 to V28 and S75 to T91 of compound 1.

For a selection of 3 compounds, the Kd values for the PDZ-domains of AF6 and Syntrophin are shown in Table 2. Considering the fact that the two PDZ-domains are proteins with only a low small-molecule binding capacity (low drugability), the Kd value of 101 μM for compound 1 (enantiomers mixture with regard to atom position 5) indicates an unexpectedly strong binding. In comparison therewith, a natural hexa-peptide ligand shows a Kd value of only 137 μM [Wiedemann, U. et al., J. Mol. Biol. 343(3):703-18 (2004)], although the hexa-peptide has a 2.3 times higher molecular weight than compound 1.

TABLE 1
Structural coordinates of the complex AF6 with compound 1

| | |
|---|---|
| HEADER | Molecular Docking by AMBER (2ns) |
| HEADER | Created by MANGESH JOSHI 27-APR-05 |
| COMPND | UNNAMED |
| REMARK | GENERATED BY SYBYL (TRIPOS, INC.) 27-APR-05 |

| | | | | | | | | | | | | | | |
|---|---|---|---|---|---|---|---|---|---|---|---|---|---|---|
| SEQRES | 1 85 | GLU | ILE | ILE | THR | VAL | THR | LEU | LYS | LYS | GLN | ASN | GLY | MET |
| SEQRES | 2 85 | GLY | LEU | SER | ILE | VAL | ALA | ALA | LYS | GLY | ALA | GLY | GLN | ASP |
| SEQRES | 3 85 | LYS | LEU | GLY | ILE | TYR | VAL | LYS | SER | VAL | VAL | LYS | GLY | GLY |
| SEQRES | 4 85 | ALA | ALA | ASP | VAL | ASP | GLY | ARG | LEU | ALA | ALA | GLY | ASP | GLN |
| SEQRES | 5 85 | LEU | LEU | SER | VAL | ASP | GLY | ARG | SER | LEU | VAL | GLY | LEU | SER |
| SEQRES | 6 85 | GLN | GLU | ARG | ALA | ALA | GLU | LEU | MET | THR | ARG | THR | SER | SER |
| SEQRES | 7 85 | VAL | VAL | THR | LEU | GLU | VAL | ALA | | | | | | |
| SEQRES | 1 1 | CPX | | | | | | | | | | | | |

| | | | | | | | | | |
|---|---|---|---|---|---|---|---|---|---|
| ATOM | 1 | N | GLU | 1 | −0.982 | −12.028 | −7.811 | 1.00 | 1.00 |
| ATOM | 2 | CA | GLU | 1 | −1.679 | −11.147 | −8.810 | 1.00 | 0.00 |
| ATOM | 3 | C | GLU | 1 | −2.017 | −9.812 | −8.256 | 1.00 | 0.00 |
| ATOM | 4 | O | GLU | 1 | −2.327 | −9.729 | −7.074 | 1.00 | 0.00 |
| ATOM | 5 | CB | GLU | 1 | −2.928 | −11.922 | −9.407 | 1.00 | 0.00 |
| ATOM | 6 | CG | GLU | 1 | −3.618 | −11.246 | −10.519 | 1.00 | 0.00 |
| ATOM | 7 | CD | GLU | 1 | −2.651 | −10.942 | −11.683 | 1.00 | 0.00 |
| ATOM | 8 | OE1 | GLU | 1 | −2.284 | −11.843 | −12.488 | 1.00 | 0.00 |
| ATOM | 9 | OE2 | GLU | 1 | −2.208 | −9.755 | −11.837 | 1.00 | 0.00 |
| ATOM | 10 | HA | GLU | 1 | −0.924 | −10.890 | −9.553 | 1.00 | 0.00 |
| ATOM | 11 | HB3 | GLU | 1 | −3.695 | −11.949 | −8.632 | 1.00 | 0.00 |
| ATOM | 12 | HB2 | GLU | 1 | −2.598 | −12.922 | −9.689 | 1.00 | 0.00 |
| ATOM | 13 | HG3 | GLU | 1 | −3.903 | −10.236 | −10.221 | 1.00 | 0.00 |
| ATOM | 14 | HG2 | GLU | 1 | −4.469 | −11.799 | −10.917 | 1.00 | 0.00 |
| ATOM | 15 | H3 | GLU | 1 | −0.168 | −11.525 | −7.490 | 1.00 | 0.00 |
| ATOM | 16 | H2 | GLU | 1 | −1.618 | −12.171 | −7.040 | 1.00 | 0.00 |
| ATOM | 17 | H1 | GLU | 1 | −0.655 | −12.892 | −8.219 | 1.00 | 0.00 |
| ATOM | 18 | N | ILE | 2 | −1.888 | −8.827 | −9.134 | 1.00 | 0.00 |
| ATOM | 19 | CA | ILE | 2 | −1.972 | −7.456 | −8.725 | 1.00 | 0.00 |
| ATOM | 20 | C | ILE | 2 | −3.379 | −6.872 | −8.808 | 1.00 | 0.00 |
| ATOM | 21 | O | ILE | 2 | −4.052 | −6.960 | −9.835 | 1.00 | 0.00 |
| ATOM | 22 | CB | ILE | 2 | −0.911 | −6.543 | −9.428 | 1.00 | 0.00 |
| ATOM | 23 | CG1 | ILE | 2 | −0.773 | −5.051 | −8.961 | 1.00 | 0.00 |
| ATOM | 24 | CG2 | ILE | 2 | −1.013 | −6.471 | −10.902 | 1.00 | 0.00 |
| ATOM | 25 | CD1 | ILE | 2 | −0.034 | −4.993 | −7.593 | 1.00 | 0.00 |
| ATOM | 26 | HA | ILE | 2 | −1.734 | −7.456 | −7.662 | 1.00 | 0.00 |
| ATOM | 27 | HB | ILE | 2 | 0.097 | −6.857 | −9.159 | 1.00 | 0.00 |
| ATOM | 28 | HG13 | ILE | 2 | −1.776 | −4.626 | −8.935 | 1.00 | 0.00 |
| ATOM | 29 | HG12 | ILE | 2 | −0.139 | −4.577 | −9.709 | 1.00 | 0.00 |
| ATOM | 30 | HD11 | ILE | 2 | −0.028 | −3.916 | −7.429 | 1.00 | 0.00 |
| ATOM | 31 | HD12 | ILE | 2 | −0.539 | −5.667 | −6.901 | 1.00 | 0.00 |
| ATOM | 32 | HD13 | ILE | 2 | 1.002 | −5.325 | −7.651 | 1.00 | 0.00 |
| ATOM | 33 | HG21 | ILE | 2 | −1.618 | −5.630 | −11.239 | 1.00 | 0.00 |
| ATOM | 34 | HG22 | ILE | 2 | −0.038 | −6.285 | −11.352 | 1.00 | 0.00 |
| ATOM | 35 | HG23 | ILE | 2 | −1.358 | −7.431 | −11.288 | 1.00 | 0.00 |
| ATOM | 36 | H | ILE | 2 | −1.781 | −8.925 | −10.134 | 1.00 | 0.00 |
| ATOM | 37 | N | ILE | 3 | −3.840 | −6.339 | −7.663 | 1.00 | 0.00 |
| ATOM | 38 | CA | ILE | 3 | −5.204 | −5.732 | −7.516 | 1.00 | 0.00 |
| ATOM | 39 | C | ILE | 3 | −5.019 | −4.246 | −7.287 | 1.00 | 0.00 |
| ATOM | 40 | O | ILE | 3 | −3.951 | −3.757 | −6.877 | 1.00 | 0.00 |
| ATOM | 41 | CB | ILE | 3 | −5.883 | −6.376 | −6.275 | 1.00 | 0.00 |
| ATOM | 42 | CG1 | ILE | 3 | −5.633 | −7.891 | −6.085 | 1.00 | 0.00 |
| ATOM | 43 | CG2 | ILE | 3 | −7.322 | −6.171 | −6.442 | 1.00 | 0.00 |
| ATOM | 44 | CD1 | ILE | 3 | −6.016 | −8.858 | −7.293 | 1.00 | 0.00 |
| ATOM | 45 | HA | ILE | 3 | −5.820 | −5.923 | −8.395 | 1.00 | 0.00 |
| ATOM | 46 | HB | ILE | 3 | −5.403 | −5.805 | −5.480 | 1.00 | 0.00 |
| ATOM | 47 | HG13 | ILE | 3 | −6.141 | −8.194 | −5.170 | 1.00 | 0.00 |
| ATOM | 48 | HG12 | ILE | 3 | −4.559 | −7.988 | −5.921 | 1.00 | 0.00 |
| ATOM | 49 | HD11 | ILE | 3 | −7.088 | −9.049 | −7.356 | 1.00 | 0.00 |
| ATOM | 50 | HD12 | ILE | 3 | −5.651 | −8.499 | −8.255 | 1.00 | 0.00 |
| ATOM | 51 | HD13 | ILE | 3 | −5.587 | −9.853 | −7.174 | 1.00 | 0.00 |
| ATOM | 52 | HG21 | ILE | 3 | −7.575 | −5.114 | −6.351 | 1.00 | 0.00 |
| ATOM | 53 | HG22 | ILE | 3 | −7.575 | −6.473 | −7.457 | 1.00 | 0.00 |
| ATOM | 54 | HG23 | ILE | 3 | −7.861 | −6.751 | −5.692 | 1.00 | 0.00 |
| ATOM | 55 | H | ILE | 3 | −3.218 | −6.358 | −6.867 | 1.00 | 0.00 |
| ATOM | 56 | N | THR | 4 | −6.032 | −3.415 | −7.545 | 1.00 | 0.00 |
| ATOM | 57 | CA | THR | 4 | −6.017 | −1.964 | −7.336 | 1.00 | 0.00 |
| ATOM | 58 | C | THR | 4 | −7.298 | −1.383 | −6.650 | 1.00 | 0.00 |
| ATOM | 59 | O | THR | 4 | −8.390 | −1.898 | −6.770 | 1.00 | 0.00 |
| ATOM | 60 | CB | THR | 4 | −5.626 | −1.234 | −8.650 | 1.00 | 0.00 |
| ATOM | 61 | OG1 | THR | 4 | −5.528 | 0.173 | −8.392 | 1.00 | 0.00 |
| ATOM | 62 | CG2 | THR | 4 | −6.553 | −1.573 | −9.817 | 1.00 | 0.00 |
| ATOM | 63 | HA | THR | 4 | −5.176 | −1.601 | −6.744 | 1.00 | 0.00 |
| ATOM | 64 | HB | THR | 4 | −4.681 | −1.642 | −9.008 | 1.00 | 0.00 |
| ATOM | 65 | HG1 | THR | 4 | −4.827 | 0.288 | −7.746 | 1.00 | 0.00 |
| ATOM | 66 | HG23 | THR | 4 | −6.419 | −2.618 | −10.094 | 1.00 | 0.00 |
| ATOM | 67 | HG21 | THR | 4 | −7.541 | −1.296 | −9.450 | 1.00 | 0.00 |
| ATOM | 68 | HG22 | THR | 4 | −6.265 | −0.915 | −10.637 | 1.00 | 0.00 |
| ATOM | 69 | H | THR | 4 | −6.882 | −3.866 | −7.854 | 1.00 | 0.00 |
| ATOM | 70 | N | VAL | 5 | −7.078 | −0.334 | −5.805 | 1.00 | 0.00 |
| ATOM | 71 | CA | VAL | 5 | −8.079 | 0.272 | −4.967 | 1.00 | 0.00 |
| ATOM | 72 | C | VAL | 5 | −7.915 | 1.758 | −5.037 | 1.00 | 0.00 |
| ATOM | 73 | O | VAL | 5 | −6.878 | 2.222 | −5.500 | 1.00 | 0.00 |
| ATOM | 74 | CB | VAL | 5 | −8.124 | −0.258 | −3.525 | 1.00 | 0.00 |
| ATOM | 75 | CG1 | VAL | 5 | −9.472 | 0.069 | −2.940 | 1.00 | 0.00 |
| ATOM | 76 | CG2 | VAL | 5 | −7.981 | −1.773 | −3.413 | 1.00 | 0.00 |
| ATOM | 77 | HA | VAL | 5 | −9.037 | 0.143 | −5.471 | 1.00 | 0.00 |
| ATOM | 78 | HB | VAL | 5 | −7.316 | 0.138 | −2.910 | 1.00 | 0.00 |
| ATOM | 79 | HG11 | VAL | 5 | −9.843 | −0.646 | −2.206 | 1.00 | 0.00 |
| ATOM | 80 | HG12 | VAL | 5 | −9.430 | 1.031 | −2.431 | 1.00 | 0.00 |
| ATOM | 81 | HG13 | VAL | 5 | −10.278 | 0.102 | −3.674 | 1.00 | 0.00 |
| ATOM | 82 | HG21 | VAL | 5 | −7.041 | −2.147 | −3.818 | 1.00 | 0.00 |
| ATOM | 83 | HG22 | VAL | 5 | −8.043 | −2.088 | −2.371 | 1.00 | 0.00 |
| ATOM | 84 | HG23 | VAL | 5 | −8.842 | −2.216 | −3.916 | 1.00 | 0.00 |
| ATOM | 85 | H | VAL | 5 | −6.208 | 0.172 | −5.885 | 1.00 | 0.00 |
| ATOM | 86 | N | THR | 6 | −8.903 | 2.530 | −4.526 | 1.00 | 0.00 |
| ATOM | 87 | CA | THR | 6 | −8.696 | 3.963 | −4.281 | 1.00 | 0.00 |
| ATOM | 88 | C | THR | 6 | −8.796 | 4.364 | −2.797 | 1.00 | 0.00 |
| ATOM | 89 | O | THR | 6 | −9.555 | 3.757 | −2.003 | 1.00 | 0.00 |
| ATOM | 90 | CB | THR | 6 | −9.683 | 4.847 | −5.117 | 1.00 | 0.00 |
| ATOM | 91 | OG1 | THR | 6 | −11.027 | 4.631 | −4.829 | 1.00 | 0.00 |
| ATOM | 92 | CG2 | THR | 6 | −9.490 | 4.793 | −6.587 | 1.00 | 0.00 |
| ATOM | 93 | HA | THR | 6 | −7.701 | 4.228 | −4.639 | 1.00 | 0.00 |
| ATOM | 94 | HB | THR | 6 | −9.483 | 5.874 | −4.812 | 1.00 | 0.00 |
| ATOM | 95 | HG1 | THR | 6 | −11.298 | 4.043 | −5.538 | 1.00 | 0.00 |
| ATOM | 96 | HG23 | THR | 6 | −10.036 | 5.676 | −6.919 | 1.00 | 0.00 |
| ATOM | 97 | HG21 | THR | 6 | −8.465 | 4.963 | −6.915 | 1.00 | 0.00 |
| ATOM | 98 | HG22 | THR | 6 | −9.911 | 3.941 | −7.122 | 1.00 | 0.00 |
| ATOM | 99 | H | THR | 6 | −9.787 | 2.084 | −4.323 | 1.00 | 0.00 |
| ATOM | 100 | N | LEU | 7 | −8.208 | 5.505 | −2.374 | 1.00 | 0.00 |
| ATOM | 101 | CA | LEU | 7 | −8.300 | 6.003 | −0.931 | 1.00 | 0.00 |
| ATOM | 102 | C | LEU | 7 | −9.703 | 6.610 | −0.540 | 1.00 | 0.00 |
| ATOM | 103 | O | LEU | 7 | −9.785 | 7.467 | 0.322 | 1.00 | 0.00 |
| ATOM | 104 | CB | LEU | 7 | −7.166 | 6.934 | −0.676 | 1.00 | 0.00 |
| ATOM | 105 | CG | LEU | 7 | −6.280 | 6.481 | 0.469 | 1.00 | 0.00 |
| ATOM | 106 | CD1 | LEU | 7 | −4.965 | 7.108 | 0.542 | 1.00 | 0.00 |
| ATOM | 107 | CD2 | LEU | 7 | −6.961 | 6.548 | 1.804 | 1.00 | 0.00 |
| ATOM | 108 | HA | LEU | 7 | −8.289 | 5.097 | −0.324 | 1.00 | 0.00 |
| ATOM | 109 | HB3 | LEU | 7 | −7.532 | 7.952 | −0.549 | 1.00 | 0.00 |
| ATOM | 110 | HB2 | LEU | 7 | −6.527 | 6.943 | −1.558 | 1.00 | 0.00 |
| ATOM | 111 | HG | LEU | 7 | −6.098 | 5.430 | 0.243 | 1.00 | 0.00 |
| ATOM | 112 | HD21 | LEU | 7 | −7.223 | 7.570 | 2.078 | 1.00 | 0.00 |
| ATOM | 113 | HD22 | LEU | 7 | −7.881 | 5.964 | 1.806 | 1.00 | 0.00 |
| ATOM | 114 | HD23 | LEU | 7 | −6.405 | 6.003 | 2.566 | 1.00 | 0.00 |
| ATOM | 115 | HD11 | LEU | 7 | −4.516 | 6.784 | 1.481 | 1.00 | 0.00 |
| ATOM | 116 | HD12 | LEU | 7 | −4.366 | 6.798 | −0.314 | 1.00 | 0.00 |
| ATOM | 117 | HD13 | LEU | 7 | −5.017 | 8.196 | 0.592 | 1.00 | 0.00 |
| ATOM | 118 | H | LEU | 7 | −7.579 | 5.958 | −3.022 | 1.00 | 0.00 |
| ATOM | 119 | N | LYS | 8 | −10.798 | 6.141 | −1.206 | 1.00 | 0.00 |
| ATOM | 120 | CA | LYS | 8 | −12.067 | 6.874 | −1.289 | 1.00 | 0.00 |
| ATOM | 121 | C | LYS | 8 | −13.243 | 6.315 | −0.450 | 1.00 | 0.00 |
| ATOM | 122 | O | LYS | 8 | −13.954 | 7.156 | 0.165 | 1.00 | 0.00 |
| ATOM | 123 | CB | LYS | 8 | −12.535 | 7.001 | −2.796 | 1.00 | 0.00 |
| ATOM | 124 | CG | LYS | 8 | −13.900 | 7.797 | −2.939 | 1.00 | 0.00 |
| ATOM | 125 | CD | LYS | 8 | −14.236 | 8.091 | −4.386 | 1.00 | 0.00 |
| ATOM | 126 | CE | LYS | 8 | −15.756 | 8.231 | −4.448 | 1.00 | 0.00 |
| ATOM | 127 | NZ | LYS | 8 | −16.222 | 9.634 | −4.287 | 1.00 | 0.00 |
| ATOM | 128 | HA | LYS | 8 | −11.960 | 7.885 | −0.893 | 1.00 | 0.00 |
| ATOM | 129 | HB3 | LYS | 8 | −12.639 | 5.974 | −3.146 | 1.00 | 0.00 |
| ATOM | 130 | HB2 | LYS | 8 | −11.744 | 7.437 | −3.408 | 1.00 | 0.00 |
| ATOM | 131 | HG3 | LYS | 8 | −13.713 | 8.705 | −2.366 | 1.00 | 0.00 |
| ATOM | 132 | HG2 | LYS | 8 | −14.714 | 7.328 | −2.385 | 1.00 | 0.00 |
| ATOM | 133 | HD3 | LYS | 8 | −13.968 | 7.211 | −4.969 | 1.00 | 0.00 |
| ATOM | 134 | HD2 | LYS | 8 | −13.740 | 9.022 | −4.666 | 1.00 | 0.00 |
| ATOM | 135 | HE3 | LYS | 8 | −16.232 | 7.544 | −3.750 | 1.00 | 0.00 |
| ATOM | 136 | HE2 | LYS | 8 | −15.970 | 7.911 | −5.467 | 1.00 | 0.00 |
| ATOM | 137 | HZ1 | LYS | 8 | −17.157 | 9.880 | −4.577 | 1.00 | 0.00 |
| ATOM | 138 | HZ2 | LYS | 8 | −16.142 | 9.901 | −3.317 | 1.00 | 0.00 |
| ATOM | 139 | HZ3 | LYS | 8 | −15.671 | 10.336 | −4.759 | 1.00 | 0.00 |
| ATOM | 140 | H | LYS | 8 | −10.719 | 5.455 | −1.943 | 1.00 | 0.00 |
| ATOM | 141 | N | LYS | 9 | −13.310 | 4.974 | −0.365 | 1.00 | 0.00 |
| ATOM | 142 | CA | LYS | 9 | −14.492 | 4.339 | 0.266 | 1.00 | 0.00 |
| ATOM | 143 | C | LYS | 9 | −14.606 | 4.646 | 1.880 | 1.00 | 0.00 |
| ATOM | 144 | O | LYS | 9 | −15.740 | 4.611 | 2.449 | 1.00 | 0.00 |
| ATOM | 145 | CB | LYS | 9 | −14.435 | 2.857 | −0.059 | 1.00 | 0.00 |
| ATOM | 146 | CG | LYS | 9 | −15.629 | 2.040 | 0.457 | 1.00 | 0.00 |
| ATOM | 147 | CD | LYS | 9 | −16.910 | 2.295 | −0.392 | 1.00 | 0.00 |
| ATOM | 148 | CE | LYS | 9 | −18.056 | 1.334 | 0.107 | 1.00 | 0.00 |
| ATOM | 149 | NZ | LYS | 9 | −18.513 | 0.436 | −1.014 | 1.00 | 0.00 |
| ATOM | 150 | HA | LYS | 9 | −15.432 | 4.700 | −0.153 | 1.00 | 0.00 |
| ATOM | 151 | HB3 | LYS | 9 | −13.504 | 2.433 | 0.318 | 1.00 | 0.00 |
| ATOM | 152 | HB2 | LYS | 9 | −14.470 | 2.767 | −1.145 | 1.00 | 0.00 |
| ATOM | 153 | HG3 | LYS | 9 | −15.907 | 2.225 | 1.494 | 1.00 | 0.00 |
| ATOM | 154 | HG2 | LYS | 9 | −15.382 | 0.984 | 0.352 | 1.00 | 0.00 |
| ATOM | 155 | HD3 | LYS | 9 | −16.620 | 2.207 | −1.439 | 1.00 | 0.00 |
| ATOM | 156 | HD2 | LYS | 9 | −17.149 | 3.339 | −0.190 | 1.00 | 0.00 |
| ATOM | 157 | HE3 | LYS | 9 | −18.883 | 1.856 | 0.588 | 1.00 | 0.00 |
| ATOM | 158 | HE2 | LYS | 9 | −17.514 | 0.668 | 0.778 | 1.00 | 0.00 |
| ATOM | 159 | HZ1 | LYS | 9 | −18.874 | −0.487 | −0.825 | 1.00 | 0.00 |
| ATOM | 160 | HZ2 | LYS | 9 | −19.294 | 0.896 | −1.462 | 1.00 | 0.00 |
| ATOM | 161 | HZ3 | LYS | 9 | −17.832 | 0.280 | −1.741 | 1.00 | 0.00 |
| ATOM | 162 | H | LYS | 9 | −12.860 | 4.419 | −1.078 | 1.00 | 0.00 |
| ATOM | 163 | N | GLN | 10 | −13.466 | 4.928 | 2.522 | 1.00 | 0.00 |
| ATOM | 164 | CA | GLN | 10 | −13.228 | 5.051 | 3.998 | 1.00 | 0.00 |
| ATOM | 165 | C | GLN | 10 | −12.127 | 6.038 | 4.311 | 1.00 | 0.00 |
| ATOM | 166 | O | GLN | 10 | −11.343 | 6.364 | 3.373 | 1.00 | 0.00 |
| ATOM | 167 | CB | GLN | 10 | −13.057 | 3.740 | 4.644 | 1.00 | 0.00 |
| ATOM | 168 | CG | GLN | 10 | −14.312 | 2.899 | 4.565 | 1.00 | 0.00 |
| ATOM | 169 | CD | GLN | 10 | −14.281 | 1.747 | 5.513 | 1.00 | 0.00 |
| ATOM | 170 | OE1 | GLN | 10 | −13.599 | 0.808 | 5.369 | 1.00 | 0.00 |
| ATOM | 171 | NE2 | GLN | 10 | −15.112 | 1.616 | 6.543 | 1.00 | 0.00 |
| ATOM | 172 | HA | GLN | 10 | −14.189 | 5.452 | 4.319 | 1.00 | 0.00 |
| ATOM | 173 | HB3 | GLN | 10 | −12.882 | 3.913 | 5.706 | 1.00 | 0.00 |
| ATOM | 174 | HB2 | GLN | 10 | −12.241 | 3.203 | 4.163 | 1.00 | 0.00 |
| ATOM | 175 | HG3 | GLN | 10 | −14.626 | 2.572 | 3.574 | 1.00 | 0.00 |
| ATOM | 176 | HG2 | GLN | 10 | −15.182 | 3.505 | 4.818 | 1.00 | 0.00 |
| ATOM | 177 | HE22 | GLN | 10 | −15.827 | 2.315 | 6.681 | 1.00 | 0.00 |
| ATOM | 178 | HE21 | GLN | 10 | −15.159 | 0.847 | 7.197 | 1.00 | 0.00 |
| ATOM | 179 | H | GLN | 10 | −12.656 | 5.052 | 1.932 | 1.00 | 0.00 |
| ATOM | 180 | N | ASN | 11 | −12.087 | 6.551 | 5.504 | 1.00 | 0.00 |
| ATOM | 181 | CA | ASN | 11 | −11.076 | 7.481 | 6.104 | 1.00 | 0.00 |
| ATOM | 182 | C | ASN | 11 | −9.560 | 7.071 | 5.943 | 1.00 | 0.00 |
| ATOM | 183 | O | ASN | 11 | −8.708 | 7.895 | 6.250 | 1.00 | 0.00 |
| ATOM | 184 | CB | ASN | 11 | −11.483 | 7.680 | 7.608 | 1.00 | 0.00 |
| ATOM | 185 | CG | ASN | 11 | −11.051 | 9.005 | 8.218 | 1.00 | 0.00 |
| ATOM | 186 | OD1 | ASN | 11 | −10.224 | 9.129 | 9.099 | 1.00 | 0.00 |
| ATOM | 187 | ND2 | ASN | 11 | −11.726 | 10.035 | 7.786 | 1.00 | 0.00 |
| ATOM | 188 | HA | ASN | 11 | −11.309 | 8.403 | 5.571 | 1.00 | 0.00 |
| ATOM | 189 | HB3 | ASN | 11 | −10.960 | 6.886 | 8.143 | 1.00 | 0.00 |
| ATOM | 190 | HB2 | ASN | 11 | −12.546 | 7.554 | 7.816 | 1.00 | 0.00 |
| ATOM | 191 | HD22 | ASN | 11 | −12.490 | 10.019 | 7.125 | 1.00 | 0.00 |
| ATOM | 192 | HD21 | ASN | 11 | −11.506 | 10.899 | 8.259 | 1.00 | 0.00 |
| ATOM | 193 | H | ASN | 11 | −12.653 | 6.040 | 6.166 | 1.00 | 0.00 |
| ATOM | 194 | N | GLY | 12 | −9.264 | 5.825 | 5.495 | 1.00 | 0.00 |
| ATOM | 195 | CA | GLY | 12 | −7.935 | 5.201 | 5.327 | 1.00 | 0.00 |
| ATOM | 196 | C | GLY | 12 | −7.878 | 3.846 | 4.636 | 1.00 | 0.00 |
| ATOM | 197 | O | GLY | 12 | −8.818 | 3.448 | 4.008 | 1.00 | 0.00 |
| ATOM | 198 | HA3 | GLY | 12 | −7.594 | 5.199 | 6.363 | 1.00 | 0.00 |
| ATOM | 199 | HA2 | GLY | 12 | −7.256 | 5.897 | 4.836 | 1.00 | 0.00 |
| ATOM | 200 | H | GLY | 12 | −10.069 | 5.244 | 5.312 | 1.00 | 0.00 |
| ATOM | 201 | N | MET | 13 | −6.717 | 3.252 | 4.778 | 1.00 | 0.00 |
| ATOM | 202 | CA | MET | 13 | −6.250 | 2.010 | 4.024 | 1.00 | 0.00 |
| ATOM | 203 | C | MET | 13 | −6.666 | 0.762 | 4.555 | 1.00 | 0.00 |
| ATOM | 204 | O | MET | 13 | −7.206 | −0.012 | 3.766 | 1.00 | 0.00 |
| ATOM | 205 | CB | MET | 13 | −4.689 | 1.912 | 4.023 | 1.00 | 0.00 |
| ATOM | 206 | CG | MET | 13 | −4.213 | 3.211 | 3.412 | 1.00 | 0.00 |
| ATOM | 207 | SD | MET | 13 | −4.623 | 3.442 | 1.689 | 1.00 | 0.00 |
| ATOM | 208 | CE | MET | 13 | −3.166 | 4.295 | 1.094 | 1.00 | 0.00 |
| ATOM | 209 | HA | MET | 13 | −6.686 | 2.057 | 3.027 | 1.00 | 0.00 |
| ATOM | 210 | HB3 | MET | 13 | −4.388 | 1.016 | 3.482 | 1.00 | 0.00 |
| ATOM | 211 | HB2 | MET | 13 | −4.356 | 1.870 | 5.060 | 1.00 | 0.00 |
| ATOM | 212 | HG3 | MET | 13 | −3.136 | 3.261 | 3.571 | 1.00 | 0.00 |
| ATOM | 213 | HG2 | MET | 13 | −4.598 | 4.074 | 3.956 | 1.00 | 0.00 |
| ATOM | 214 | HE1 | MET | 13 | −2.301 | 3.645 | 0.965 | 1.00 | 0.00 |
| ATOM | 215 | HE2 | MET | 13 | −3.307 | 4.657 | 0.077 | 1.00 | 0.00 |
| ATOM | 216 | HE3 | MET | 13 | −2.759 | 5.139 | 1.652 | 1.00 | 0.00 |
| ATOM | 217 | H | MET | 13 | −5.985 | 3.671 | 5.333 | 1.00 | 0.00 |
| ATOM | 218 | N | GLY | 14 | −6.640 | 0.492 | 5.886 | 1.00 | 0.00 |
| ATOM | 219 | CA | GLY | 14 | −6.962 | −0.853 | 6.361 | 1.00 | 0.00 |
| ATOM | 220 | C | GLY | 14 | −6.124 | −1.993 | 5.859 | 1.00 | 0.00 |
| ATOM | 221 | O | GLY | 14 | −6.793 | −2.985 | 5.356 | 1.00 | 0.00 |
| ATOM | 222 | HA3 | GLY | 14 | −7.975 | −1.126 | 6.067 | 1.00 | 0.00 |
| ATOM | 223 | HA2 | GLY | 14 | −6.953 | −0.837 | 7.450 | 1.00 | 0.00 |
| ATOM | 224 | H | GLY | 14 | −6.170 | 1.144 | 6.498 | 1.00 | 0.00 |
| ATOM | 225 | N | LEU | 15 | −4.824 | −1.810 | 5.937 | 1.00 | 0.00 |
| ATOM | 226 | CA | LEU | 15 | −3.709 | −2.666 | 5.501 | 1.00 | 0.00 |
| ATOM | 227 | C | LEU | 15 | −2.453 | −2.821 | 6.467 | 1.00 | 0.00 |
| ATOM | 228 | O | LEU | 15 | −2.061 | −1.875 | 7.160 | 1.00 | 0.00 |
| ATOM | 229 | CB | LEU | 15 | −3.318 | −2.099 | 4.127 | 1.00 | 0.00 |
| ATOM | 230 | CG | LEU | 15 | −3.886 | −2.911 | 2.989 | 1.00 | 0.00 |
| ATOM | 231 | CD1 | LEU | 15 | −3.624 | −2.054 | 1.743 | 1.00 | 0.00 |
| ATOM | 232 | CD2 | LEU | 15 | −3.359 | −4.348 | 2.713 | 1.00 | 0.00 |
| ATOM | 233 | HA | LEU | 15 | −4.190 | −3.635 | 5.374 | 1.00 | 0.00 |
| ATOM | 234 | HB3 | LEU | 15 | −2.249 | −2.207 | 3.939 | 1.00 | 0.00 |
| ATOM | 235 | HB2 | LEU | 15 | −3.484 | −1.029 | 3.997 | 1.00 | 0.00 |
| ATOM | 236 | HG | LEU | 15 | −4.945 | −3.031 | 3.220 | 1.00 | 0.00 |
| ATOM | 237 | HD21 | LEU | 15 | −3.755 | −4.874 | 1.846 | 1.00 | 0.00 |
| ATOM | 238 | HD22 | LEU | 15 | −2.297 | −4.240 | 2.494 | 1.00 | 0.00 |
| ATOM | 239 | HD23 | LEU | 15 | −3.505 | −4.893 | 3.646 | 1.00 | 0.00 |
| ATOM | 240 | HD11 | LEU | 15 | −2.578 | −2.101 | 1.442 | 1.00 | 0.00 |
| ATOM | 241 | HD12 | LEU | 15 | −4.113 | −2.433 | 0.845 | 1.00 | 0.00 |
| ATOM | 242 | HD13 | LEU | 15 | −3.753 | −0.989 | 1.938 | 1.00 | 0.00 |
| ATOM | 243 | H | LEU | 15 | −4.536 | −0.991 | 6.452 | 1.00 | 0.00 |
| ATOM | 244 | N | SER | 16 | −1.824 | −4.002 | 6.411 | 1.00 | 0.00 |
| ATOM | 245 | CA | SER | 16 | −0.600 | −4.325 | 7.158 | 1.00 | 0.00 |
| ATOM | 246 | C | SER | 16 | 0.481 | −4.793 | 6.160 | 1.00 | 0.00 |
| ATOM | 247 | O | SER | 16 | 0.449 | −5.846 | 5.555 | 1.00 | 0.00 |
| ATOM | 248 | CB | SER | 16 | −0.984 | −5.406 | 8.122 | 1.00 | 0.00 |
| ATOM | 249 | OG | SER | 16 | −1.695 | −4.859 | 9.254 | 1.00 | 0.00 |
| ATOM | 250 | HA | SER | 16 | −0.284 | −3.425 | 7.684 | 1.00 | 0.00 |
| ATOM | 251 | HB3 | SER | 16 | −0.109 | −5.889 | 8.557 | 1.00 | 0.00 |
| ATOM | 252 | HB2 | SER | 16 | −1.528 | −6.193 | 7.601 | 1.00 | 0.00 |
| ATOM | 253 | HG | SER | 16 | −2.106 | −5.573 | 9.746 | 1.00 | 0.00 |
| ATOM | 254 | H | SER | 16 | −2.295 | −4.684 | 5.834 | 1.00 | 0.00 |
| ATOM | 255 | N | ILE | 17 | 1.596 | −4.038 | 6.077 | 1.00 | 0.00 |
| ATOM | 256 | CA | ILE | 17 | 2.869 | −4.398 | 5.406 | 1.00 | 0.00 |
| ATOM | 257 | C | ILE | 17 | 3.903 | −4.862 | 6.407 | 1.00 | 0.00 |
| ATOM | 258 | O | ILE | 17 | 3.782 | −4.551 | 7.579 | 1.00 | 0.00 |
| ATOM | 259 | CB | ILE | 17 | 3.277 | −3.095 | 4.646 | 1.00 | 0.00 |
| ATOM | 260 | CG1 | ILE | 17 | 3.571 | −1.837 | 5.634 | 1.00 | 0.00 |
| ATOM | 261 | CG2 | ILE | 17 | 2.229 | −2.773 | 3.582 | 1.00 | 0.00 |
| ATOM | 262 | CD1 | ILE | 17 | 4.355 | −0.694 | 4.998 | 1.00 | 0.00 |
| ATOM | 263 | HA | ILE | 17 | 2.791 | −5.116 | 4.590 | 1.00 | 0.00 |
| ATOM | 264 | HB | ILE | 17 | 4.210 | −3.385 | 4.163 | 1.00 | 0.00 |
| ATOM | 265 | HG13 | ILE | 17 | 4.065 | −2.203 | 6.534 | 1.00 | 0.00 |
| ATOM | 266 | HG12 | ILE | 17 | 2.622 | −1.386 | 5.924 | 1.00 | 0.00 |
| ATOM | 267 | HD11 | ILE | 17 | 3.704 | −0.221 | 4.263 | 1.00 | 0.00 |
| ATOM | 268 | HD12 | ILE | 17 | 4.613 | 0.033 | 5.767 | 1.00 | 0.00 |
| ATOM | 269 | HD13 | ILE | 17 | 5.219 | −1.116 | 4.483 | 1.00 | 0.00 |
| ATOM | 270 | HG21 | ILE | 17 | 2.697 | −2.317 | 2.710 | 1.00 | 0.00 |
| ATOM | 271 | HG22 | ILE | 17 | 1.700 | −3.629 | 3.163 | 1.00 | 0.00 |
| ATOM | 272 | HG23 | ILE | 17 | 1.507 | −2.061 | 3.983 | 1.00 | 0.00 |
| ATOM | 273 | H | ILE | 17 | 1.713 | −3.219 | 6.655 | 1.00 | 0.00 |
| ATOM | 274 | N | VAL | 18 | 4.917 | −5.698 | 5.971 | 1.00 | 0.00 |
| ATOM | 275 | CA | VAL | 18 | 5.885 | −6.376 | 6.847 | 1.00 | 0.00 |
| ATOM | 276 | C | VAL | 18 | 7.348 | −6.313 | 6.374 | 1.00 | 0.00 |
| ATOM | 277 | O | VAL | 18 | 8.155 | −7.136 | 6.732 | 1.00 | 0.00 |
| ATOM | 278 | CB | VAL | 18 | 5.338 | −7.827 | 7.072 | 1.00 | 0.00 |
| ATOM | 279 | CG1 | VAL | 18 | 5.405 | −8.581 | 5.698 | 1.00 | 0.00 |
| ATOM | 280 | CG2 | VAL | 18 | 5.908 | −8.599 | 8.253 | 1.00 | 0.00 |
| ATOM | 281 | HA | VAL | 18 | 5.914 | −5.882 | 7.818 | 1.00 | 0.00 |
| ATOM | 282 | HB | VAL | 18 | 4.263 | −7.781 | 7.244 | 1.00 | 0.00 |
| ATOM | 283 | HG11 | VAL | 18 | 5.155 | −9.637 | 5.798 | 1.00 | 0.00 |
| ATOM | 284 | HG12 | VAL | 18 | 4.813 | −8.096 | 4.921 | 1.00 | 0.00 |
| ATOM | 285 | HG13 | VAL | 18 | 6.388 | −8.533 | 5.230 | 1.00 | 0.00 |
| ATOM | 286 | HG21 | VAL | 18 | 6.979 | −8.752 | 8.128 | 1.00 | 0.00 |
| ATOM | 287 | HG22 | VAL | 18 | 5.748 | −7.938 | 9.104 | 1.00 | 0.00 |
| ATOM | 288 | HG23 | VAL | 18 | 5.405 | −9.565 | 8.294 | 1.00 | 0.00 |
| ATOM | 289 | H | VAL | 18 | 4.983 | −5.973 | 5.001 | 1.00 | 0.00 |
| ATOM | 290 | N | ALA | 19 | 7.707 | −5.278 | 5.475 | 1.00 | 0.00 |
| ATOM | 291 | CA | ALA | 19 | 9.103 | −5.225 | 4.957 | 1.00 | 0.00 |
| ATOM | 292 | C | ALA | 19 | 9.597 | −3.852 | 5.013 | 1.00 | 0.00 |
| ATOM | 293 | O | ALA | 19 | 8.862 | −2.884 | 5.302 | 1.00 | 0.00 |
| ATOM | 294 | CB | ALA | 19 | 9.171 | −5.799 | 3.520 | 1.00 | 0.00 |
| ATOM | 295 | HA | ALA | 19 | 9.697 | −5.921 | 5.548 | 1.00 | 0.00 |
| ATOM | 296 | HB1 | ALA | 19 | 8.978 | −6.864 | 3.391 | 1.00 | 0.00 |
| ATOM | 297 | HB2 | ALA | 19 | 8.489 | −5.175 | 2.942 | 1.00 | 0.00 |
| ATOM | 298 | HB3 | ALA | 19 | 10.196 | −5.695 | 3.165 | 1.00 | 0.00 |
| ATOM | 299 | H | ALA | 19 | 7.135 | −4.462 | 5.312 | 1.00 | 0.00 |
| ATOM | 300 | N | ALA | 20 | 10.879 | −3.647 | 4.831 | 1.00 | 0.00 |
| ATOM | 301 | CA | ALA | 20 | 11.471 | −2.313 | 5.119 | 1.00 | 0.00 |
| ATOM | 302 | C | ALA | 20 | 12.828 | −2.340 | 4.427 | 1.00 | 0.00 |
| ATOM | 303 | O | ALA | 20 | 13.570 | −3.256 | 4.742 | 1.00 | 0.00 |
| ATOM | 304 | CB | ALA | 20 | 11.616 | −2.226 | 6.677 | 1.00 | 0.00 |
| ATOM | 305 | HA | ALA | 20 | 10.836 | −1.495 | 4.778 | 1.00 | 0.00 |
| ATOM | 306 | HB1 | ALA | 20 | 10.649 | −2.385 | 7.154 | 1.00 | 0.00 |
| ATOM | 307 | HB2 | ALA | 20 | 12.269 | −3.009 | 7.061 | 1.00 | 0.00 |
| ATOM | 308 | HB3 | ALA | 20 | 12.007 | −1.238 | 6.920 | 1.00 | 0.00 |
| ATOM | 309 | H | ALA | 20 | 11.413 | −4.251 | 4.223 | 1.00 | 0.00 |
| ATOM | 310 | N | LYS | 21 | 12.984 | −1.473 | 3.404 | 1.00 | 0.00 |
| ATOM | 311 | CA | LYS | 21 | 14.202 | −1.341 | 2.623 | 1.00 | 0.00 |
| ATOM | 312 | C | LYS | 21 | 15.500 | −1.327 | 3.465 | 1.00 | 0.00 |
| ATOM | 313 | O | LYS | 21 | 15.695 | −0.351 | 4.212 | 1.00 | 0.00 |
| ATOM | 314 | CB | LYS | 21 | 14.132 | −0.110 | 1.711 | 1.00 | 0.00 |
| ATOM | 315 | CG | LYS | 21 | 15.298 | 0.020 | 0.769 | 1.00 | 0.00 |
| ATOM | 316 | CD | LYS | 21 | 14.968 | 1.066 | −0.318 | 1.00 | 0.00 |
| ATOM | 317 | CE | LYS | 21 | 15.947 | 1.018 | −1.473 | 1.00 | 0.00 |
| ATOM | 318 | NZ | LYS | 21 | 17.270 | 1.742 | −1.264 | 1.00 | 0.00 |
| ATOM | 319 | HA | LYS | 21 | 14.287 | −2.242 | 2.018 | 1.00 | 0.00 |
| ATOM | 320 | HB3 | LYS | 21 | 14.133 | 0.715 | 2.421 | 1.00 | 0.00 |
| ATOM | 321 | HB2 | LYS | 21 | 13.210 | −0.039 | 1.134 | 1.00 | 0.00 |
| ATOM | 322 | HG3 | LYS | 21 | 15.617 | −0.903 | 0.285 | 1.00 | 0.00 |
| ATOM | 323 | HG2 | LYS | 21 | 16.036 | 0.533 | 1.386 | 1.00 | 0.00 |
| ATOM | 324 | HD3 | LYS | 21 | 14.938 | 2.070 | 0.105 | 1.00 | 0.00 |
| ATOM | 325 | HD2 | LYS | 21 | 13.957 | 0.899 | −0.689 | 1.00 | 0.00 |
| ATOM | 326 | HE3 | LYS | 21 | 15.397 | 1.492 | −2.286 | 1.00 | 0.00 |
| ATOM | 327 | HE2 | LYS | 21 | 16.238 | −0.011 | −1.681 | 1.00 | 0.00 |
| ATOM | 328 | HZ1 | LYS | 21 | 17.655 | 2.095 | −2.130 | 1.00 | 0.00 |
| ATOM | 329 | HZ2 | LYS | 21 | 17.156 | 2.571 | −0.698 | 1.00 | 0.00 |
| ATOM | 330 | HZ3 | LYS | 21 | 17.947 | 1.164 | −0.789 | 1.00 | 0.00 |
| ATOM | 331 | H | LYS | 21 | 12.211 | −0.840 | 3.245 | 1.00 | 0.00 |
| ATOM | 332 | N | GLY | 22 | 16.356 | −2.330 | 3.324 | 1.00 | 0.00 |
| ATOM | 333 | CA | GLY | 22 | 17.690 | −2.347 | 3.838 | 1.00 | 0.00 |
| ATOM | 334 | C | GLY | 22 | 18.388 | −3.684 | 3.795 | 1.00 | 0.00 |
| ATOM | 335 | O | GLY | 22 | 19.035 | −4.075 | 2.796 | 1.00 | 0.00 |
| ATOM | 336 | HA3 | GLY | 22 | 17.599 | −2.129 | 4.901 | 1.00 | 0.00 |
| ATOM | 337 | HA2 | GLY | 22 | 18.270 | −1.602 | 3.294 | 1.00 | 0.00 |
| ATOM | 338 | H | GLY | 22 | 15.996 | −3.148 | 2.852 | 1.00 | 0.00 |
| ATOM | 339 | N | ALA | 23 | 18.301 | −4.434 | 4.922 | 1.00 | 0.00 |
| ATOM | 340 | CA | ALA | 23 | 18.623 | −5.860 | 5.064 | 1.00 | 0.00 |
| ATOM | 341 | C | ALA | 23 | 20.095 | −6.279 | 4.694 | 1.00 | 0.00 |
| ATOM | 342 | O | ALA | 23 | 20.282 | −7.390 | 4.235 | 1.00 | 0.00 |
| ATOM | 343 | CB | ALA | 23 | 17.512 | −6.720 | 4.415 | 1.00 | 0.00 |
| ATOM | 344 | HA | ALA | 23 | 18.517 | −5.968 | 6.143 | 1.00 | 0.00 |
| ATOM | 345 | HB1 | ALA | 23 | 17.532 | −6.471 | 3.355 | 1.00 | 0.00 |
| ATOM | 346 | HB2 | ALA | 23 | 17.570 | −7.785 | 4.642 | 1.00 | 0.00 |
| ATOM | 347 | HB3 | ALA | 23 | 16.593 | −6.371 | 4.886 | 1.00 | 0.00 |
| ATOM | 348 | H | ALA | 23 | 17.665 | −4.059 | 5.609 | 1.00 | 0.00 |
| ATOM | 349 | N | GLY | 24 | 21.019 | −5.351 | 4.901 | 1.00 | 0.00 |
| ATOM | 350 | CA | GLY | 24 | 22.362 | −5.411 | 4.486 | 1.00 | 0.00 |
| ATOM | 351 | C | GLY | 24 | 22.619 | −5.586 | 3.012 | 1.00 | 0.00 |
| ATOM | 352 | O | GLY | 24 | 23.595 | −6.161 | 2.675 | 1.00 | 0.00 |
| ATOM | 353 | HA3 | GLY | 24 | 22.878 | −6.222 | 5.002 | 1.00 | 0.00 |
| ATOM | 354 | HA2 | GLY | 24 | 22.839 | −4.504 | 4.856 | 1.00 | 0.00 |
| ATOM | 355 | H | GLY | 24 | 20.583 | −4.443 | 4.972 | 1.00 | 0.00 |
| ATOM | 356 | N | GLN | 25 | 21.678 | −5.186 | 2.114 | 1.00 | 0.00 |
| ATOM | 357 | CA | GLN | 25 | 21.763 | −5.246 | 0.643 | 1.00 | 0.00 |
| ATOM | 358 | C | GLN | 25 | 21.217 | −3.990 | −0.036 | 1.00 | 0.00 |
| ATOM | 359 | O | GLN | 25 | 21.115 | −3.950 | −1.263 | 1.00 | 0.00 |
| ATOM | 360 | CB | GLN | 25 | 21.062 | −6.499 | 0.002 | 1.00 | 0.00 |
| ATOM | 361 | CG | GLN | 25 | 21.723 | −7.798 | 0.384 | 1.00 | 0.00 |
| ATOM | 362 | CD | GLN | 25 | 20.919 | −8.988 | −0.209 | 1.00 | 0.00 |
| ATOM | 363 | OE1 | GLN | 25 | 20.005 | −9.576 | 0.380 | 1.00 | 0.00 |
| ATOM | 364 | NE2 | GLN | 25 | 21.227 | −9.387 | −1.411 | 1.00 | 0.00 |
| ATOM | 365 | HA | GLN | 25 | 22.806 | −5.341 | 0.345 | 1.00 | 0.00 |
| ATOM | 366 | HB3 | GLN | 25 | 21.189 | −6.474 | −1.080 | 1.00 | 0.00 |
| ATOM | 367 | HB2 | GLN | 25 | 20.017 | −6.629 | 0.287 | 1.00 | 0.00 |
| ATOM | 368 | HG3 | GLN | 25 | 21.661 | −8.025 | 1.448 | 1.00 | 0.00 |
| ATOM | 369 | HG2 | GLN | 25 | 22.736 | −7.690 | −0.003 | 1.00 | 0.00 |
| ATOM | 370 | HE22 | GLN | 25 | 21.902 | −8.891 | −1.974 | 1.00 | 0.00 |
| ATOM | 371 | HE21 | GLN | 25 | 20.933 | −10.311 | −1.693 | 1.00 | 0.00 |
| ATOM | 372 | H | GLN | 25 | 20.812 | −4.813 | 2.475 | 1.00 | 0.00 |
| ATOM | 373 | N | ASP | 26 | 20.704 | −3.007 | 0.749 | 1.00 | 0.00 |
| ATOM | 374 | CA | ASP | 26 | 19.911 | −1.753 | 0.375 | 1.00 | 0.00 |
| ATOM | 375 | C | ASP | 26 | 18.654 | −1.998 | −0.475 | 1.00 | 0.00 |
| ATOM | 376 | O | ASP | 26 | 18.229 | −1.203 | −1.340 | 1.00 | 0.00 |
| ATOM | 377 | CB | ASP | 26 | 20.828 | −0.618 | −0.166 | 1.00 | 0.00 |
| ATOM | 378 | CG | ASP | 26 | 20.086 | 0.723 | −0.234 | 1.00 | 0.00 |
| ATOM | 379 | OD1 | ASP | 26 | 20.194 | 1.362 | −1.256 | 1.00 | 0.00 |
| ATOM | 380 | OD2 | ASP | 26 | 19.461 | 1.180 | 0.720 | 1.00 | 0.00 |
| ATOM | 381 | HA | ASP | 26 | 19.614 | −1.316 | 1.329 | 1.00 | 0.00 |
| ATOM | 382 | HB3 | ASP | 26 | 21.096 | −0.821 | −1.201 | 1.00 | 0.00 |
| ATOM | 383 | HB2 | ASP | 26 | 21.737 | −0.547 | 0.433 | 1.00 | 0.00 |
| ATOM | 384 | H | ASP | 26 | 20.661 | −3.231 | 1.733 | 1.00 | 0.00 |
| ATOM | 385 | N | LYS | 27 | 17.962 | −3.087 | −0.179 | 1.00 | 0.00 |
| ATOM | 386 | CA | LYS | 27 | 16.715 | −3.567 | −0.839 | 1.00 | 0.00 |
| ATOM | 387 | C | LYS | 27 | 15.868 | −4.358 | 0.249 | 1.00 | 0.00 |
| ATOM | 388 | O | LYS | 27 | 16.374 | −4.800 | 1.269 | 1.00 | 0.00 |
| ATOM | 389 | CB | LYS | 27 | 16.975 | −4.412 | −2.127 | 1.00 | 0.00 |
| ATOM | 390 | CG | LYS | 27 | 17.751 | −5.721 | −2.020 | 1.00 | 0.00 |
| ATOM | 391 | CD | LYS | 27 | 18.147 | −6.305 | −3.377 | 1.00 | 0.00 |
| ATOM | 392 | CE | LYS | 27 | 18.652 | −7.695 | −3.331 | 1.00 | 0.00 |
| ATOM | 393 | NZ | LYS | 27 | 18.903 | −8.235 | −4.655 | 1.00 | 0.00 |
| ATOM | 394 | HA | LYS | 27 | 16.343 | −2.604 | −1.190 | 1.00 | 0.00 |
| ATOM | 395 | HB3 | LYS | 27 | 17.539 | −3.721 | −2.754 | 1.00 | 0.00 |
| ATOM | 396 | HB2 | LYS | 27 | 16.035 | −4.578 | −2.653 | 1.00 | 0.00 |
| ATOM | 397 | HG3 | LYS | 27 | 17.203 | −6.481 | −1.463 | 1.00 | 0.00 |
| ATOM | 398 | HG2 | LYS | 27 | 18.654 | −5.486 | −1.457 | 1.00 | 0.00 |
| ATOM | 399 | HD3 | LYS | 27 | 18.837 | −5.598 | −3.839 | 1.00 | 0.00 |
| ATOM | 400 | HD2 | LYS | 27 | 17.199 | −6.302 | −3.914 | 1.00 | 0.00 |
| ATOM | 401 | HE3 | LYS | 27 | 17.882 | −8.305 | −2.858 | 1.00 | 0.00 |
| ATOM | 402 | HE2 | LYS | 27 | 19.519 | −7.723 | −2.670 | 1.00 | 0.00 |
| ATOM | 403 | HZ1 | LYS | 27 | 18.961 | −9.232 | −4.499 | 1.00 | 0.00 |
| ATOM | 404 | HZ2 | LYS | 27 | 18.119 | −8.117 | −5.281 | 1.00 | 0.00 |
| ATOM | 405 | HZ3 | LYS | 27 | 19.857 | −8.048 | −4.930 | 1.00 | 0.00 |
| ATOM | 406 | H | LYS | 27 | 18.208 | −3.569 | 0.674 | 1.00 | 0.00 |
| ATOM | 407 | N | LEU | 28 | 14.552 | −4.392 | 0.064 | 1.00 | 0.00 |
| ATOM | 408 | CA | LEU | 28 | 13.642 | −5.389 | 0.704 | 1.00 | 0.00 |
| ATOM | 409 | C | LEU | 28 | 12.224 | −5.242 | 0.281 | 1.00 | 0.00 |
| ATOM | 410 | O | LEU | 28 | 11.468 | −6.156 | 0.557 | 1.00 | 0.00 |
| ATOM | 411 | CB | LEU | 28 | 13.615 | −5.298 | 2.243 | 1.00 | 0.00 |
| ATOM | 412 | CG | LEU | 28 | 13.762 | −6.601 | 3.061 | 1.00 | 0.00 |
| ATOM | 413 | CD1 | LEU | 28 | 13.769 | −6.332 | 4.558 | 1.00 | 0.00 |
| ATOM | 414 | CD2 | LEU | 28 | 12.880 | −7.814 | 2.633 | 1.00 | 0.00 |
| ATOM | 415 | HA | LEU | 28 | 13.791 | −6.413 | 0.360 | 1.00 | 0.00 |
| ATOM | 416 | HB3 | LEU | 28 | 12.681 | −4.834 | 2.560 | 1.00 | 0.00 |
| ATOM | 417 | HB2 | LEU | 28 | 14.522 | −4.770 | 2.537 | 1.00 | 0.00 |
| ATOM | 418 | HG | LEU | 28 | 14.789 | −6.932 | 2.904 | 1.00 | 0.00 |
| ATOM | 419 | HD21 | LEU | 28 | 11.826 | −7.553 | 2.723 | 1.00 | 0.00 |
| ATOM | 420 | HD22 | LEU | 28 | 13.042 | −8.131 | 1.601 | 1.00 | 0.00 |
| ATOM | 421 | HD23 | LEU | 28 | 13.202 | −8.708 | 3.166 | 1.00 | 0.00 |
| ATOM | 422 | HD11 | LEU | 28 | 14.630 | −5.695 | 4.755 | 1.00 | 0.00 |
| ATOM | 423 | HD12 | LEU | 28 | 12.901 | −5.774 | 4.910 | 1.00 | 0.00 |
| ATOM | 424 | HD13 | LEU | 28 | 13.839 | −7.259 | 5.126 | 1.00 | 0.00 |
| ATOM | 425 | H | LEU | 28 | 14.113 | −3.791 | −0.618 | 1.00 | 0.00 |
| ATOM | 426 | N | GLY | 29 | 11.852 | −4.134 | −0.238 | 1.00 | 0.00 |
| ATOM | 427 | CA | GLY | 29 | 10.507 | −3.927 | −0.712 | 1.00 | 0.00 |
| ATOM | 428 | C | GLY | 29 | 9.626 | −3.428 | 0.423 | 1.00 | 0.00 |
| ATOM | 429 | O | GLY | 29 | 10.012 | −3.032 | 1.558 | 1.00 | 0.00 |
| ATOM | 430 | HA3 | GLY | 29 | 10.105 | −4.844 | −1.144 | 1.00 | 0.00 |
| ATOM | 431 | HA2 | GLY | 29 | 10.397 | −3.214 | −1.528 | 1.00 | 0.00 |
| ATOM | 432 | H | GLY | 29 | 12.489 | −3.358 | −0.347 | 1.00 | 0.00 |
| ATOM | 433 | N | ILE | 30 | 8.381 | −3.379 | 0.058 | 1.00 | 0.00 |
| ATOM | 434 | CA | ILE | 30 | 7.208 | −3.174 | 0.922 | 1.00 | 0.00 |
| ATOM | 435 | C | ILE | 30 | 6.187 | −4.289 | 0.718 | 1.00 | 0.00 |
| ATOM | 436 | O | ILE | 30 | 5.051 | −4.077 | 0.291 | 1.00 | 0.00 |
| ATOM | 437 | CB | ILE | 30 | 6.587 | −1.752 | 0.775 | 1.00 | 0.00 |
| ATOM | 438 | CG1 | ILE | 30 | 6.545 | −1.274 | −0.683 | 1.00 | 0.00 |
| ATOM | 439 | CG2 | ILE | 30 | 7.198 | −0.777 | 1.710 | 1.00 | 0.00 |
| ATOM | 440 | CD1 | ILE | 30 | 6.157 | 0.239 | −0.956 | 1.00 | 0.00 |
| ATOM | 441 | HA | ILE | 30 | 7.496 | −3.307 | 1.965 | 1.00 | 0.00 |
| ATOM | 442 | HB | ILE | 30 | 5.579 | −1.872 | 1.174 | 1.00 | 0.00 |
| ATOM | 443 | HG13 | ILE | 30 | 5.882 | −1.904 | −1.276 | 1.00 | 0.00 |
| ATOM | 444 | HG12 | ILE | 30 | 7.559 | −1.332 | −1.079 | 1.00 | 0.00 |
| ATOM | 445 | HD11 | ILE | 30 | 6.094 | 0.418 | −2.030 | 1.00 | 0.00 |
| ATOM | 446 | HD12 | ILE | 30 | 5.201 | 0.391 | −0.454 | 1.00 | 0.00 |
| ATOM | 447 | HD13 | ILE | 30 | 7.010 | 0.767 | −0.530 | 1.00 | 0.00 |
| ATOM | 448 | HG21 | ILE | 30 | 6.649 | 0.164 | 1.737 | 1.00 | 0.00 |
| ATOM | 449 | HG22 | ILE | 30 | 7.314 | −1.212 | 2.702 | 1.00 | 0.00 |
| ATOM | 450 | HG23 | ILE | 30 | 8.203 | −0.531 | 1.368 | 1.00 | 0.00 |
| ATOM | 451 | H | ILE | 30 | 8.269 | −3.586 | −0.924 | 1.00 | 0.00 |
| ATOM | 452 | N | TYR | 31 | 6.635 | −5.516 | 1.035 | 1.00 | 0.00 |
| ATOM | 453 | CA | TYR | 31 | 5.820 | −6.773 | 1.012 | 1.00 | 0.00 |
| ATOM | 454 | C | TYR | 31 | 4.671 | −6.760 | 2.006 | 1.00 | 0.00 |
| ATOM | 455 | O | TYR | 31 | 4.826 | −6.146 | 3.092 | 1.00 | 0.00 |
| ATOM | 456 | CB | TYR | 31 | 6.790 | −7.962 | 1.341 | 1.00 | 0.00 |
| ATOM | 457 | CG | TYR | 31 | 6.420 | −9.375 | 1.045 | 1.00 | 0.00 |
| ATOM | 458 | CD1 | TYR | 31 | 6.905 | −10.120 | −0.110 | 1.00 | 0.00 |
| ATOM | 459 | CD2 | TYR | 31 | 5.598 | −10.112 | 1.922 | 1.00 | 0.00 |
| ATOM | 460 | CE1 | TYR | 31 | 6.440 | −11.419 | −0.355 | 1.00 | 0.00 |
| ATOM | 461 | CE2 | TYR | 31 | 5.023 | −11.365 | 1.623 | 1.00 | 0.00 |
| ATOM | 462 | CZ | TYR | 31 | 5.497 | −12.066 | 0.515 | 1.00 | 0.00 |
| ATOM | 463 | OH | TYR | 31 | 4.933 | −13.253 | 0.231 | 1.00 | 0.00 |
| ATOM | 464 | HA | TYR | 31 | 5.409 | −6.893 | 0.010 | 1.00 | 0.00 |
| ATOM | 465 | HB3 | TYR | 31 | 7.069 | −7.845 | 2.388 | 1.00 | 0.00 |
| ATOM | 466 | HB2 | TYR | 31 | 7.729 | −7.816 | 0.806 | 1.00 | 0.00 |
| ATOM | 467 | HD2 | TYR | 31 | 5.179 | −9.621 | 2.788 | 1.00 | 0.00 |
| ATOM | 468 | HE2 | TYR | 31 | 4.159 | −11.754 | 2.139 | 1.00 | 0.00 |
| ATOM | 469 | HE1 | TYR | 31 | 6.727 | −11.929 | −1.263 | 1.00 | 0.00 |
| ATOM | 470 | HD1 | TYR | 31 | 7.605 | −9.660 | −0.793 | 1.00 | 0.00 |
| ATOM | 471 | HH | TYR | 31 | 5.218 | −13.603 | −0.616 | 1.00 | 0.00 |
| ATOM | 472 | H | TYR | 31 | 7.627 | −5.605 | 1.202 | 1.00 | 0.00 |
| ATOM | 473 | N | VAL | 32 | 3.618 | −7.552 | 1.784 | 1.00 | 0.00 |
| ATOM | 474 | CA | VAL | 32 | 2.357 | −7.409 | 2.561 | 1.00 | 0.00 |
| ATOM | 475 | C | VAL | 32 | 2.086 | −8.617 | 3.475 | 1.00 | 0.00 |
| ATOM | 476 | O | VAL | 32 | 2.548 | −9.704 | 3.150 | 1.00 | 0.00 |
| ATOM | 477 | CB | VAL | 32 | 1.106 | −7.177 | 1.667 | 1.00 | 0.00 |
| ATOM | 478 | CG1 | VAL | 32 | −0.182 | −6.772 | 2.407 | 1.00 | 0.00 |
| ATOM | 479 | CG2 | VAL | 32 | 1.475 | −5.883 | 0.858 | 1.00 | 0.00 |
| ATOM | 480 | HA | VAL | 32 | 2.429 | −6.536 | 3.208 | 1.00 | 0.00 |
| ATOM | 481 | HB | VAL | 32 | 0.941 | −8.053 | 1.040 | 1.00 | 0.00 |
| ATOM | 482 | HG11 | VAL | 32 | 0.066 | −6.104 | 3.232 | 1.00 | 0.00 |
| ATOM | 483 | HG12 | VAL | 32 | −0.910 | −6.282 | 1.760 | 1.00 | 0.00 |
| ATOM | 484 | HG13 | VAL | 32 | −0.638 | −7.636 | 2.889 | 1.00 | 0.00 |
| ATOM | 485 | HG21 | VAL | 32 | 1.697 | −4.993 | 1.446 | 1.00 | 0.00 |
| ATOM | 486 | HG22 | VAL | 32 | 2.278 | −6.059 | 0.142 | 1.00 | 0.00 |
| ATOM | 487 | HG23 | VAL | 32 | 0.635 | −5.626 | 0.213 | 1.00 | 0.00 |
| ATOM | 488 | H | VAL | 32 | 3.636 | −8.207 | 1.015 | 1.00 | 0.00 |
| ATOM | 489 | N | LYS | 33 | 1.374 | −8.389 | 4.630 | 1.00 | 0.00 |
| ATOM | 490 | CA | LYS | 33 | 0.841 | −9.414 | 5.529 | 1.00 | 0.00 |
| ATOM | 491 | C | LYS | 33 | −0.626 | −9.818 | 5.338 | 1.00 | 0.00 |
| ATOM | 492 | O | LYS | 33 | −0.846 | −11.032 | 5.077 | 1.00 | 0.00 |
| ATOM | 493 | CB | LYS | 33 | 1.178 | −9.058 | 7.040 | 1.00 | 0.00 |
| ATOM | 494 | CG | LYS | 33 | 0.803 | −10.132 | 8.006 | 1.00 | 0.00 |
| ATOM | 495 | CD | LYS | 33 | 1.319 | −11.519 | 7.695 | 1.00 | 0.00 |
| ATOM | 496 | CE | LYS | 33 | 2.836 | −11.526 | 7.387 | 1.00 | 0.00 |
| ATOM | 497 | NZ | LYS | 33 | 3.386 | −12.887 | 7.264 | 1.00 | 0.00 |
| ATOM | 498 | HA | LYS | 33 | 1.362 | −10.353 | 5.338 | 1.00 | 0.00 |
| ATOM | 499 | HB3 | LYS | 33 | 0.726 | −8.134 | 7.400 | 1.00 | 0.00 |
| ATOM | 500 | HB2 | LYS | 33 | 2.253 | −8.877 | 7.050 | 1.00 | 0.00 |
| ATOM | 501 | HG3 | LYS | 33 | −0.274 | −10.111 | 8.171 | 1.00 | 0.00 |
| ATOM | 502 | HG2 | LYS | 33 | 1.236 | −9.763 | 8.935 | 1.00 | 0.00 |
| ATOM | 503 | HD3 | LYS | 33 | 0.769 | −11.940 | 6.853 | 1.00 | 0.00 |
| ATOM | 504 | HD2 | LYS | 33 | 1.063 | −12.158 | 8.540 | 1.00 | 0.00 |
| ATOM | 505 | HE3 | LYS | 33 | 3.377 | −10.910 | 8.105 | 1.00 | 0.00 |
| ATOM | 506 | HE2 | LYS | 33 | 3.114 | −11.053 | 6.445 | 1.00 | 0.00 |
| ATOM | 507 | HZ1 | LYS | 33 | 4.395 | −12.864 | 7.248 | 1.00 | 0.00 |
| ATOM | 508 | HZ2 | LYS | 33 | 3.119 | −13.413 | 8.084 | 1.00 | 0.00 |
| ATOM | 509 | HZ3 | LYS | 33 | 3.080 | −13.285 | 6.388 | 1.00 | 0.00 |
| ATOM | 510 | H | LYS | 33 | 1.131 | −7.434 | 4.856 | 1.00 | 0.00 |
| ATOM | 511 | N | SER | 34 | −1.561 | −8.858 | 5.388 | 1.00 | 0.00 |
| ATOM | 512 | CA | SER | 34 | −2.993 | −8.925 | 5.145 | 1.00 | 0.00 |
| ATOM | 513 | C | SER | 34 | −3.656 | −7.530 | 5.108 | 1.00 | 0.00 |
| ATOM | 514 | O | SER | 34 | −3.121 | −6.482 | 5.539 | 1.00 | 0.00 |
| ATOM | 515 | CB | SER | 34 | −3.761 | −9.832 | 6.047 | 1.00 | 0.00 |
| ATOM | 516 | OG | SER | 34 | −3.798 | −9.290 | 7.356 | 1.00 | 0.00 |
| ATOM | 517 | HA | SER | 34 | −3.084 | −9.320 | 4.133 | 1.00 | 0.00 |
| ATOM | 518 | HB3 | SER | 34 | −3.319 | −10.818 | 6.189 | 1.00 | 0.00 |
| ATOM | 519 | HB2 | SER | 34 | −4.789 | −9.902 | 5.690 | 1.00 | 0.00 |
| ATOM | 520 | HG | SER | 34 | −4.574 | −8.765 | 7.568 | 1.00 | 0.00 |
| ATOM | 521 | H | SER | 34 | −1.183 | −7.935 | 5.547 | 1.00 | 0.00 |
| ATOM | 522 | N | VAL | 35 | −4.927 | −7.439 | 4.756 | 1.00 | 0.00 |
| ATOM | 523 | CA | VAL | 35 | −5.831 | −6.367 | 5.196 | 1.00 | 0.00 |
| ATOM | 524 | C | VAL | 35 | −6.309 | −6.512 | 6.681 | 1.00 | 0.00 |
| ATOM | 525 | O | VAL | 35 | −6.174 | −7.622 | 7.168 | 1.00 | 0.00 |
| ATOM | 526 | CB | VAL | 35 | −7.155 | −6.303 | 4.472 | 1.00 | 0.00 |
| ATOM | 527 | CG1 | VAL | 35 | −6.979 | −6.078 | 2.955 | 1.00 | 0.00 |
| ATOM | 528 | CG2 | VAL | 35 | −7.853 | −7.661 | 4.575 | 1.00 | 0.00 |
| ATOM | 529 | HA | VAL | 35 | −5.300 | −5.433 | 5.014 | 1.00 | 0.00 |
| ATOM | 530 | HB | VAL | 35 | −7.844 | −5.545 | 4.843 | 1.00 | 0.00 |
| ATOM | 531 | HG11 | VAL | 35 | −7.958 | −5.783 | 2.580 | 1.00 | 0.00 |
| ATOM | 532 | HG12 | VAL | 35 | −6.490 | −5.133 | 2.713 | 1.00 | 0.00 |
| ATOM | 533 | HG13 | VAL | 35 | −6.567 | −6.970 | 2.484 | 1.00 | 0.00 |
| ATOM | 534 | HG21 | VAL | 35 | −8.812 | −7.558 | 4.065 | 1.00 | 0.00 |
| ATOM | 535 | HG22 | VAL | 35 | −7.255 | −8.445 | 4.110 | 1.00 | 0.00 |
| ATOM | 536 | HG23 | VAL | 35 | −8.098 | −7.897 | 5.611 | 1.00 | 0.00 |
| ATOM | 537 | H | VAL | 35 | −5.317 | −8.306 | 4.413 | 1.00 | 0.00 |
| ATOM | 538 | N | VAL | 36 | −6.899 | −5.381 | 7.121 | 1.00 | 0.00 |
| ATOM | 539 | CA | VAL | 36 | −7.385 | −5.197 | 8.494 | 1.00 | 0.00 |
| ATOM | 540 | C | VAL | 36 | −8.957 | −5.231 | 8.726 | 1.00 | 0.00 |
| ATOM | 541 | O | VAL | 36 | −9.374 | −5.125 | 9.860 | 1.00 | 0.00 |
| ATOM | 542 | CB | VAL | 36 | −6.816 | −3.783 | 8.988 | 1.00 | 0.00 |
| ATOM | 543 | CG1 | VAL | 36 | −7.261 | −3.134 | 10.290 | 1.00 | 0.00 |
| ATOM | 544 | CG2 | VAL | 36 | −5.288 | −4.012 | 8.966 | 1.00 | 0.00 |
| ATOM | 545 | HA | VAL | 36 | −6.852 | −5.934 | 9.095 | 1.00 | 0.00 |
| ATOM | 546 | HB | VAL | 36 | −7.081 | −3.018 | 8.259 | 1.00 | 0.00 |
| ATOM | 547 | HG11 | VAL | 36 | −6.829 | −3.710 | 11.108 | 1.00 | 0.00 |
| ATOM | 548 | HG12 | VAL | 36 | −6.839 | −2.130 | 10.270 | 1.00 | 0.00 |
| ATOM | 549 | HG13 | VAL | 36 | −8.336 | −2.976 | 10.389 | 1.00 | 0.00 |
| ATOM | 550 | HG21 | VAL | 36 | −5.148 | −4.967 | 9.471 | 1.00 | 0.00 |
| ATOM | 551 | HG22 | VAL | 36 | −4.846 | −3.989 | 7.971 | 1.00 | 0.00 |
| ATOM | 552 | HG23 | VAL | 36 | −4.737 | −3.260 | 9.532 | 1.00 | 0.00 |
| ATOM | 553 | H | VAL | 36 | −6.864 | −4.561 | 6.532 | 1.00 | 0.00 |
| ATOM | 554 | N | LYS | 37 | −9.746 | −5.490 | 7.679 | 1.00 | 0.00 |
| ATOM | 555 | CA | LYS | 37 | −11.268 | −5.276 | 7.697 | 1.00 | 0.00 |
| ATOM | 556 | C | LYS | 37 | −11.557 | −3.917 | 8.351 | 1.00 | 0.00 |
| ATOM | 557 | O | LYS | 37 | −12.295 | −3.788 | 9.321 | 1.00 | 0.00 |
| ATOM | 558 | CB | LYS | 37 | −11.920 | −6.524 | 8.358 | 1.00 | 0.00 |
| ATOM | 559 | CG | LYS | 37 | −11.414 | −7.915 | 7.903 | 1.00 | 0.00 |
| ATOM | 560 | CD | LYS | 37 | −11.850 | −8.219 | 6.419 | 1.00 | 0.00 |
| ATOM | 561 | CE | LYS | 37 | −13.357 | −8.178 | 6.197 | 1.00 | 0.00 |
| ATOM | 562 | NZ | LYS | 37 | −13.795 | −8.614 | 4.877 | 1.00 | 0.00 |
| ATOM | 563 | HA | LYS | 37 | −11.586 | −5.162 | 6.662 | 1.00 | 0.00 |
| ATOM | 564 | HB3 | LYS | 37 | −12.991 | −6.408 | 8.195 | 1.00 | 0.00 |
| ATOM | 565 | HB2 | LYS | 37 | −11.631 | −6.443 | 9.407 | 1.00 | 0.00 |
| ATOM | 566 | HG3 | LYS | 37 | −11.979 | −8.650 | 8.476 | 1.00 | 0.00 |
| ATOM | 567 | HG2 | LYS | 37 | −10.338 | −8.022 | 8.041 | 1.00 | 0.00 |
| ATOM | 568 | HD3 | LYS | 37 | −11.542 | −9.251 | 6.244 | 1.00 | 0.00 |
| ATOM | 569 | HD2 | LYS | 37 | −11.390 | −7.486 | 5.758 | 1.00 | 0.00 |
| ATOM | 570 | HE3 | LYS | 37 | −13.672 | −7.145 | 6.336 | 1.00 | 0.00 |
| ATOM | 571 | HE2 | LYS | 37 | −13.829 | −8.764 | 6.987 | 1.00 | 0.00 |
| ATOM | 572 | HZ1 | LYS | 37 | −13.336 | −9.458 | 4.564 | 1.00 | 0.00 |
| ATOM | 573 | HZ2 | LYS | 37 | −14.799 | −8.643 | 4.781 | 1.00 | 0.00 |
| ATOM | 574 | HZ3 | LYS | 37 | −13.500 | −7.913 | 4.211 | 1.00 | 0.00 |
| ATOM | 575 | H | LYS | 37 | −9.357 | −5.840 | 6.815 | 1.00 | 0.00 |
| ATOM | 576 | N | GLY | 38 | −10.786 | −2.884 | 7.914 | 1.00 | 0.00 |
| ATOM | 577 | CA | GLY | 38 | −10.655 | −1.622 | 8.674 | 1.00 | 0.00 |
| ATOM | 578 | C | GLY | 38 | −10.365 | −0.384 | 7.898 | 1.00 | 0.00 |
| ATOM | 579 | O | GLY | 38 | −10.066 | 0.714 | 8.395 | 1.00 | 0.00 |
| ATOM | 580 | HA3 | GLY | 38 | −9.845 | −1.679 | 9.402 | 1.00 | 0.00 |
| ATOM | 581 | HA2 | GLY | 38 | −11.569 | −1.353 | 9.204 | 1.00 | 0.00 |
| ATOM | 582 | H | GLY | 38 | −10.204 | −3.020 | 7.100 | 1.00 | 0.00 |
| ATOM | 583 | N | GLY | 39 | −10.448 | −0.521 | 6.545 | 1.00 | 0.00 |
| ATOM | 584 | CA | GLY | 39 | −10.103 | 0.532 | 5.617 | 1.00 | 0.00 |
| ATOM | 585 | C | GLY | 39 | −10.437 | 0.180 | 4.192 | 1.00 | 0.00 |
| ATOM | 586 | O | GLY | 39 | −10.785 | −0.959 | 3.904 | 1.00 | 0.00 |
| ATOM | 587 | HA3 | GLY | 39 | −9.051 | 0.815 | 5.659 | 1.00 | 0.00 |
| ATOM | 588 | HA2 | GLY | 39 | −10.675 | 1.430 | 5.847 | 1.00 | 0.00 |
| ATOM | 589 | H | GLY | 39 | −10.689 | −1.432 | 6.180 | 1.00 | 0.00 |
| ATOM | 590 | N | ALA | 40 | −10.150 | 1.093 | 3.270 | 1.00 | 0.00 |
| ATOM | 591 | CA | ALA | 40 | −10.505 | 1.070 | 1.831 | 1.00 | 0.00 |
| ATOM | 592 | C | ALA | 40 | −10.120 | −0.237 | 1.080 | 1.00 | 0.00 |
| ATOM | 593 | O | ALA | 40 | −10.779 | −0.639 | 0.109 | 1.00 | 0.00 |
| ATOM | 594 | CB | ALA | 40 | −10.021 | 2.343 | 1.138 | 1.00 | 0.00 |
| ATOM | 595 | HA | ALA | 40 | −11.594 | 1.013 | 1.840 | 1.00 | 0.00 |
| ATOM | 596 | HB1 | ALA | 40 | −10.383 | 3.252 | 1.620 | 1.00 | 0.00 |
| ATOM | 597 | HB2 | ALA | 40 | −8.949 | 2.501 | 1.019 | 1.00 | 0.00 |
| ATOM | 598 | HB3 | ALA | 40 | −10.468 | 2.283 | 0.147 | 1.00 | 0.00 |
| ATOM | 599 | H | ALA | 40 | −9.831 | 1.974 | 3.650 | 1.00 | 0.00 |
| ATOM | 600 | N | ALA | 41 | −9.000 | −0.872 | 1.466 | 1.00 | 0.00 |
| ATOM | 601 | CA | ALA | 41 | −8.478 | −2.023 | 0.791 | 1.00 | 0.00 |
| ATOM | 602 | C | ALA | 41 | −9.433 | −3.235 | 0.873 | 1.00 | 0.00 |
| ATOM | 603 | O | ALA | 41 | −9.556 | −3.995 | −0.043 | 1.00 | 0.00 |
| ATOM | 604 | CB | ALA | 41 | −7.129 | −2.410 | 1.364 | 1.00 | 0.00 |
| ATOM | 605 | HA | ALA | 41 | −8.343 | −1.812 | −0.270 | 1.00 | 0.00 |
| ATOM | 606 | HB1 | ALA | 41 | −6.482 | −1.536 | 1.431 | 1.00 | 0.00 |
| ATOM | 607 | HB2 | ALA | 41 | −7.289 | −2.823 | 2.360 | 1.00 | 0.00 |
| ATOM | 608 | HB3 | ALA | 41 | −6.628 | −3.173 | 0.768 | 1.00 | 0.00 |
| ATOM | 609 | H | ALA | 41 | −8.493 | −0.401 | 2.201 | 1.00 | 0.00 |
| ATOM | 610 | N | ASP | 42 | −10.068 | −3.414 | 2.100 | 1.00 | 0.00 |
| ATOM | 611 | CA | ASP | 42 | −10.897 | −4.555 | 2.218 | 1.00 | 0.00 |
| ATOM | 612 | C | ASP | 42 | −12.239 | −4.168 | 1.647 | 1.00 | 0.00 |
| ATOM | 613 | O | ASP | 42 | −12.703 | −4.878 | 0.770 | 1.00 | 0.00 |
| ATOM | 614 | CB | ASP | 42 | −11.078 | −4.981 | 3.682 | 1.00 | 0.00 |
| ATOM | 615 | CG | ASP | 42 | −12.150 | −6.095 | 3.672 | 1.00 | 0.00 |
| ATOM | 616 | OD1 | ASP | 42 | −11.933 | −7.202 | 3.236 | 1.00 | 0.00 |
| ATOM | 617 | OD2 | ASP | 42 | −13.269 | −5.789 | 4.192 | 1.00 | 0.00 |
| ATOM | 618 | HA | ASP | 42 | −10.555 | −5.363 | 1.571 | 1.00 | 0.00 |
| ATOM | 619 | HB3 | ASP | 42 | −11.418 | −4.132 | 4.275 | 1.00 | 0.00 |
| ATOM | 620 | HB2 | ASP | 42 | −10.176 | −5.456 | 4.067 | 1.00 | 0.00 |
| ATOM | 621 | H | ASP | 42 | −10.009 | −2.747 | 2.856 | 1.00 | 0.00 |
| ATOM | 622 | N | VAL | 43 | −12.866 | −3.082 | 2.073 | 1.00 | 0.00 |
| ATOM | 623 | CA | VAL | 43 | −14.237 | −2.660 | 1.745 | 1.00 | 0.00 |
| ATOM | 624 | C | VAL | 43 | −14.334 | −2.388 | 0.248 | 1.00 | 0.00 |
| ATOM | 625 | O | VAL | 43 | −15.526 | −2.409 | −0.256 | 1.00 | 0.00 |
| ATOM | 626 | CB | VAL | 43 | −14.671 | −1.419 | 2.527 | 1.00 | 0.00 |
| ATOM | 627 | CG1 | VAL | 43 | −13.802 | −0.232 | 2.273 | 1.00 | 0.00 |
| ATOM | 628 | CG2 | VAL | 43 | −16.142 | −1.089 | 2.501 | 1.00 | 0.00 |
| ATOM | 629 | HA | VAL | 43 | −14.918 | −3.480 | 1.975 | 1.00 | 0.00 |
| ATOM | 630 | HB | VAL | 43 | −14.410 | −1.707 | 3.545 | 1.00 | 0.00 |
| ATOM | 631 | HG11 | VAL | 43 | −12.896 | −0.329 | 2.873 | 1.00 | 0.00 |
| ATOM | 632 | HG12 | VAL | 43 | −13.595 | −0.088 | 1.213 | 1.00 | 0.00 |
| ATOM | 633 | HG13 | VAL | 43 | −14.212 | 0.666 | 2.736 | 1.00 | 0.00 |
| ATOM | 634 | HG21 | VAL | 43 | −16.473 | −1.119 | 1.463 | 1.00 | 0.00 |
| ATOM | 635 | HG22 | VAL | 43 | −16.766 | −1.746 | 3.107 | 1.00 | 0.00 |
| ATOM | 636 | HG23 | VAL | 43 | −16.284 | −0.067 | 2.853 | 1.00 | 0.00 |
| ATOM | 637 | H | VAL | 43 | −12.346 | −2.728 | 2.864 | 1.00 | 0.00 |
| ATOM | 638 | N | ASP | 44 | −13.281 | −2.048 | −0.459 | 1.00 | 0.00 |
| ATOM | 639 | CA | ASP | 44 | −13.419 | −1.664 | −1.923 | 1.00 | 0.00 |
| ATOM | 640 | C | ASP | 44 | −12.452 | −2.476 | −2.824 | 1.00 | 0.00 |
| ATOM | 641 | O | ASP | 44 | −12.554 | −2.404 | −4.033 | 1.00 | 0.00 |
| ATOM | 642 | CB | ASP | 44 | −13.129 | −0.167 | −1.974 | 1.00 | 0.00 |
| ATOM | 643 | CG | ASP | 44 | −13.476 | 0.397 | −3.392 | 1.00 | 0.00 |
| ATOM | 644 | OD1 | ASP | 44 | −14.636 | 0.092 | −3.784 | 1.00 | 0.00 |
| ATOM | 645 | OD2 | ASP | 44 | −12.673 | 1.128 | −3.987 | 1.00 | 0.00 |
| ATOM | 646 | HA | ASP | 44 | −14.446 | −1.937 | −2.170 | 1.00 | 0.00 |
| ATOM | 647 | HB3 | ASP | 44 | −12.087 | 0.125 | −1.835 | 1.00 | 0.00 |
| ATOM | 648 | HB2 | ASP | 44 | −13.664 | 0.299 | −1.146 | 1.00 | 0.00 |
| ATOM | 649 | H | ASP | 44 | −12.369 | −1.960 | −0.036 | 1.00 | 0.00 |
| ATOM | 650 | N | GLY | 45 | −11.487 | −3.254 | −2.200 | 1.00 | 0.00 |
| ATOM | 651 | CA | GLY | 45 | −10.600 | −4.113 | −2.949 | 1.00 | 0.00 |
| ATOM | 652 | C | GLY | 45 | −10.883 | −5.636 | −2.791 | 1.00 | 0.00 |
| ATOM | 653 | O | GLY | 45 | −10.566 | −6.400 | −3.757 | 1.00 | 0.00 |
| ATOM | 654 | HA3 | GLY | 45 | −9.619 | −3.967 | −2.496 | 1.00 | 0.00 |
| ATOM | 655 | HA2 | GLY | 45 | −10.627 | −3.801 | −3.992 | 1.00 | 0.00 |
| ATOM | 656 | H | GLY | 45 | −11.357 | −3.273 | −1.199 | 1.00 | 0.00 |
| ATOM | 657 | N | ARG | 46 | −11.476 | −6.097 | −1.641 | 1.00 | 0.00 |
| ATOM | 658 | CA | ARG | 46 | −11.916 | −7.465 | −1.351 | 1.00 | 0.00 |
| ATOM | 659 | C | ARG | 46 | −10.968 | −8.598 | −1.760 | 1.00 | 0.00 |
| ATOM | 660 | O | ARG | 46 | −11.300 | −9.688 | −2.286 | 1.00 | 0.00 |
| ATOM | 661 | CB | ARG | 46 | −13.365 | −7.744 | −1.813 | 1.00 | 0.00 |
| ATOM | 662 | CG | ARG | 46 | −14.381 | −6.731 | −1.296 | 1.00 | 0.00 |
| ATOM | 663 | CD | ARG | 46 | −15.789 | −6.953 | −1.720 | 1.00 | 0.00 |
| ATOM | 664 | NE | ARG | 46 | −15.873 | −6.544 | −3.064 | 1.00 | 0.00 |
| ATOM | 665 | CZ | ARG | 46 | −16.193 | −7.219 | −4.149 | 1.00 | 0.00 |
| ATOM | 666 | NH1 | ARG | 46 | −15.952 | −6.776 | −5.313 | 1.00 | 0.00 |
| ATOM | 667 | NH2 | ARG | 46 | −16.662 | −8.426 | −4.124 | 1.00 | 0.00 |
| ATOM | 668 | HA | ARG | 46 | −11.977 | −7.591 | −0.270 | 1.00 | 0.00 |
| ATOM | 669 | HB3 | ARG | 46 | −13.553 | −8.788 | −1.562 | 1.00 | 0.00 |
| ATOM | 670 | HB2 | ARG | 46 | −13.329 | −7.728 | −2.902 | 1.00 | 0.00 |
| ATOM | 671 | HG3 | ARG | 46 | −14.173 | −5.686 | −1.526 | 1.00 | 0.00 |
| ATOM | 672 | HG2 | ARG | 46 | −14.373 | −6.907 | −0.220 | 1.00 | 0.00 |
| ATOM | 673 | HD3 | ARG | 46 | −16.508 | −6.477 | −1.054 | 1.00 | 0.00 |
| ATOM | 674 | HD2 | ARG | 46 | −15.943 | −8.015 | −1.524 | 1.00 | 0.00 |
| ATOM | 675 | HE | ARG | 46 | −15.838 | −5.550 | −3.241 | 1.00 | 0.00 |
| ATOM | 676 | HH12 | ARG | 46 | −16.023 | −7.334 | −6.152 | 1.00 | 0.00 |
| ATOM | 677 | HH11 | ARG | 46 | −15.520 | −5.866 | −5.379 | 1.00 | 0.00 |
| ATOM | 678 | HH22 | ARG | 46 | −16.647 | −9.049 | −4.919 | 1.00 | 0.00 |
| ATOM | 679 | HH21 | ARG | 46 | −16.826 | −8.818 | −3.208 | 1.00 | 0.00 |
| ATOM | 680 | H | ARG | 46 | −11.687 | −5.440 | −0.903 | 1.00 | 0.00 |
| ATOM | 681 | N | LEU | 47 | −9.650 | −8.384 | −1.510 | 1.00 | 0.00 |
| ATOM | 682 | CA | LEU | 47 | −8.429 | −9.025 | −2.006 | 1.00 | 0.00 |
| ATOM | 683 | C | LEU | 47 | −7.580 | −9.887 | −1.060 | 1.00 | 0.00 |
| ATOM | 684 | O | LEU | 47 | −6.598 | −10.514 | −1.499 | 1.00 | 0.00 |
| ATOM | 685 | CB | LEU | 47 | −7.667 | −7.903 | −2.634 | 1.00 | 0.00 |
| ATOM | 686 | CG | LEU | 47 | −7.504 | −6.584 | −1.853 | 1.00 | 0.00 |
| ATOM | 687 | CD1 | LEU | 47 | −6.565 | −6.657 | −0.685 | 1.00 | 0.00 |
| ATOM | 688 | CD2 | LEU | 47 | −7.214 | −5.398 | −2.681 | 1.00 | 0.00 |
| ATOM | 689 | HA | LEU | 47 | −8.703 | −9.667 | −2.843 | 1.00 | 0.00 |
| ATOM | 690 | HB3 | LEU | 47 | −8.208 | −7.823 | −3.577 | 1.00 | 0.00 |
| ATOM | 691 | HB2 | LEU | 47 | −6.668 | −8.291 | −2.836 | 1.00 | 0.00 |
| ATOM | 692 | HG | LEU | 47 | −8.437 | −6.325 | −1.353 | 1.00 | 0.00 |
| ATOM | 693 | HD21 | LEU | 47 | −7.055 | −4.519 | −2.056 | 1.00 | 0.00 |
| ATOM | 694 | HD22 | LEU | 47 | −6.296 | −5.500 | −3.259 | 1.00 | 0.00 |
| ATOM | 695 | HD23 | LEU | 47 | −8.068 | −5.202 | −3.329 | 1.00 | 0.00 |
| ATOM | 696 | HD11 | LEU | 47 | −5.541 | −6.740 | −1.049 | 1.00 | 0.00 |
| ATOM | 697 | HD12 | LEU | 47 | −6.620 | −5.717 | −0.136 | 1.00 | 0.00 |
| ATOM | 698 | HD13 | LEU | 47 | −6.816 | −7.407 | 0.066 | 1.00 | 0.00 |
| ATOM | 699 | H | LEU | 47 | −9.513 | −7.481 | −1.078 | 1.00 | 0.00 |
| ATOM | 700 | N | ALA | 48 | −7.883 | −9.966 | 0.285 | 1.00 | 0.00 |
| ATOM | 701 | CA | ALA | 48 | −7.057 | −10.487 | 1.380 | 1.00 | 0.00 |
| ATOM | 702 | C | ALA | 48 | −5.586 | −9.925 | 1.600 | 1.00 | 0.00 |
| ATOM | 703 | O | ALA | 48 | −5.261 | −9.699 | 2.753 | 1.00 | 0.00 |
| ATOM | 704 | CB | ALA | 48 | −7.184 | −12.014 | 1.404 | 1.00 | 0.00 |
| ATOM | 705 | HA | ALA | 48 | −7.601 | −10.137 | 2.257 | 1.00 | 0.00 |
| ATOM | 706 | HB1 | ALA | 48 | −6.583 | −12.503 | 0.638 | 1.00 | 0.00 |
| ATOM | 707 | HB2 | ALA | 48 | −6.911 | −12.325 | 2.413 | 1.00 | 0.00 |
| ATOM | 708 | HB3 | ALA | 48 | −8.218 | −12.297 | 1.206 | 1.00 | 0.00 |
| ATOM | 709 | H | ALA | 48 | −8.789 | −9.580 | 0.506 | 1.00 | 0.00 |
| ATOM | 710 | N | ALA | 49 | −4.759 | −9.780 | 0.598 | 1.00 | 0.00 |
| ATOM | 711 | CA | ALA | 49 | −3.426 | −9.239 | 0.543 | 1.00 | 0.00 |
| ATOM | 712 | C | ALA | 49 | −2.347 | −10.144 | 1.262 | 1.00 | 0.00 |
| ATOM | 713 | O | ALA | 49 | −2.692 | −11.044 | 2.076 | 1.00 | 0.00 |
| ATOM | 714 | CB | ALA | 49 | −3.374 | −7.717 | 0.792 | 1.00 | 0.00 |
| ATOM | 715 | HA | ALA | 49 | −3.132 | −9.253 | −0.506 | 1.00 | 0.00 |
| ATOM | 716 | HB1 | ALA | 49 | −3.032 | −7.567 | 1.817 | 1.00 | 0.00 |
| ATOM | 717 | HB2 | ALA | 49 | −2.633 | −7.225 | 0.164 | 1.00 | 0.00 |
| ATOM | 718 | HB3 | ALA | 49 | −4.341 | −7.214 | 0.783 | 1.00 | 0.00 |
| ATOM | 719 | H | ALA | 49 | −5.074 | −10.036 | −0.327 | 1.00 | 0.00 |
| ATOM | 720 | N | GLY | 50 | −1.061 | −9.910 | 1.012 | 1.00 | 0.00 |
| ATOM | 721 | CA | GLY | 50 | −0.022 | −10.861 | 1.457 | 1.00 | 0.00 |
| ATOM | 722 | C | GLY | 50 | 1.301 | −10.881 | 0.711 | 1.00 | 0.00 |
| ATOM | 723 | O | GLY | 50 | 2.157 | −11.740 | 0.993 | 1.00 | 0.00 |
| ATOM | 724 | HA3 | GLY | 50 | −0.500 | −11.841 | 1.468 | 1.00 | 0.00 |
| ATOM | 725 | HA2 | GLY | 50 | 0.162 | −10.634 | 2.507 | 1.00 | 0.00 |
| ATOM | 726 | H | GLY | 50 | −0.776 | −9.199 | 0.354 | 1.00 | 0.00 |
| ATOM | 727 | N | ASP | 51 | 1.543 | −10.083 | −0.340 | 1.00 | 0.00 |
| ATOM | 728 | CA | ASP | 51 | 2.718 | −10.186 | −1.189 | 1.00 | 0.00 |
| ATOM | 729 | C | ASP | 51 | 3.369 | −8.866 | −1.586 | 1.00 | 0.00 |
| ATOM | 730 | O | ASP | 51 | 4.583 | −8.821 | −1.580 | 1.00 | 0.00 |
| ATOM | 731 | CB | ASP | 51 | 2.441 | −10.995 | −2.468 | 1.00 | 0.00 |
| ATOM | 732 | CG | ASP | 51 | 3.646 | −11.733 | −3.039 | 1.00 | 0.00 |
| ATOM | 733 | OD1 | ASP | 51 | 3.962 | −12.877 | −2.627 | 1.00 | 0.00 |
| ATOM | 734 | OD2 | ASP | 51 | 4.222 | −11.136 | −3.980 | 1.00 | 0.00 |
| ATOM | 735 | HA | ASP | 51 | 3.560 | −10.665 | −0.689 | 1.00 | 0.00 |
| ATOM | 736 | HB3 | ASP | 51 | 1.970 | −10.274 | −3.136 | 1.00 | 0.00 |
| ATOM | 737 | HB2 | ASP | 51 | 1.679 | −11.761 | −2.325 | 1.00 | 0.00 |
| ATOM | 738 | H | ASP | 51 | 0.827 | −9.396 | −0.526 | 1.00 | 0.00 |
| ATOM | 739 | N | GLN | 52 | 2.609 | −7.784 | −1.904 | 1.00 | 0.00 |
| ATOM | 740 | CA | GLN | 52 | 3.305 | −6.576 | −2.497 | 1.00 | 0.00 |
| ATOM | 741 | C | GLN | 52 | 2.480 | −5.336 | −2.348 | 1.00 | 0.00 |
| ATOM | 742 | O | GLN | 52 | 1.226 | −5.370 | −2.375 | 1.00 | 0.00 |
| ATOM | 743 | CB | GLN | 52 | 3.734 | −6.859 | −3.938 | 1.00 | 0.00 |
| ATOM | 744 | CG | GLN | 52 | 5.187 | −6.325 | −4.321 | 1.00 | 0.00 |
| ATOM | 745 | CD | GLN | 52 | 5.433 | −6.459 | −5.850 | 1.00 | 0.00 |
| ATOM | 746 | OE1 | GLN | 52 | 4.719 | −7.087 | −6.610 | 1.00 | 0.00 |
| ATOM | 747 | NE2 | GLN | 52 | 6.626 | −6.057 | −6.295 | 1.00 | 0.00 |
| ATOM | 748 | HA | GLN | 52 | 4.215 | −6.420 | −1.918 | 1.00 | 0.00 |
| ATOM | 749 | HB3 | GLN | 52 | 3.041 | −6.411 | −4.651 | 1.00 | 0.00 |
| ATOM | 750 | HB2 | GLN | 52 | 3.735 | −7.936 | −4.106 | 1.00 | 0.00 |
| ATOM | 751 | HG3 | GLN | 52 | 5.981 | −6.933 | −3.890 | 1.00 | 0.00 |
| ATOM | 752 | HG2 | GLN | 52 | 5.206 | −5.246 | −4.169 | 1.00 | 0.00 |
| ATOM | 753 | HE22 | GLN | 52 | 7.370 | −5.715 | −5.705 | 1.00 | 0.00 |
| ATOM | 754 | HE21 | GLN | 52 | 6.846 | −6.278 | −7.256 | 1.00 | 0.00 |
| ATOM | 755 | H | GLN | 52 | 1.624 | −7.707 | −1.698 | 1.00 | 0.00 |
| ATOM | 756 | N | LEU | 53 | 3.142 | −4.167 | −2.241 | 1.00 | 0.00 |
| ATOM | 757 | CA | LEU | 53 | 2.605 | −2.802 | −2.613 | 1.00 | 0.00 |
| ATOM | 758 | C | LEU | 53 | 3.511 | −2.208 | −3.612 | 1.00 | 0.00 |
| ATOM | 759 | O | LEU | 53 | 4.748 | −2.176 | −3.500 | 1.00 | 0.00 |
| ATOM | 760 | CB | LEU | 53 | 2.456 | −2.014 | −1.292 | 1.00 | 0.00 |
| ATOM | 761 | CG | LEU | 53 | 1.357 | −0.961 | −1.452 | 1.00 | 0.00 |
| ATOM | 762 | CD1 | LEU | 53 | 0.863 | −0.639 | −0.037 | 1.00 | 0.00 |
| ATOM | 763 | CD2 | LEU | 53 | 1.917 | 0.268 | −2.162 | 1.00 | 0.00 |
| ATOM | 764 | HA | LEU | 53 | 1.587 | −2.982 | −2.959 | 1.00 | 0.00 |
| ATOM | 765 | HB3 | LEU | 53 | 3.411 | −1.507 | −1.162 | 1.00 | 0.00 |
| ATOM | 766 | HB2 | LEU | 53 | 2.234 | −2.652 | −0.435 | 1.00 | 0.00 |
| ATOM | 767 | HG | LEU | 53 | 0.477 | −1.371 | −1.947 | 1.00 | 0.00 |
| ATOM | 768 | HD21 | LEU | 53 | 1.246 | 1.072 | −1.860 | 1.00 | 0.00 |
| ATOM | 769 | HD22 | LEU | 53 | 2.899 | 0.546 | −1.781 | 1.00 | 0.00 |
| ATOM | 770 | HD23 | LEU | 53 | 1.877 | 0.181 | −3.249 | 1.00 | 0.00 |
| ATOM | 771 | HD11 | LEU | 53 | 0.193 | 0.220 | 0.012 | 1.00 | 0.00 |
| ATOM | 772 | HD12 | LEU | 53 | 0.111 | −1.409 | 0.134 | 1.00 | 0.00 |
| ATOM | 773 | HD13 | LEU | 53 | 1.687 | −0.593 | 0.674 | 1.00 | 0.00 |
| ATOM | 774 | H | LEU | 53 | 4.147 | −4.271 | −2.233 | 1.00 | 0.00 |
| ATOM | 775 | N | LEU | 54 | 2.867 | −1.711 | −4.667 | 1.00 | 0.00 |
| ATOM | 776 | CA | LEU | 54 | 3.617 | −1.492 | −5.865 | 1.00 | 0.00 |
| ATOM | 777 | C | LEU | 54 | 3.737 | 0.017 | −6.245 | 1.00 | 0.00 |
| ATOM | 778 | O | LEU | 54 | 4.818 | 0.501 | −6.600 | 1.00 | 0.00 |
| ATOM | 779 | CB | LEU | 54 | 2.966 | −2.247 | −7.008 | 1.00 | 0.00 |
| ATOM | 780 | CG | LEU | 54 | 3.659 | −2.305 | −8.378 | 1.00 | 0.00 |
| ATOM | 781 | CD1 | LEU | 54 | 4.942 | −3.172 | −8.337 | 1.00 | 0.00 |
| ATOM | 782 | CD2 | LEU | 54 | 2.704 | −2.983 | −9.316 | 1.00 | 0.00 |
| ATOM | 783 | HA | LEU | 54 | 4.658 | −1.793 | −5.747 | 1.00 | 0.00 |
| ATOM | 784 | HB3 | LEU | 54 | 1.967 | −1.826 | −7.120 | 1.00 | 0.00 |
| ATOM | 785 | HB2 | LEU | 54 | 2.825 | −3.279 | −6.686 | 1.00 | 0.00 |
| ATOM | 786 | HG | LEU | 54 | 3.972 | −1.323 | −8.735 | 1.00 | 0.00 |
| ATOM | 787 | HD21 | LEU | 54 | 1.789 | −2.400 | −9.424 | 1.00 | 0.00 |
| ATOM | 788 | HD22 | LEU | 54 | 3.171 | −3.006 | −10.302 | 1.00 | 0.00 |
| ATOM | 789 | HD23 | LEU | 54 | 2.361 | −3.978 | −9.036 | 1.00 | 0.00 |
| ATOM | 790 | HD11 | LEU | 54 | 5.620 | −2.713 | −7.618 | 1.00 | 0.00 |
| ATOM | 791 | HD12 | LEU | 54 | 4.711 | −4.129 | −7.871 | 1.00 | 0.00 |
| ATOM | 792 | HD13 | LEU | 54 | 5.425 | −3.276 | −9.309 | 1.00 | 0.00 |
| ATOM | 793 | H | LEU | 54 | 1.857 | −1.708 | −4.648 | 1.00 | 0.00 |
| ATOM | 794 | N | SER | 55 | 2.561 | 0.704 | −6.298 | 1.00 | 0.00 |
| ATOM | 795 | CA | SER | 55 | 2.402 | 2.072 | −6.849 | 1.00 | 0.00 |
| ATOM | 796 | C | SER | 55 | 1.253 | 2.848 | −6.206 | 1.00 | 0.00 |
| ATOM | 797 | O | SER | 55 | 0.125 | 2.400 | −6.040 | 1.00 | 0.00 |
| ATOM | 798 | CB | SER | 55 | 2.130 | 1.935 | −8.320 | 1.00 | 0.00 |
| ATOM | 799 | OG | SER | 55 | 3.120 | 1.269 | −9.021 | 1.00 | 0.00 |
| ATOM | 800 | HA | SER | 55 | 3.385 | 2.542 | −6.809 | 1.00 | 0.00 |
| ATOM | 801 | HB3 | SER | 55 | 2.033 | 2.910 | −8.799 | 1.00 | 0.00 |
| ATOM | 802 | HB2 | SER | 55 | 1.185 | 1.413 | −8.465 | 1.00 | 0.00 |
| ATOM | 803 | HG | SER | 55 | 2.639 | 0.700 | −9.627 | 1.00 | 0.00 |
| ATOM | 804 | H | SER | 55 | 1.739 | 0.119 | −6.257 | 1.00 | 0.00 |
| ATOM | 805 | N | VAL | 56 | 1.456 | 4.171 | −6.011 | 1.00 | 0.00 |
| ATOM | 806 | CA | VAL | 56 | 0.348 | 5.049 | −5.471 | 1.00 | 0.00 |
| ATOM | 807 | C | VAL | 56 | 0.366 | 6.394 | −6.218 | 1.00 | 0.00 |
| ATOM | 808 | O | VAL | 56 | 1.403 | 6.743 | −6.752 | 1.00 | 0.00 |
| ATOM | 809 | CB | VAL | 56 | 0.430 | 5.360 | −3.964 | 1.00 | 0.00 |
| ATOM | 810 | CG1 | VAL | 56 | 0.321 | 4.114 | −3.200 | 1.00 | 0.00 |
| ATOM | 811 | CG2 | VAL | 56 | 1.661 | 6.163 | −3.520 | 1.00 | 0.00 |
| ATOM | 812 | HA | VAL | 56 | −0.625 | 4.593 | −5.650 | 1.00 | 0.00 |
| ATOM | 813 | HB | VAL | 56 | −0.480 | 5.907 | −3.716 | 1.00 | 0.00 |
| ATOM | 814 | HG11 | VAL | 56 | 1.180 | 3.502 | −3.474 | 1.00 | 0.00 |
| ATOM | 815 | HG12 | VAL | 56 | 0.366 | 4.258 | −2.120 | 1.00 | 0.00 |
| ATOM | 816 | HG13 | VAL | 56 | −0.670 | 3.667 | −3.275 | 1.00 | 0.00 |
| ATOM | 817 | HG21 | VAL | 56 | 1.707 | 7.140 | −3.999 | 1.00 | 0.00 |
| ATOM | 818 | HG22 | VAL | 56 | 1.667 | 6.297 | −2.438 | 1.00 | 0.00 |
| ATOM | 819 | HG23 | VAL | 56 | 2.603 | 5.711 | −3.830 | 1.00 | 0.00 |
| ATOM | 820 | H | VAL | 56 | 2.290 | 4.620 | −6.362 | 1.00 | 0.00 |
| ATOM | 821 | N | ASP | 57 | −0.754 | 7.109 | −6.239 | 1.00 | 0.00 |
| ATOM | 822 | CA | ASP | 57 | −0.873 | 8.401 | −6.985 | 1.00 | 0.00 |
| ATOM | 823 | C | ASP | 57 | −0.402 | 8.240 | −8.422 | 1.00 | 0.00 |
| ATOM | 824 | O | ASP | 57 | 0.352 | 9.040 | −8.932 | 1.00 | 0.00 |
| ATOM | 825 | CB | ASP | 57 | −0.176 | 9.476 | −6.120 | 1.00 | 0.00 |
| ATOM | 826 | CG | ASP | 57 | −0.265 | 10.905 | −6.679 | 1.00 | 0.00 |
| ATOM | 827 | OD1 | ASP | 57 | −1.145 | 11.278 | −7.543 | 1.00 | 0.00 |
| ATOM | 828 | OD2 | ASP | 57 | 0.654 | 11.724 | −6.413 | 1.00 | 0.00 |
| ATOM | 829 | HA | ASP | 57 | −1.920 | 8.701 | −7.024 | 1.00 | 0.00 |
| ATOM | 830 | HB3 | ASP | 57 | 0.881 | 9.219 | −6.177 | 1.00 | 0.00 |
| ATOM | 831 | HB2 | ASP | 57 | −0.388 | 9.382 | −5.054 | 1.00 | 0.00 |
| ATOM | 832 | H | ASP | 57 | −1.673 | 6.754 | −6.014 | 1.00 | 0.00 |
| ATOM | 833 | N | GLY | 58 | −0.762 | 7.123 | −9.058 | 1.00 | 0.00 |
| ATOM | 834 | CA | GLY | 58 | −0.351 | 6.755 | −10.376 | 1.00 | 0.00 |
| ATOM | 835 | C | GLY | 58 | 1.161 | 6.441 | −10.618 | 1.00 | 0.00 |
| ATOM | 836 | O | GLY | 58 | 1.460 | 6.138 | −11.788 | 1.00 | 0.00 |
| ATOM | 837 | HA3 | GLY | 58 | −0.668 | 7.403 | −11.194 | 1.00 | 0.00 |
| ATOM | 838 | HA2 | GLY | 58 | −0.928 | 5.849 | −10.568 | 1.00 | 0.00 |
| ATOM | 839 | H | GLY | 58 | −1.259 | 6.425 | −8.524 | 1.00 | 0.00 |
| ATOM | 840 | N | ARG | 59 | 2.061 | 6.547 | −9.640 | 1.00 | 0.00 |
| ATOM | 841 | CA | ARG | 59 | 3.493 | 6.324 | −9.836 | 1.00 | 0.00 |
| ATOM | 842 | C | ARG | 59 | 4.089 | 5.377 | −8.834 | 1.00 | 0.00 |
| ATOM | 843 | O | ARG | 59 | 3.575 | 5.232 | −7.710 | 1.00 | 0.00 |
| ATOM | 844 | CB | ARG | 59 | 4.319 | 7.587 | −9.847 | 1.00 | 0.00 |
| ATOM | 845 | CG | ARG | 59 | 3.878 | 8.510 | −10.970 | 1.00 | 0.00 |
| ATOM | 846 | CD | ARG | 59 | 4.675 | 9.885 | −10.944 | 1.00 | 0.00 |
| ATOM | 847 | NE | ARG | 59 | 4.460 | 10.684 | −9.646 | 1.00 | 0.00 |
| ATOM | 848 | CZ | ARG | 59 | 3.293 | 11.025 | −9.161 | 1.00 | 0.00 |
| ATOM | 849 | NH1 | ARG | 59 | 3.143 | 11.430 | −7.964 | 1.00 | 0.00 |
| ATOM | 850 | NH2 | ARG | 59 | 2.227 | 11.056 | −9.900 | 1.00 | 0.00 |
| ATOM | 851 | HA | ARG | 59 | 3.612 | 5.897 | −10.832 | 1.00 | 0.00 |
| ATOM | 852 | HB3 | ARG | 59 | 5.370 | 7.369 | −10.036 | 1.00 | 0.00 |
| ATOM | 853 | HB2 | ARG | 59 | 4.076 | 8.169 | −8.958 | 1.00 | 0.00 |
| ATOM | 854 | HG3 | ARG | 59 | 2.813 | 8.659 | −10.789 | 1.00 | 0.00 |
| ATOM | 855 | HG2 | ARG | 59 | 4.041 | 7.978 | −11.907 | 1.00 | 0.00 |
| ATOM | 856 | HD3 | ARG | 59 | 4.237 | 10.474 | −11.749 | 1.00 | 0.00 |
| ATOM | 857 | HD2 | ARG | 59 | 5.739 | 9.765 | −11.152 | 1.00 | 0.00 |
| ATOM | 858 | HE | ARG | 59 | 5.238 | 10.690 | −9.003 | 1.00 | 0.00 |
| ATOM | 859 | HH12 | ARG | 59 | 2.214 | 11.584 | −7.599 | 1.00 | 0.00 |
| ATOM | 860 | HH11 | ARG | 59 | 3.935 | 11.137 | −7.410 | 1.00 | 0.00 |
| ATOM | 861 | HH22 | ARG | 59 | 1.341 | 11.410 | −9.567 | 1.00 | 0.00 |
| ATOM | 862 | HH21 | ARG | 59 | 2.312 | 10.719 | −10.848 | 1.00 | 0.00 |
| ATOM | 863 | H | ARG | 59 | 1.808 | 6.606 | −8.663 | 1.00 | 0.00 |
| ATOM | 864 | N | SER | 60 | 5.272 | 4.786 | −9.128 | 1.00 | 0.00 |
| ATOM | 865 | CA | SER | 60 | 5.827 | 3.628 | −8.470 | 1.00 | 0.00 |
| ATOM | 866 | C | SER | 60 | 6.203 | 3.942 | −7.060 | 1.00 | 0.00 |
| ATOM | 867 | O | SER | 60 | 6.885 | 4.949 | −6.809 | 1.00 | 0.00 |
| ATOM | 868 | CB | SER | 60 | 7.063 | 3.190 | −9.285 | 1.00 | 0.00 |
| ATOM | 869 | OG | SER | 60 | 7.649 | 2.036 | −8.702 | 1.00 | 0.00 |
| ATOM | 870 | HA | SER | 60 | 5.099 | 2.818 | −8.497 | 1.00 | 0.00 |
| ATOM | 871 | HB3 | SER | 60 | 7.842 | 3.944 | −9.390 | 1.00 | 0.00 |
| ATOM | 872 | HB2 | SER | 60 | 6.699 | 2.917 | −10.275 | 1.00 | 0.00 |
| ATOM | 873 | HG | SER | 60 | 7.185 | 1.254 | −9.009 | 1.00 | 0.00 |
| ATOM | 874 | H | SER | 60 | 5.690 | 4.981 | −10.027 | 1.00 | 0.00 |
| ATOM | 875 | N | LEU | 61 | 5.989 | 3.026 | −6.108 | 1.00 | 0.00 |
| ATOM | 876 | CA | LEU | 61 | 6.529 | 3.160 | −4.729 | 1.00 | 0.00 |
| ATOM | 877 | C | LEU | 61 | 7.487 | 2.026 | −4.220 | 1.00 | 0.00 |
| ATOM | 878 | O | LEU | 61 | 8.148 | 2.160 | −3.220 | 1.00 | 0.00 |
| ATOM | 879 | CB | LEU | 61 | 5.293 | 3.307 | −3.797 | 1.00 | 0.00 |
| ATOM | 880 | CG | LEU | 61 | 5.465 | 4.029 | −2.533 | 1.00 | 0.00 |
| ATOM | 881 | CD1 | LEU | 61 | 5.648 | 5.537 | −2.726 | 1.00 | 0.00 |
| ATOM | 882 | CD2 | LEU | 61 | 4.216 | 3.758 | −1.693 | 1.00 | 0.00 |
| ATOM | 883 | HA | LEU | 61 | 7.069 | 4.104 | −4.660 | 1.00 | 0.00 |
| ATOM | 884 | HB3 | LEU | 61 | 5.001 | 2.273 | −3.609 | 1.00 | 0.00 |
| ATOM | 885 | HB2 | LEU | 61 | 4.398 | 3.727 | −4.256 | 1.00 | 0.00 |
| ATOM | 886 | HG | LEU | 61 | 6.348 | 3.623 | −2.037 | 1.00 | 0.00 |
| ATOM | 887 | HD21 | LEU | 61 | 3.987 | 2.692 | −1.690 | 1.00 | 0.00 |
| ATOM | 888 | HD22 | LEU | 61 | 4.272 | 4.118 | −0.666 | 1.00 | 0.00 |
| ATOM | 889 | HD23 | LEU | 61 | 3.354 | 4.114 | −2.257 | 1.00 | 0.00 |
| ATOM | 890 | HD11 | LEU | 61 | 6.383 | 5.762 | −3.498 | 1.00 | 0.00 |
| ATOM | 891 | HD12 | LEU | 61 | 4.672 | 5.997 | −2.881 | 1.00 | 0.00 |
| ATOM | 892 | HD13 | LEU | 61 | 6.111 | 6.002 | −1.857 | 1.00 | 0.00 |
| ATOM | 893 | H | LEU | 61 | 5.469 | 2.196 | −6.353 | 1.00 | 0.00 |
| ATOM | 894 | N | VAL | 62 | 7.477 | 0.903 | −4.949 | 1.00 | 0.00 |
| ATOM | 895 | CA | VAL | 62 | 8.144 | −0.367 | −4.603 | 1.00 | 0.00 |
| ATOM | 896 | C | VAL | 62 | 9.518 | −0.253 | −4.010 | 1.00 | 0.00 |
| ATOM | 897 | O | VAL | 62 | 9.842 | −1.093 | −3.151 | 1.00 | 0.00 |
| ATOM | 898 | CB | VAL | 62 | 8.188 | −1.150 | −5.958 | 1.00 | 0.00 |
| ATOM | 899 | CG1 | VAL | 62 | 9.153 | −0.589 | −7.044 | 1.00 | 0.00 |
| ATOM | 900 | CG2 | VAL | 62 | 8.371 | −2.711 | −5.801 | 1.00 | 0.00 |
| ATOM | 901 | HA | VAL | 62 | 7.476 | −0.812 | −3.865 | 1.00 | 0.00 |
| ATOM | 902 | HB | VAL | 62 | 7.203 | −1.052 | −6.414 | 1.00 | 0.00 |
| ATOM | 903 | HG11 | VAL | 62 | 10.165 | −0.547 | −6.641 | 1.00 | 0.00 |
| ATOM | 904 | HG12 | VAL | 62 | 9.073 | −1.204 | −7.941 | 1.00 | 0.00 |
| ATOM | 905 | HG13 | VAL | 62 | 8.883 | 0.453 | −7.218 | 1.00 | 0.00 |
| ATOM | 906 | HG21 | VAL | 62 | 9.231 | −2.937 | −5.171 | 1.00 | 0.00 |
| ATOM | 907 | HG22 | VAL | 62 | 7.424 | −3.033 | −5.367 | 1.00 | 0.00 |
| ATOM | 908 | HG23 | VAL | 62 | 8.551 | −3.249 | −6.731 | 1.00 | 0.00 |
| ATOM | 909 | H | VAL | 62 | 6.876 | 0.916 | −5.759 | 1.00 | 0.00 |
| ATOM | 910 | N | GLY | 63 | 10.288 | 0.756 | −4.394 | 1.00 | 0.00 |
| ATOM | 911 | CA | GLY | 63 | 11.671 | 0.994 | −3.968 | 1.00 | 0.00 |
| ATOM | 912 | C | GLY | 63 | 11.887 | 2.041 | −2.923 | 1.00 | 0.00 |
| ATOM | 913 | O | GLY | 63 | 13.047 | 2.199 | −2.436 | 1.00 | 0.00 |
| ATOM | 914 | HA3 | GLY | 63 | 12.225 | 1.316 | −4.850 | 1.00 | 0.00 |
| ATOM | 915 | HA2 | GLY | 63 | 12.165 | 0.082 | −3.632 | 1.00 | 0.00 |
| ATOM | 916 | H | GLY | 63 | 9.905 | 1.480 | −4.986 | 1.00 | 0.00 |
| ATOM | 917 | N | LEU | 64 | 10.805 | 2.603 | −2.445 | 1.00 | 0.00 |
| ATOM | 918 | CA | LEU | 64 | 10.796 | 3.505 | −1.268 | 1.00 | 0.00 |
| ATOM | 919 | C | LEU | 64 | 10.942 | 2.742 | 0.077 | 1.00 | 0.00 |
| ATOM | 920 | O | LEU | 64 | 10.473 | 1.615 | 0.190 | 1.00 | 0.00 |
| ATOM | 921 | CB | LEU | 64 | 9.727 | 4.625 | −1.242 | 1.00 | 0.00 |
| ATOM | 922 | CG | LEU | 64 | 9.617 | 5.501 | −2.473 | 1.00 | 0.00 |
| ATOM | 923 | CD1 | LEU | 64 | 8.999 | 6.839 | −2.065 | 1.00 | 0.00 |
| ATOM | 924 | CD2 | LEU | 64 | 10.942 | 5.782 | −3.213 | 1.00 | 0.00 |
| ATOM | 925 | HA | LEU | 64 | 11.756 | 4.017 | −1.233 | 1.00 | 0.00 |
| ATOM | 926 | HB3 | LEU | 64 | 9.941 | 5.259 | −0.381 | 1.00 | 0.00 |
| ATOM | 927 | HB2 | LEU | 64 | 8.729 | 4.247 | −1.019 | 1.00 | 0.00 |
| ATOM | 928 | HG | LEU | 64 | 8.959 | 4.974 | −3.163 | 1.00 | 0.00 |
| ATOM | 929 | HD21 | LEU | 64 | 11.485 | 6.503 | −2.603 | 1.00 | 0.00 |
| ATOM | 930 | HD22 | LEU | 64 | 11.519 | 4.875 | −3.389 | 1.00 | 0.00 |
| ATOM | 931 | HD23 | LEU | 64 | 10.847 | 6.249 | −4.194 | 1.00 | 0.00 |
| ATOM | 932 | HD11 | LEU | 64 | 9.574 | 7.492 | −1.407 | 1.00 | 0.00 |
| ATOM | 933 | HD12 | LEU | 64 | 8.879 | 7.378 | −3.004 | 1.00 | 0.00 |
| ATOM | 934 | HD13 | LEU | 64 | 8.045 | 6.781 | −1.540 | 1.00 | 0.00 |
| ATOM | 935 | H | LEU | 64 | 9.941 | 2.338 | −2.896 | 1.00 | 0.00 |
| ATOM | 936 | N | SER | 65 | 11.602 | 3.332 | 1.076 | 1.00 | 0.00 |
| ATOM | 937 | CA | SER | 65 | 11.591 | 2.806 | 2.469 | 1.00 | 0.00 |
| ATOM | 938 | C | SER | 65 | 10.165 | 2.793 | 3.049 | 1.00 | 0.00 |
| ATOM | 939 | O | SER | 65 | 9.395 | 3.708 | 2.842 | 1.00 | 0.00 |
| ATOM | 940 | CB | SER | 65 | 12.545 | 3.603 | 3.367 | 1.00 | 0.00 |
| ATOM | 941 | OG | SER | 65 | 13.729 | 3.803 | 2.733 | 1.00 | 0.00 |
| ATOM | 942 | HA | SER | 65 | 11.952 | 1.786 | 2.339 | 1.00 | 0.00 |
| ATOM | 943 | HB3 | SER | 65 | 12.823 | 2.951 | 4.194 | 1.00 | 0.00 |
| ATOM | 944 | HB2 | SER | 65 | 12.138 | 4.554 | 3.709 | 1.00 | 0.00 |
| ATOM | 945 | HG | SER | 65 | 14.359 | 4.089 | 3.399 | 1.00 | 0.00 |
| ATOM | 946 | H | SER | 65 | 12.037 | 4.232 | 0.933 | 1.00 | 0.00 |
| ATOM | 947 | N | GLN | 66 | 9.817 | 1.843 | 3.964 | 1.00 | 0.00 |
| ATOM | 948 | CA | GLN | 66 | 8.451 | 1.920 | 4.529 | 1.00 | 0.00 |
| ATOM | 949 | C | GLN | 66 | 8.174 | 3.139 | 5.264 | 1.00 | 0.00 |
| ATOM | 950 | O | GLN | 66 | 7.116 | 3.650 | 5.193 | 1.00 | 0.00 |
| ATOM | 951 | CB | GLN | 66 | 8.096 | 0.634 | 5.312 | 1.00 | 0.00 |
| ATOM | 952 | CG | GLN | 66 | 8.919 | 0.369 | 6.600 | 1.00 | 0.00 |
| ATOM | 953 | CD | GLN | 66 | 8.652 | 1.378 | 7.745 | 1.00 | 0.00 |
| ATOM | 954 | OE1 | GLN | 66 | 7.474 | 1.565 | 8.157 | 1.00 | 0.00 |
| ATOM | 955 | NE2 | GLN | 66 | 9.691 | 1.985 | 8.201 | 1.00 | 0.00 |
| ATOM | 956 | HA | GLN | 66 | 7.829 | 1.901 | 3.634 | 1.00 | 0.00 |
| ATOM | 957 | HB3 | GLN | 66 | 8.323 | −0.183 | 4.627 | 1.00 | 0.00 |
| ATOM | 958 | HB2 | GLN | 66 | 7.066 | 0.688 | 5.668 | 1.00 | 0.00 |
| ATOM | 959 | HG3 | GLN | 66 | 9.959 | 0.265 | 6.291 | 1.00 | 0.00 |
| ATOM | 960 | HG2 | GLN | 66 | 8.653 | −0.639 | 6.922 | 1.00 | 0.00 |
| ATOM | 961 | HE22 | GLN | 66 | 10.576 | 1.942 | 7.714 | 1.00 | 0.00 |
| ATOM | 962 | HE21 | GLN | 66 | 9.532 | 2.541 | 9.028 | 1.00 | 0.00 |
| ATOM | 963 | H | GLN | 66 | 10.469 | 1.079 | 3.860 | 1.00 | 0.00 |
| ATOM | 964 | N | GLU | 67 | 9.156 | 3.774 | 5.833 | 1.00 | 0.00 |
| ATOM | 965 | CA | GLU | 67 | 9.018 | 5.050 | 6.522 | 1.00 | 0.00 |
| ATOM | 966 | C | GLU | 67 | 8.828 | 6.254 | 5.626 | 1.00 | 0.00 |
| ATOM | 967 | O | GLU | 67 | 8.172 | 7.135 | 6.157 | 1.00 | 0.00 |
| ATOM | 968 | CB | GLU | 67 | 10.235 | 5.268 | 7.403 | 1.00 | 0.00 |
| ATOM | 969 | CG | GLU | 67 | 11.580 | 5.251 | 6.660 | 1.00 | 0.00 |
| ATOM | 970 | CD | GLU | 67 | 12.347 | 3.879 | 6.661 | 1.00 | 0.00 |
| ATOM | 971 | OE1 | GLU | 67 | 11.780 | 2.803 | 6.484 | 1.00 | 0.00 |
| ATOM | 972 | OE2 | GLU | 67 | 13.625 | 3.843 | 6.646 | 1.00 | 0.00 |
| ATOM | 973 | HA | GLU | 67 | 8.223 | 4.928 | 7.258 | 1.00 | 0.00 |
| ATOM | 974 | HB3 | GLU | 67 | 10.244 | 4.598 | 8.262 | 1.00 | 0.00 |
| ATOM | 975 | HB2 | GLU | 67 | 10.174 | 6.230 | 7.913 | 1.00 | 0.00 |
| ATOM | 976 | HG3 | GLU | 67 | 12.244 | 5.954 | 7.162 | 1.00 | 0.00 |
| ATOM | 977 | HG2 | GLU | 67 | 11.404 | 5.571 | 5.633 | 1.00 | 0.00 |
| ATOM | 978 | H | GLU | 67 | 10.050 | 3.304 | 5.798 | 1.00 | 0.00 |
| ATOM | 979 | N | ARG | 68 | 9.124 | 6.261 | 4.320 | 1.00 | 0.00 |
| ATOM | 980 | CA | ARG | 68 | 8.794 | 7.291 | 3.297 | 1.00 | 0.00 |
| ATOM | 981 | C | ARG | 68 | 7.505 | 6.864 | 2.566 | 1.00 | 0.00 |
| ATOM | 982 | O | ARG | 68 | 6.621 | 7.680 | 2.304 | 1.00 | 0.00 |
| ATOM | 983 | CB | ARG | 68 | 10.007 | 7.505 | 2.356 | 1.00 | 0.00 |
| ATOM | 984 | CG | ARG | 68 | 10.685 | 8.924 | 2.481 | 1.00 | 0.00 |
| ATOM | 985 | CD | ARG | 68 | 11.439 | 9.383 | 3.706 | 1.00 | 0.00 |
| ATOM | 986 | NE | ARG | 68 | 10.438 | 9.587 | 4.725 | 1.00 | 0.00 |
| ATOM | 987 | CZ | ARG | 68 | 10.563 | 9.573 | 6.002 | 1.00 | 0.00 |
| ATOM | 988 | NH1 | ARG | 68 | 9.635 | 10.018 | 6.737 | 1.00 | 0.00 |
| ATOM | 989 | NH2 | ARG | 68 | 11.620 | 9.044 | 6.623 | 1.00 | 0.00 |
| ATOM | 990 | HA | ARG | 68 | 8.538 | 8.239 | 3.772 | 1.00 | 0.00 |
| ATOM | 991 | HB3 | ARG | 68 | 9.690 | 7.412 | 1.317 | 1.00 | 0.00 |
| ATOM | 992 | HB2 | ARG | 68 | 10.799 | 6.756 | 2.351 | 1.00 | 0.00 |
| ATOM | 993 | HG3 | ARG | 68 | 9.894 | 9.595 | 2.144 | 1.00 | 0.00 |
| ATOM | 994 | HG2 | ARG | 68 | 11.486 | 8.943 | 1.743 | 1.00 | 0.00 |
| ATOM | 995 | HD3 | ARG | 68 | 11.957 | 10.317 | 3.490 | 1.00 | 0.00 |
| ATOM | 996 | HD2 | ARG | 68 | 12.107 | 8.618 | 4.101 | 1.00 | 0.00 |
| ATOM | 997 | HE | ARG | 68 | 9.626 | 10.100 | 4.412 | 1.00 | 0.00 |
| ATOM | 998 | HH12 | ARG | 68 | 9.791 | 10.078 | 7.733 | 1.00 | 0.00 |
| ATOM | 999 | HH11 | ARG | 68 | 8.756 | 10.276 | 6.313 | 1.00 | 0.00 |
| ATOM | 1000 | HH22 | ARG | 68 | 11.690 | 8.916 | 7.622 | 1.00 | 0.00 |
| ATOM | 1001 | HH21 | ARG | 68 | 12.341 | 8.696 | 6.008 | 1.00 | 0.00 |
| ATOM | 1002 | H | ARG | 68 | 9.599 | 5.413 | 4.048 | 1.00 | 0.00 |
| ATOM | 1003 | N | ALA | 69 | 7.286 | 5.603 | 2.282 | 1.00 | 0.00 |
| ATOM | 1004 | CA | ALA | 69 | 6.104 | 4.975 | 1.663 | 1.00 | 0.00 |
| ATOM | 1005 | C | ALA | 69 | 4.775 | 5.026 | 2.495 | 1.00 | 0.00 |
| ATOM | 1006 | O | ALA | 69 | 3.821 | 5.624 | 1.959 | 1.00 | 0.00 |
| ATOM | 1007 | CB | ALA | 69 | 6.406 | 3.492 | 1.265 | 1.00 | 0.00 |
| ATOM | 1008 | HA | ALA | 69 | 5.852 | 5.522 | 0.756 | 1.00 | 0.00 |
| ATOM | 1009 | HB1 | ALA | 69 | 6.870 | 3.330 | 0.292 | 1.00 | 0.00 |
| ATOM | 1010 | HB2 | ALA | 69 | 6.957 | 2.907 | 2.001 | 1.00 | 0.00 |
| ATOM | 1011 | HB3 | ALA | 69 | 5.431 | 3.007 | 1.206 | 1.00 | 0.00 |
| ATOM | 1012 | H | ALA | 69 | 8.085 | 4.985 | 2.306 | 1.00 | 0.00 |
| ATOM | 1013 | N | ALA | 70 | 4.720 | 4.464 | 3.776 | 1.00 | 0.00 |
| ATOM | 1014 | CA | ALA | 70 | 3.527 | 4.643 | 4.547 | 1.00 | 0.00 |
| ATOM | 1015 | C | ALA | 70 | 3.209 | 6.184 | 4.827 | 1.00 | 0.00 |
| ATOM | 1016 | O | ALA | 70 | 2.073 | 6.492 | 5.035 | 1.00 | 0.00 |
| ATOM | 1017 | CB | ALA | 70 | 3.643 | 3.874 | 5.822 | 1.00 | 0.00 |
| ATOM | 1018 | HA | ALA | 70 | 2.730 | 4.217 | 3.938 | 1.00 | 0.00 |
| ATOM | 1019 | HB1 | ALA | 70 | 4.492 | 4.323 | 6.337 | 1.00 | 0.00 |
| ATOM | 1020 | HB2 | ALA | 70 | 2.695 | 3.948 | 6.357 | 1.00 | 0.00 |
| ATOM | 1021 | HB3 | ALA | 70 | 3.773 | 2.821 | 5.574 | 1.00 | 0.00 |
| ATOM | 1022 | H | ALA | 70 | 5.532 | 4.063 | 4.222 | 1.00 | 0.00 |
| ATOM | 1023 | N | GLU | 71 | 4.210 | 7.112 | 4.795 | 1.00 | 0.00 |
| ATOM | 1024 | CA | GLU | 71 | 4.112 | 8.540 | 4.870 | 1.00 | 0.00 |
| ATOM | 1025 | C | GLU | 71 | 3.388 | 9.163 | 3.644 | 1.00 | 0.00 |
| ATOM | 1026 | O | GLU | 71 | 2.455 | 9.933 | 3.765 | 1.00 | 0.00 |
| ATOM | 1027 | CB | GLU | 71 | 5.501 | 9.048 | 5.220 | 1.00 | 0.00 |
| ATOM | 1028 | CG | GLU | 71 | 5.555 | 10.640 | 5.083 | 1.00 | 0.00 |
| ATOM | 1029 | CD | GLU | 71 | 7.006 | 11.083 | 5.265 | 1.00 | 0.00 |
| ATOM | 1030 | OE1 | GLU | 71 | 7.315 | 11.388 | 6.413 | 1.00 | 0.00 |
| ATOM | 1031 | OE2 | GLU | 71 | 7.812 | 10.826 | 4.399 | 1.00 | 0.00 |
| ATOM | 1032 | HA | GLU | 71 | 3.507 | 8.790 | 5.741 | 1.00 | 0.00 |
| ATOM | 1033 | HB3 | GLU | 71 | 6.331 | 8.563 | 4.708 | 1.00 | 0.00 |
| ATOM | 1034 | HB2 | GLU | 71 | 5.682 | 8.898 | 6.285 | 1.00 | 0.00 |
| ATOM | 1035 | HG3 | GLU | 71 | 4.835 | 11.096 | 5.763 | 1.00 | 0.00 |
| ATOM | 1036 | HG2 | GLU | 71 | 5.237 | 10.993 | 4.103 | 1.00 | 0.00 |
| ATOM | 1037 | H | GLU | 71 | 5.153 | 6.759 | 4.719 | 1.00 | 0.00 |
| ATOM | 1038 | N | LEU | 72 | 3.790 | 8.809 | 2.438 | 1.00 | 0.00 |
| ATOM | 1039 | CA | LEU | 72 | 3.159 | 9.288 | 1.207 | 1.00 | 0.00 |
| ATOM | 1040 | C | LEU | 72 | 1.702 | 8.762 | 1.189 | 1.00 | 0.00 |
| ATOM | 1041 | O | LEU | 72 | 0.722 | 9.434 | 0.984 | 1.00 | 0.00 |
| ATOM | 1042 | CB | LEU | 72 | 4.021 | 8.840 | 0.032 | 1.00 | 0.00 |
| ATOM | 1043 | CG | LEU | 72 | 3.540 | 9.478 | −1.266 | 1.00 | 0.00 |
| ATOM | 1044 | CD1 | LEU | 72 | 3.728 | 10.995 | −1.387 | 1.00 | 0.00 |
| ATOM | 1045 | CD2 | LEU | 72 | 4.358 | 8.986 | −2.467 | 1.00 | 0.00 |
| ATOM | 1046 | HA | LEU | 72 | 3.188 | 10.378 | 1.211 | 1.00 | 0.00 |
| ATOM | 1047 | HB3 | LEU | 72 | 3.948 | 7.776 | −0.193 | 1.00 | 0.00 |
| ATOM | 1048 | HB2 | LEU | 72 | 5.072 | 9.094 | 0.166 | 1.00 | 0.00 |
| ATOM | 1049 | HG | LEU | 72 | 2.492 | 9.251 | −1.462 | 1.00 | 0.00 |
| ATOM | 1050 | HD21 | LEU | 72 | 4.212 | 7.907 | −2.523 | 1.00 | 0.00 |
| ATOM | 1051 | HD22 | LEU | 72 | 4.081 | 9.416 | −3.430 | 1.00 | 0.00 |
| ATOM | 1052 | HD23 | LEU | 72 | 5.435 | 9.141 | −2.397 | 1.00 | 0.00 |
| ATOM | 1053 | HD11 | LEU | 72 | 4.417 | 11.401 | −0.646 | 1.00 | 0.00 |
| ATOM | 1054 | HD12 | LEU | 72 | 4.114 | 11.315 | −2.354 | 1.00 | 0.00 |
| ATOM | 1055 | HD13 | LEU | 72 | 2.771 | 11.490 | −1.213 | 1.00 | 0.00 |
| ATOM | 1056 | H | LEU | 72 | 4.676 | 8.327 | 2.388 | 1.00 | 0.00 |
| ATOM | 1057 | N | MET | 73 | 1.525 | 7.488 | 1.496 | 1.00 | 0.00 |
| ATOM | 1058 | CA | MET | 73 | 0.286 | 6.798 | 1.758 | 1.00 | 0.00 |
| ATOM | 1059 | C | MET | 73 | −0.564 | 7.251 | 2.958 | 1.00 | 0.00 |
| ATOM | 1060 | O | MET | 73 | −1.703 | 6.849 | 3.082 | 1.00 | 0.00 |
| ATOM | 1061 | CB | MET | 73 | 0.636 | 5.312 | 1.801 | 1.00 | 0.00 |
| ATOM | 1062 | CG | MET | 73 | 1.177 | 4.817 | 0.477 | 1.00 | 0.00 |
| ATOM | 1063 | SD | MET | 73 | 1.091 | 3.052 | 0.271 | 1.00 | 0.00 |
| ATOM | 1064 | CE | MET | 73 | 2.566 | 2.461 | 1.159 | 1.00 | 0.00 |
| ATOM | 1065 | HA | MET | 73 | −0.361 | 6.964 | 0.896 | 1.00 | 0.00 |
| ATOM | 1066 | HB3 | MET | 73 | −0.313 | 4.845 | 2.067 | 1.00 | 0.00 |
| ATOM | 1067 | HB2 | MET | 73 | 1.259 | 5.025 | 2.648 | 1.00 | 0.00 |
| ATOM | 1068 | HG3 | MET | 73 | 2.219 | 5.136 | 0.423 | 1.00 | 0.00 |
| ATOM | 1069 | HG2 | MET | 73 | 0.667 | 5.386 | −0.301 | 1.00 | 0.00 |
| ATOM | 1070 | HE1 | MET | 73 | 2.553 | 1.390 | 1.358 | 1.00 | 0.00 |
| ATOM | 1071 | HE2 | MET | 73 | 3.485 | 2.612 | 0.590 | 1.00 | 0.00 |
| ATOM | 1072 | HE3 | MET | 73 | 2.732 | 3.069 | 2.048 | 1.00 | 0.00 |
| ATOM | 1073 | H | MET | 73 | 2.310 | 6.892 | 1.716 | 1.00 | 0.00 |
| ATOM | 1074 | N | THR | 74 | −0.054 | 7.974 | 3.943 | 1.00 | 0.00 |
| ATOM | 1075 | CA | THR | 74 | −0.716 | 8.746 | 4.973 | 1.00 | 0.00 |
| ATOM | 1076 | C | THR | 74 | −1.037 | 10.210 | 4.519 | 1.00 | 0.00 |
| ATOM | 1077 | O | THR | 74 | −2.161 | 10.608 | 4.804 | 1.00 | 0.00 |
| ATOM | 1078 | CB | THR | 74 | 0.212 | 8.707 | 6.231 | 1.00 | 0.00 |
| ATOM | 1079 | OG1 | THR | 74 | 0.252 | 7.362 | 6.693 | 1.00 | 0.00 |
| ATOM | 1080 | CG2 | THR | 74 | −0.216 | 9.665 | 7.397 | 1.00 | 0.00 |
| ATOM | 1081 | HA | THR | 74 | −1.592 | 8.180 | 5.290 | 1.00 | 0.00 |
| ATOM | 1082 | HB | THR | 74 | 1.237 | 8.961 | 5.962 | 1.00 | 0.00 |
| ATOM | 1083 | HG1 | THR | 74 | 0.928 | 6.947 | 6.152 | 1.00 | 0.00 |
| ATOM | 1084 | HG23 | THR | 74 | −0.345 | 10.709 | 7.110 | 1.00 | 0.00 |
| ATOM | 1085 | HG21 | THR | 74 | −1.205 | 9.361 | 7.738 | 1.00 | 0.00 |
| ATOM | 1086 | HG22 | THR | 74 | 0.479 | 9.477 | 8.215 | 1.00 | 0.00 |
| ATOM | 1087 | H | THR | 74 | 0.951 | 8.064 | 3.982 | 1.00 | 0.00 |
| ATOM | 1088 | N | ARG | 75 | −0.149 | 10.893 | 3.759 | 1.00 | 0.00 |
| ATOM | 1089 | CA | ARG | 75 | −0.276 | 12.331 | 3.322 | 1.00 | 0.00 |
| ATOM | 1090 | C | ARG | 75 | −0.777 | 12.603 | 1.864 | 1.00 | 0.00 |
| ATOM | 1091 | O | ARG | 75 | −1.071 | 13.794 | 1.490 | 1.00 | 0.00 |
| ATOM | 1092 | CB | ARG | 75 | 0.970 | 13.162 | 3.530 | 1.00 | 0.00 |
| ATOM | 1093 | CG | ARG | 75 | 1.274 | 13.288 | 5.010 | 1.00 | 0.00 |
| ATOM | 1094 | CD | ARG | 75 | 2.574 | 14.016 | 5.209 | 1.00 | 0.00 |
| ATOM | 1095 | NE | ARG | 75 | 3.186 | 13.644 | 6.520 | 1.00 | 0.00 |
| ATOM | 1096 | CZ | ARG | 75 | 4.421 | 13.875 | 7.001 | 1.00 | 0.00 |
| ATOM | 1097 | NH1 | ARG | 75 | 4.723 | 13.309 | 8.107 | 1.00 | 0.00 |
| ATOM | 1098 | NH2 | ARG | 75 | 5.306 | 14.556 | 6.359 | 1.00 | 0.00 |
| ATOM | 1099 | HA | ARG | 75 | −1.059 | 12.614 | 4.025 | 1.00 | 0.00 |
| ATOM | 1100 | HB3 | ARG | 75 | 0.852 | 14.193 | 3.196 | 1.00 | 0.00 |
| ATOM | 1101 | HB2 | ARG | 75 | 1.787 | 12.734 | 2.947 | 1.00 | 0.00 |
| ATOM | 1102 | HG3 | ARG | 75 | 1.460 | 12.330 | 5.494 | 1.00 | 0.00 |
| ATOM | 1103 | HG2 | ARG | 75 | 0.465 | 13.743 | 5.582 | 1.00 | 0.00 |
| ATOM | 1104 | HD3 | ARG | 75 | 2.314 | 15.052 | 5.421 | 1.00 | 0.00 |
| ATOM | 1105 | HD2 | ARG | 75 | 3.211 | 13.865 | 4.338 | 1.00 | 0.00 |
| ATOM | 1106 | HE | ARG | 75 | 2.719 | 12.899 | 7.016 | 1.00 | 0.00 |
| ATOM | 1107 | HH12 | ARG | 75 | 5.670 | 13.287 | 8.456 | 1.00 | 0.00 |
| ATOM | 1108 | HH11 | ARG | 75 | 4.027 | 12.688 | 8.497 | 1.00 | 0.00 |
| ATOM | 1109 | HH22 | ARG | 75 | 6.232 | 14.506 | 6.758 | 1.00 | 0.00 |
| ATOM | 1110 | HH21 | ARG | 75 | 5.062 | 15.040 | 5.507 | 1.00 | 0.00 |
| ATOM | 1111 | H | ARG | 75 | 0.701 | 10.372 | 3.597 | 1.00 | 0.00 |
| ATOM | 1112 | N | THR | 76 | −1.049 | 11.578 | 1.052 | 1.00 | 0.00 |
| ATOM | 1113 | CA | THR | 76 | −1.785 | 11.709 | −0.202 | 1.00 | 0.00 |
| ATOM | 1114 | C | THR | 76 | −3.294 | 12.052 | −0.039 | 1.00 | 0.00 |
| ATOM | 1115 | O | THR | 76 | −3.784 | 12.413 | 1.041 | 1.00 | 0.00 |
| ATOM | 1116 | CB | THR | 76 | −1.547 | 10.408 | −0.979 | 1.00 | 0.00 |
| ATOM | 1117 | OG1 | THR | 76 | −1.912 | 10.672 | −2.287 | 1.00 | 0.00 |
| ATOM | 1118 | CG2 | THR | 76 | −2.384 | 9.261 | −0.576 | 1.00 | 0.00 |
| ATOM | 1119 | HA | THR | 76 | −1.351 | 12.521 | −0.786 | 1.00 | 0.00 |
| ATOM | 1120 | HB | THR | 76 | −0.473 | 10.222 | −0.990 | 1.00 | 0.00 |
| ATOM | 1121 | HG1 | THR | 76 | −1.097 | 10.951 | −2.709 | 1.00 | 0.00 |
| ATOM | 1122 | HG23 | THR | 76 | −2.145 | 9.060 | 0.468 | 1.00 | 0.00 |
| ATOM | 1123 | HG21 | THR | 76 | −3.436 | 9.531 | −0.666 | 1.00 | 0.00 |
| ATOM | 1124 | HG22 | THR | 76 | −2.064 | 8.384 | −1.136 | 1.00 | 0.00 |
| ATOM | 1125 | H | THR | 76 | −0.760 | 10.670 | 1.389 | 1.00 | 0.00 |
| ATOM | 1126 | N | SER | 77 | −4.163 | 11.917 | −1.012 | 1.00 | 0.00 |
| ATOM | 1127 | CA | SER | 77 | −5.580 | 12.379 | −0.911 | 1.00 | 0.00 |
| ATOM | 1128 | C | SER | 77 | −6.600 | 11.309 | −1.155 | 1.00 | 0.00 |
| ATOM | 1129 | O | SER | 77 | −6.231 | 10.267 | −1.718 | 1.00 | 0.00 |
| ATOM | 1130 | CB | SER | 77 | −5.696 | 13.573 | −1.855 | 1.00 | 0.00 |
| ATOM | 1131 | OG | SER | 77 | −5.127 | 13.350 | −3.193 | 1.00 | 0.00 |
| ATOM | 1132 | HA | SER | 77 | −5.824 | 12.794 | 0.067 | 1.00 | 0.00 |
| ATOM | 1133 | HB3 | SER | 77 | −5.126 | 14.419 | −1.473 | 1.00 | 0.00 |
| ATOM | 1134 | HB2 | SER | 77 | −6.764 | 13.791 | −1.901 | 1.00 | 0.00 |
| ATOM | 1135 | HG | SER | 77 | −4.582 | 14.122 | −3.358 | 1.00 | 0.00 |
| ATOM | 1136 | H | SER | 77 | −3.844 | 11.454 | −1.850 | 1.00 | 0.00 |
| ATOM | 1137 | N | SER | 78 | −7.842 | 11.509 | −0.713 | 1.00 | 0.00 |
| ATOM | 1138 | CA | SER | 78 | −8.953 | 10.581 | −0.634 | 1.00 | 0.00 |
| ATOM | 1139 | C | SER | 78 | −9.550 | 10.128 | −2.010 | 1.00 | 0.00 |
| ATOM | 1140 | O | SER | 78 | −10.708 | 9.656 | −2.066 | 1.00 | 0.00 |
| ATOM | 1141 | CB | SER | 78 | −10.058 | 11.226 | 0.263 | 1.00 | 0.00 |
| ATOM | 1142 | OG | SER | 78 | −9.512 | 11.690 | 1.484 | 1.00 | 0.00 |
| ATOM | 1143 | HA | SER | 78 | −8.568 | 9.671 | −0.172 | 1.00 | 0.00 |
| ATOM | 1144 | HB3 | SER | 78 | −10.804 | 10.480 | 0.538 | 1.00 | 0.00 |
| ATOM | 1145 | HB2 | SER | 78 | −10.394 | 12.137 | −0.233 | 1.00 | 0.00 |
| ATOM | 1146 | HG | SER | 78 | −10.165 | 12.239 | 1.925 | 1.00 | 0.00 |
| ATOM | 1147 | H | SER | 78 | −8.096 | 12.351 | −0.219 | 1.00 | 0.00 |
| ATOM | 1148 | N | VAL | 79 | −8.707 | 10.212 | −3.125 | 1.00 | 0.00 |
| ATOM | 1149 | CA | VAL | 79 | −9.114 | 9.784 | −4.528 | 1.00 | 0.00 |
| ATOM | 1150 | C | VAL | 79 | −8.007 | 9.075 | −5.274 | 1.00 | 0.00 |
| ATOM | 1151 | O | VAL | 79 | −8.249 | 8.539 | −6.343 | 1.00 | 0.00 |
| ATOM | 1152 | CB | VAL | 79 | −9.623 | 10.965 | −5.440 | 1.00 | 0.00 |
| ATOM | 1153 | CG1 | VAL | 79 | −10.723 | 10.523 | −6.452 | 1.00 | 0.00 |
| ATOM | 1154 | CG2 | VAL | 79 | −10.218 | 12.114 | −4.613 | 1.00 | 0.00 |
| ATOM | 1155 | HA | VAL | 79 | −9.842 | 8.978 | −4.437 | 1.00 | 0.00 |
| ATOM | 1156 | HB | VAL | 79 | −8.771 | 11.434 | −5.935 | 1.00 | 0.00 |
| ATOM | 1157 | HG11 | VAL | 79 | −10.338 | 9.806 | −7.178 | 1.00 | 0.00 |
| ATOM | 1158 | HG12 | VAL | 79 | −11.501 | 10.019 | −5.877 | 1.00 | 0.00 |
| ATOM | 1159 | HG13 | VAL | 79 | −11.124 | 11.463 | −6.828 | 1.00 | 0.00 |
| ATOM | 1160 | HG21 | VAL | 79 | −10.983 | 11.625 | −4.011 | 1.00 | 0.00 |
| ATOM | 1161 | HG22 | VAL | 79 | −9.503 | 12.554 | −3.918 | 1.00 | 0.00 |
| ATOM | 1162 | HG23 | VAL | 79 | −10.623 | 12.867 | −5.290 | 1.00 | 0.00 |
| ATOM | 1163 | H | VAL | 79 | −7.767 | 10.564 | −3.006 | 1.00 | 0.00 |
| ATOM | 1164 | N | VAL | 80 | −6.773 | 9.041 | −4.827 | 1.00 | 0.00 |
| ATOM | 1165 | CA | VAL | 80 | −5.645 | 8.379 | −5.551 | 1.00 | 0.00 |
| ATOM | 1166 | C | VAL | 80 | −5.787 | 6.843 | −5.617 | 1.00 | 0.00 |
| ATOM | 1167 | O | VAL | 80 | −6.396 | 6.292 | −4.732 | 1.00 | 0.00 |
| ATOM | 1168 | CB | VAL | 80 | −4.266 | 8.763 | −4.942 | 1.00 | 0.00 |
| ATOM | 1169 | CG1 | VAL | 80 | −3.957 | 10.267 | −4.801 | 1.00 | 0.00 |
| ATOM | 1170 | CG2 | VAL | 80 | −4.051 | 8.062 | −3.610 | 1.00 | 0.00 |
| ATOM | 1171 | HA | VAL | 80 | −5.610 | 8.847 | −6.535 | 1.00 | 0.00 |
| ATOM | 1172 | HB | VAL | 80 | −3.531 | 8.347 | −5.631 | 1.00 | 0.00 |
| ATOM | 1173 | HG11 | VAL | 80 | −4.321 | 10.854 | −5.644 | 1.00 | 0.00 |
| ATOM | 1174 | HG12 | VAL | 80 | −4.430 | 10.573 | −3.868 | 1.00 | 0.00 |
| ATOM | 1175 | HG13 | VAL | 80 | −2.873 | 10.375 | −4.747 | 1.00 | 0.00 |
| ATOM | 1176 | HG21 | VAL | 80 | −4.143 | 6.984 | −3.738 | 1.00 | 0.00 |
| ATOM | 1177 | HG22 | VAL | 80 | −3.007 | 8.283 | −3.387 | 1.00 | 0.00 |
| ATOM | 1178 | HG23 | VAL | 80 | −4.727 | 8.398 | −2.824 | 1.00 | 0.00 |
| ATOM | 1179 | H | VAL | 80 | −6.548 | 9.519 | −3.965 | 1.00 | 0.00 |
| ATOM | 1180 | N | THR | 81 | −5.239 | 6.285 | −6.723 | 1.00 | 0.00 |
| ATOM | 1181 | CA | THR | 81 | −5.116 | 4.803 | −6.878 | 1.00 | 0.00 |
| ATOM | 1182 | C | THR | 81 | −4.024 | 4.311 | −5.897 | 1.00 | 0.00 |
| ATOM | 1183 | O | THR | 81 | −2.957 | 4.942 | −5.711 | 1.00 | 0.00 |
| ATOM | 1184 | CB | THR | 81 | −4.693 | 4.453 | −8.305 | 1.00 | 0.00 |
| ATOM | 1185 | OG1 | THR | 81 | −3.570 | 5.271 | −8.786 | 1.00 | 0.00 |
| ATOM | 1186 | CG2 | THR | 81 | −5.874 | 4.747 | −9.354 | 1.00 | 0.00 |
| ATOM | 1187 | HA | THR | 81 | −5.949 | 4.195 | −6.524 | 1.00 | 0.00 |
| ATOM | 1188 | HB | THR | 81 | −4.498 | 3.392 | −8.459 | 1.00 | 0.00 |
| ATOM | 1189 | HG1 | THR | 81 | −2.744 | 4.849 | −8.539 | 1.00 | 0.00 |
| ATOM | 1190 | HG23 | THR | 81 | −6.727 | 4.176 | −8.987 | 1.00 | 0.00 |
| ATOM | 1191 | HG21 | THR | 81 | −6.153 | 5.794 | −9.229 | 1.00 | 0.00 |
| ATOM | 1192 | HG22 | THR | 81 | −5.442 | 4.342 | −10.268 | 1.00 | 0.00 |
| ATOM | 1193 | H | THR | 81 | −4.664 | 6.736 | −7.420 | 1.00 | 0.00 |
| ATOM | 1194 | N | LEU | 82 | −4.148 | 3.054 | −5.383 | 1.00 | 0.00 |
| ATOM | 1195 | CA | LEU | 82 | −3.139 | 2.218 | −4.693 | 1.00 | 0.00 |
| ATOM | 1196 | C | LEU | 82 | −3.172 | 0.766 | −5.309 | 1.00 | 0.00 |
| ATOM | 1197 | O | LEU | 82 | −4.240 | 0.318 | −5.716 | 1.00 | 0.00 |
| ATOM | 1198 | CB | LEU | 82 | −3.300 | 2.269 | −3.168 | 1.00 | 0.00 |
| ATOM | 1199 | CG | LEU | 82 | −4.706 | 1.918 | −2.680 | 1.00 | 0.00 |
| ATOM | 1200 | CD1 | LEU | 82 | −4.722 | 0.899 | −1.499 | 1.00 | 0.00 |
| ATOM | 1201 | CD2 | LEU | 82 | −5.505 | 3.078 | −2.185 | 1.00 | 0.00 |
| ATOM | 1202 | HA | LEU | 82 | −2.209 | 2.718 | −4.968 | 1.00 | 0.00 |
| ATOM | 1203 | HB3 | LEU | 82 | −3.090 | 3.267 | −2.782 | 1.00 | 0.00 |
| ATOM | 1204 | HB2 | LEU | 82 | −2.571 | 1.622 | −2.679 | 1.00 | 0.00 |
| ATOM | 1205 | HG | LEU | 82 | −5.183 | 1.439 | −3.536 | 1.00 | 0.00 |
| ATOM | 1206 | HD21 | LEU | 82 | −6.498 | 2.691 | −1.957 | 1.00 | 0.00 |
| ATOM | 1207 | HD22 | LEU | 82 | −5.084 | 3.672 | −1.373 | 1.00 | 0.00 |
| ATOM | 1208 | HD23 | LEU | 82 | −5.487 | 3.836 | −2.968 | 1.00 | 0.00 |
| ATOM | 1209 | HD11 | LEU | 82 | −4.013 | 0.131 | −1.807 | 1.00 | 0.00 |
| ATOM | 1210 | HD12 | LEU | 82 | −4.244 | 1.438 | −0.681 | 1.00 | 0.00 |
| ATOM | 1211 | HD13 | LEU | 82 | −5.683 | 0.458 | −1.235 | 1.00 | 0.00 |
| ATOM | 1212 | H | LEU | 82 | −4.968 | 2.545 | −5.680 | 1.00 | 0.00 |
| ATOM | 1213 | N | GLU | 83 | −1.994 | 0.189 | −5.536 | 1.00 | 0.00 |
| ATOM | 1214 | CA | GLU | 83 | −1.800 | −1.055 | −6.241 | 1.00 | 0.00 |
| ATOM | 1215 | C | GLU | 83 | −1.065 | −2.065 | −5.335 | 1.00 | 0.00 |
| ATOM | 1216 | O | GLU | 83 | 0.149 | −2.027 | −5.139 | 1.00 | 0.00 |
| ATOM | 1217 | CB | GLU | 83 | −0.951 | −0.763 | −7.486 | 1.00 | 0.00 |
| ATOM | 1218 | CG | GLU | 83 | −1.679 | 0.013 | −8.625 | 1.00 | 0.00 |
| ATOM | 1219 | CD | GLU | 83 | −1.653 | −0.606 | −10.080 | 1.00 | 0.00 |
| ATOM | 1220 | OE1 | GLU | 83 | −2.669 | −0.757 | −10.731 | 1.00 | 0.00 |
| ATOM | 1221 | OE2 | GLU | 83 | −0.598 | −1.067 | −10.591 | 1.00 | 0.00 |
| ATOM | 1222 | HA | GLU | 83 | −2.722 | −1.542 | −6.562 | 1.00 | 0.00 |
| ATOM | 1223 | HB3 | GLU | 83 | −0.497 | −1.693 | −7.825 | 1.00 | 0.00 |
| ATOM | 1224 | HB2 | GLU | 83 | −0.215 | −0.031 | −7.151 | 1.00 | 0.00 |
| ATOM | 1225 | HG3 | GLU | 83 | −1.281 | 1.019 | −8.762 | 1.00 | 0.00 |
| ATOM | 1226 | HG2 | GLU | 83 | −2.734 | 0.044 | −8.353 | 1.00 | 0.00 |
| ATOM | 1227 | H | GLU | 83 | −1.221 | 0.823 | −5.397 | 1.00 | 0.00 |
| ATOM | 1228 | N | VAL | 84 | −1.784 | −3.116 | −4.899 | 1.00 | 0.00 |
| ATOM | 1229 | CA | VAL | 84 | −1.440 | −4.029 | −3.773 | 1.00 | 0.00 |
| ATOM | 1230 | C | VAL | 84 | −1.723 | −5.482 | −4.181 | 1.00 | 0.00 |
| ATOM | 1231 | O | VAL | 84 | −2.443 | −5.825 | −5.093 | 1.00 | 0.00 |
| ATOM | 1232 | CB | VAL | 84 | −2.182 | −3.466 | −2.549 | 1.00 | 0.00 |
| ATOM | 1233 | CG1 | VAL | 84 | −3.695 | −3.570 | −2.635 | 1.00 | 0.00 |
| ATOM | 1234 | CG2 | VAL | 84 | −1.649 | −4.104 | −1.266 | 1.00 | 0.00 |
| ATOM | 1235 | HA | VAL | 84 | −0.362 | −3.945 | −3.635 | 1.00 | 0.00 |
| ATOM | 1236 | HB | VAL | 84 | −1.940 | −2.405 | −2.485 | 1.00 | 0.00 |
| ATOM | 1237 | HG11 | VAL | 84 | −4.074 | −4.571 | −2.428 | 1.00 | 0.00 |
| ATOM | 1238 | HG12 | VAL | 84 | −4.146 | −2.906 | −1.898 | 1.00 | 0.00 |
| ATOM | 1239 | HG13 | VAL | 84 | −3.974 | −3.181 | −3.615 | 1.00 | 0.00 |
| ATOM | 1240 | HG21 | VAL | 84 | −1.999 | −5.135 | −1.223 | 1.00 | 0.00 |
| ATOM | 1241 | HG22 | VAL | 84 | −0.572 | −3.966 | −1.176 | 1.00 | 0.00 |
| ATOM | 1242 | HG23 | VAL | 84 | −2.087 | −3.670 | −0.367 | 1.00 | 0.00 |
| ATOM | 1243 | H | VAL | 84 | −2.632 | −3.270 | −5.425 | 1.00 | 0.00 |
| ATOM | 1244 | N | ALA | 85 | −1.115 | −6.383 | −3.412 | 1.00 | 0.00 |
| ATOM | 1245 | CA | ALA | 85 | −1.012 | −7.823 | −3.726 | 1.00 | 0.00 |
| ATOM | 1246 | C | ALA | 85 | −0.801 | −8.702 | −2.531 | 1.00 | 0.00 |
| ATOM | 1247 | O | ALA | 85 | −0.279 | −8.327 | −1.407 | 1.00 | 0.00 |
| ATOM | 1248 | CB | ALA | 85 | 0.196 | −7.944 | −4.692 | 1.00 | 0.00 |
| ATOM | 1249 | HA | ALA | 85 | −1.930 | −8.089 | −4.250 | 1.00 | 0.00 |
| ATOM | 1250 | OXT | ALA | 85 | −1.159 | −9.943 | −2.647 | 1.00 | 0.00 |
| ATOM | 1251 | HB1 | ALA | 85 | 0.254 | −8.992 | −4.989 | 1.00 | 0.00 |
| ATOM | 1252 | HB2 | ALA | 85 | 0.138 | −7.315 | −5.580 | 1.00 | 0.00 |
| ATOM | 1253 | HB3 | ALA | 85 | 1.099 | −7.691 | −4.137 | 1.00 | 0.00 |
| ATOM | 1254 | H | ALA | 85 | −0.478 | −6.163 | −2.661 | 1.00 | 0.00 |
| TER | 1255 | | ALA | 85 | | | | | |
| ATOM | 1256 | H7 | CPX | 86 | −4.120 | 5.294 | 9.673 | 1.00 | 0.00 |
| ATOM | 1257 | C8 | CPX | 86 | −3.665 | 5.010 | 8.723 | 1.00 | 0.00 |
| ATOM | 1258 | S12 | CPX | 86 | −3.287 | 6.586 | 7.972 | 1.00 | 0.00 |
| ATOM | 1259 | C11 | CPX | 86 | −4.417 | 6.307 | 6.624 | 1.00 | 0.00 |
| ATOM | 1260 | N10 | CPX | 86 | −5.082 | 5.115 | 6.815 | 1.00 | 0.00 |
| ATOM | 1261 | C9 | CPX | 86 | −4.804 | 4.366 | 7.928 | 1.00 | 0.00 |
| ATOM | 1262 | O17 | CPX | 86 | −5.258 | 3.252 | 8.008 | 1.00 | 0.00 |
| ATOM | 1263 | S18 | CPX | 86 | −4.546 | 7.344 | 5.191 | 1.00 | 0.00 |
| ATOM | 1264 | H8 | CPX | 86 | −3.859 | 8.080 | 5.246 | 1.00 | 0.00 |
| ATOM | 1265 | C7 | CPX | 86 | −2.494 | 4.145 | 9.156 | 1.00 | 0.00 |
| ATOM | 1266 | H6 | CPX | 86 | −1.710 | 4.763 | 9.591 | 1.00 | 0.00 |
| ATOM | 1267 | H5 | CPX | 86 | −2.875 | 3.414 | 9.869 | 1.00 | 0.00 |
| ATOM | 1268 | C1 | CPX | 86 | −1.819 | 3.419 | 8.024 | 1.00 | 0.00 |
| ATOM | 1269 | C6 | CPX | 86 | −1.127 | 4.125 | 7.018 | 1.00 | 0.00 |
| ATOM | 1270 | C5 | CPX | 86 | −0.672 | 3.562 | 5.816 | 1.00 | 0.00 |
| ATOM | 1271 | C4 | CPX | 86 | −0.745 | 2.219 | 5.533 | 1.00 | 0.00 |
| ATOM | 1272 | C3 | CPX | 86 | −1.462 | 1.494 | 6.492 | 1.00 | 0.00 |
| ATOM | 1273 | C2 | CPX | 86 | −2.060 | 2.073 | 7.698 | 1.00 | 0.00 |
| ATOM | 1274 | H1 | CPX | 86 | −2.541 | 1.411 | 8.403 | 1.00 | 0.00 |
| ATOM | 1275 | H2 | CPX | 86 | −1.434 | 0.425 | 6.344 | 1.00 | 0.00 |
| ATOM | 1276 | C13 | CPX | 86 | −0.008 | 1.610 | 4.371 | 1.00 | 0.00 |
| ATOM | 1277 | F16 | CPX | 86 | −0.175 | 0.223 | 4.425 | 1.00 | 0.00 |
| ATOM | 1278 | F15 | CPX | 86 | 1.353 | 1.931 | 4.311 | 1.00 | 0.00 |
| ATOM | 1279 | F14 | CPX | 86 | −0.620 | 2.008 | 3.146 | 1.00 | 0.00 |
| ATOM | 1280 | H3 | CPX | 86 | −0.168 | 4.236 | 5.137 | 1.00 | 0.00 |
| ATOM | 1281 | H4 | CPX | 86 | −1.060 | 5.198 | 7.120 | 1.00 | 0.00 |
| TER | 1282 | | CPX | 86 | | | | | |
| HETATM | 1283 | H19 | H19 | 1 | −3.181 | 7.132 | 4.106 | 1.00 | 0.00 |

| | | | | | |
|---|---|---|---|---|---|
| CONECT | 1 | 2 | 15 | 16 | 17 |
| CONECT | 5 | 2 | 6 | 11 | 12 |
| CONECT | 6 | 5 | 7 | 13 | 14 |
| CONECT | 11 | 5 | | | |
| CONECT | 13 | 6 | | | |
| CONECT | 15 | 1 | | | |
| CONECT | 16 | 1 | | | |
| CONECT | 17 | 1 | | | |
| CONECT | 23 | 22 | 25 | 28 | 29 |
| CONECT | 28 | 23 | | | |
| CONECT | 42 | 41 | 44 | 47 | 48 |
| CONECT | 47 | 42 | | | |
| CONECT | 104 | 101 | 105 | 109 | 110 |
| CONECT | 109 | 104 | | | |
| CONECT | 123 | 120 | 124 | 129 | 130 |
| CONECT | 124 | 123 | 125 | 131 | 132 |
| CONECT | 125 | 124 | 126 | 133 | 134 |
| CONECT | 126 | 125 | 127 | 135 | 136 |
| CONECT | 129 | 123 | | | |
| CONECT | 131 | 124 | | | |
| CONECT | 133 | 125 | | | |
| CONECT | 135 | 126 | | | |
| CONECT | 145 | 142 | 146 | 151 | 152 |
| CONECT | 146 | 145 | 147 | 153 | 154 |
| CONECT | 147 | 146 | 148 | 155 | 156 |
| CONECT | 148 | 147 | 149 | 157 | 158 |
| CONECT | 151 | 145 | | | |
| CONECT | 153 | 146 | | | |
| CONECT | 155 | 147 | | | |
| CONECT | 157 | 148 | | | |
| CONECT | 167 | 164 | 168 | 173 | 174 |
| CONECT | 168 | 167 | 169 | 175 | 176 |
| CONECT | 173 | 167 | | | |
| CONECT | 175 | 168 | | | |
| CONECT | 184 | 181 | 185 | 189 | 190 |
| CONECT | 189 | 184 | | | |
| CONECT | 195 | 194 | 196 | 198 | 199 |
| CONECT | 198 | 195 | | | |
| CONECT | 205 | 202 | 206 | 210 | 211 |
| CONECT | 206 | 205 | 207 | 212 | 213 |
| CONECT | 210 | 205 | | | |
| CONECT | 212 | 206 | | | |
| CONECT | 219 | 218 | 220 | 222 | 223 |
| CONECT | 222 | 219 | | | |
| CONECT | 229 | 226 | 230 | 234 | 235 |
| CONECT | 234 | 229 | | | |
| CONECT | 248 | 245 | 249 | 251 | 252 |
| CONECT | 251 | 248 | | | |
| CONECT | 260 | 259 | 262 | 265 | 266 |
| CONECT | 265 | 260 | | | |
| CONECT | 314 | 311 | 315 | 320 | 321 |
| CONECT | 315 | 314 | 316 | 322 | 323 |
| CONECT | 316 | 315 | 317 | 324 | 325 |
| CONECT | 317 | 316 | 318 | 326 | 327 |
| CONECT | 320 | 314 | | | |
| CONECT | 322 | 315 | | | |
| CONECT | 324 | 316 | | | |
| CONECT | 326 | 317 | | | |
| CONECT | 333 | 332 | 334 | 336 | 337 |
| CONECT | 336 | 333 | | | |
| CONECT | 350 | 349 | 351 | 353 | 354 |
| CONECT | 353 | 350 | | | |
| CONECT | 360 | 357 | 361 | 366 | 367 |
| CONECT | 361 | 360 | 362 | 368 | 369 |
| CONECT | 366 | 360 | | | |
| CONECT | 368 | 361 | | | |
| CONECT | 377 | 374 | 378 | 382 | 383 |
| CONECT | 382 | 377 | | | |
| CONECT | 389 | 386 | 390 | 395 | 396 |
| CONECT | 390 | 389 | 391 | 397 | 398 |
| CONECT | 391 | 390 | 392 | 399 | 400 |
| CONECT | 392 | 391 | 393 | 401 | 402 |
| CONECT | 395 | 389 | | | |
| CONECT | 397 | 390 | | | |
| CONECT | 399 | 391 | | | |
| CONECT | 401 | 392 | | | |
| CONECT | 411 | 408 | 412 | 416 | 417 |
| CONECT | 416 | 411 | | | |
| CONECT | 427 | 426 | 428 | 430 | 431 |
| CONECT | 430 | 427 | | | |
| CONECT | 438 | 437 | 440 | 443 | 444 |
| CONECT | 443 | 438 | | | |
| CONECT | 456 | 453 | 457 | 465 | 466 |
| CONECT | 465 | 456 | | | |
| CONECT | 493 | 490 | 494 | 499 | 500 |
| CONECT | 494 | 493 | 495 | 501 | 502 |
| CONECT | 495 | 494 | 496 | 503 | 504 |
| CONECT | 496 | 495 | 497 | 505 | 506 |
| CONECT | 499 | 493 | | | |
| CONECT | 501 | 494 | | | |
| CONECT | 503 | 495 | | | |
| CONECT | 505 | 496 | | | |
| CONECT | 515 | 512 | 516 | 518 | 519 |
| CONECT | 518 | 515 | | | |
| CONECT | 558 | 555 | 559 | 564 | 565 |
| CONECT | 559 | 558 | 560 | 566 | 567 |
| CONECT | 560 | 559 | 561 | 568 | 569 |
| CONECT | 561 | 560 | 562 | 570 | 571 |
| CONECT | 564 | 558 | | | |
| CONECT | 566 | 559 | | | |
| CONECT | 568 | 560 | | | |
| CONECT | 570 | 561 | | | |
| CONECT | 577 | 576 | 578 | 580 | 581 |
| CONECT | 580 | 577 | | | |
| CONECT | 584 | 583 | 585 | 587 | 588 |
| CONECT | 587 | 584 | | | |
| CONECT | 614 | 611 | 615 | 619 | 620 |
| CONECT | 619 | 614 | | | |
| CONECT | 642 | 639 | 643 | 647 | 648 |
| CONECT | 647 | 642 | | | |
| CONECT | 651 | 650 | 652 | 654 | 655 |
| CONECT | 654 | 651 | | | |
| CONECT | 661 | 658 | 662 | 669 | 670 |
| CONECT | 662 | 661 | 663 | 671 | 672 |
| CONECT | 663 | 662 | 664 | 673 | 674 |
| CONECT | 669 | 661 | | | |
| CONECT | 671 | 662 | | | |
| CONECT | 673 | 663 | | | |
| CONECT | 685 | 682 | 686 | 690 | 691 |
| CONECT | 690 | 685 | | | |
| CONECT | 721 | 720 | 722 | 724 | 725 |
| CONECT | 724 | 721 | | | |
| CONECT | 731 | 728 | 732 | 736 | 737 |
| CONECT | 736 | 731 | | | |
| CONECT | 743 | 740 | 744 | 749 | 750 |
| CONECT | 744 | 743 | 745 | 751 | 752 |
| CONECT | 749 | 743 | | | |
| CONECT | 751 | 744 | | | |
| CONECT | 760 | 757 | 761 | 765 | 766 |
| CONECT | 765 | 760 | | | |
| CONECT | 779 | 776 | 780 | 784 | 785 |
| CONECT | 784 | 779 | | | |
| CONECT | 798 | 795 | 799 | 801 | 802 |
| CONECT | 801 | 798 | | | |
| CONECT | 825 | 822 | 826 | 830 | 831 |
| CONECT | 830 | 825 | | | |
| CONECT | 834 | 833 | 835 | 837 | 838 |
| CONECT | 837 | 834 | | | |
| CONECT | 844 | 841 | 845 | 852 | 853 |
| CONECT | 845 | 844 | 846 | 854 | 855 |
| CONECT | 846 | 845 | 847 | 856 | 857 |
| CONECT | 852 | 844 | | | |
| CONECT | 854 | 845 | | | |
| CONECT | 856 | 846 | | | |
| CONECT | 868 | 865 | 869 | 871 | 872 |
| CONECT | 871 | 868 | | | |
| CONECT | 879 | 876 | 880 | 884 | 885 |
| CONECT | 884 | 879 | | | |
| CONECT | 911 | 910 | 912 | 914 | 915 |
| CONECT | 914 | 911 | | | |
| CONECT | 921 | 918 | 922 | 926 | 927 |
| CONECT | 926 | 921 | | | |
| CONECT | 940 | 937 | 941 | 943 | 944 |
| CONECT | 943 | 940 | | | |
| CONECT | 951 | 948 | 952 | 957 | 958 |
| CONECT | 952 | 951 | 953 | 959 | 960 |
| CONECT | 957 | 951 | | | |
| CONECT | 959 | 952 | | | |
| CONECT | 968 | 965 | 969 | 974 | 975 |
| CONECT | 969 | 968 | 970 | 976 | 977 |
| CONECT | 974 | 968 | | | |
| CONECT | 976 | 969 | | | |
| CONECT | 983 | 980 | 984 | 991 | 992 |
| CONECT | 984 | 983 | 985 | 993 | 994 |
| CONECT | 985 | 984 | 986 | 995 | 996 |
| CONECT | 991 | 983 | | | |
| CONECT | 993 | 984 | | | |
| CONECT | 995 | 985 | | | |
| CONECT | 1027 | 1024 | 1028 | 1033 | 1034 |
| CONECT | 1028 | 1027 | 1029 | 1035 | 1036 |
| CONECT | 1033 | 1027 | | | |
| CONECT | 1035 | 1028 | | | |
| CONECT | 1042 | 1039 | 1043 | 1047 | 1048 |
| CONECT | 1047 | 1042 | | | |
| CONECT | 1061 | 1058 | 1062 | 1066 | 1067 |
| CONECT | 1062 | 1061 | 1063 | 1068 | 1069 |
| CONECT | 1066 | 1061 | | | |
| CONECT | 1068 | 1062 | | | |
| CONECT | 1092 | 1089 | 1093 | 1100 | 1101 |
| CONECT | 1093 | 1092 | 1094 | 1102 | 1103 |
| CONECT | 1094 | 1093 | 1095 | 1104 | 1105 |
| CONECT | 1100 | 1092 | | | |
| CONECT | 1102 | 1093 | | | |
| CONECT | 1104 | 1094 | | | |
| CONECT | 1130 | 1127 | 1131 | 1133 | 1134 |
| CONECT | 1133 | 1130 | | | |
| CONECT | 1141 | 1138 | 1142 | 1144 | 1145 |
| CONECT | 1144 | 1141 | | | |
| CONECT | 1198 | 1195 | 1199 | 1203 | 1204 |
| CONECT | 1203 | 1198 | | | |
| CONECT | 1217 | 1214 | 1218 | 1223 | 1224 |
| CONECT | 1218 | 1217 | 1219 | 1225 | 1226 |
| CONECT | 1223 | 1217 | | | |
| CONECT | 1225 | 1218 | | | |
| CONECT | 1256 | 1257 | | | |
| CONECT | 1257 | 1256 | 1258 | 1261 | 1265 |
| CONECT | 1258 | 1257 | 1259 | | |
| CONECT | 1259 | 1258 | 1260 | 1263 | |
| CONECT | 1260 | 1259 | 1261 | | |
| CONECT | 1261 | 1257 | 1260 | 1262 | |
| CONECT | 1262 | 1261 | | | |
| CONECT | 1263 | 1259 | 1264 | | |
| CONECT | 1264 | 1263 | | | |
| CONECT | 1265 | 1257 | 1266 | 1267 | 1268 |
| CONECT | 1266 | 1265 | | | |
| CONECT | 1267 | 1265 | | | |
| CONECT | 1268 | 1265 | 1269 | 1273 | |
| CONECT | 1269 | 1268 | 1270 | 1281 | |
| CONECT | 1270 | 1269 | 1271 | 1280 | |
| CONECT | 1271 | 1270 | 1272 | 1276 | |
| CONECT | 1272 | 1271 | 1273 | 1275 | |
| CONECT | 1273 | 1268 | 1272 | 1274 | |
| CONECT | 1274 | 1273 | | | |
| CONECT | 1275 | 1272 | | | |
| CONECT | 1276 | 1271 | 1277 | 1278 | 1279 |
| CONECT | 1277 | 1276 | | | |
| CONECT | 1278 | 1276 | | | |
| CONECT | 1279 | 1276 | | | |
| CONECT | 1280 | 1270 | | | |
| CONECT | 1281 | 1269 | | | |

| | |
|---|---|
| MASTER | 0 0 0 0 0 0 0 0 1281 2 220 8 |
| END | |

TABLE 2
Apparent Kd values of ligands of the PDZ-domains
of AF6 and Syntrophin.

| | | Kd value |
|---|---|---|
| Compound | Kd value (AF6) | (Syntrophin) |
| [No.] | [μM] | [μM] |
| 41 (FIG. 4) | 419 ± 30 | n.d. |
| 12 (FIG. 2A) | 260 ± 12 | n.d. |
| II (FIG. 5) | 480 ± 33 | 398 ± 32 |
| 7 (FIG. 1B) | 280 ± 22 | 330 ± 17 |
| 1 (FIG. 1A) | 101 ± 15 | 239 ± 12 |

Legend:
n.d. = not determined;

The number behind the ± symbol indicates the statistical standard deviation of a mathematical curve adaptation to the chemical binding equation “protein+ligand=complex”.

1. The use of a compound according to Formula I 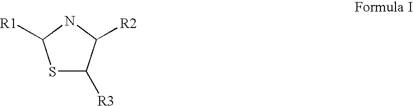
wherein R1 and R2 are ═O or ═S and are identical or different, wherein R2 may alternatively be two —H, wherein R3 is ═CHR4, —CH₂R4 or —CHR5R4,
wherein R4 is phenyl; phenyl simply, doubly or triply substituted with -Hal or —C(Hal)ₙ (n=1, 2, or 3); 2-, 3-, 4-, or 5-thienyl; 2-, 3-, 4-, or 5-thienyl simply, doubly or triply substituted with -Hal or —C(Hal)n (n=1, 2, or 3); 2-, 3-, 4-, or 5-furyl; 2-, 3-, 4-, or 5-furyl simply, doubly or triply substituted with -Hal or —C(Hal)ₙ (n=1, 2, or 3); or C1-C5 alkyl, linear or branched,
wherein -Hal is —F, —Cl, —Br, or -J,
wherein free valences of the ring are bound with hydrogen,
wherein R5 is C1-C5 alkyl, linear or branched, or —CH₂—CO—N(R6)₂ with R6=C1-C5 alkyl, linear or branched,
wherein the ring —S— may be replaced by —O—, —CH₂— or —CO—,
or a physiologically well-tolerated salt of such a compound
for preparing a pharmaceutical composition for modulation of a protein containing a PDZ-domain.

2. The use according to claim 1, wherein R1 is ═S, wherein R2 is ═O, wherein R3 is ═CH—R4.

3. The use according to claim 2, wherein R4 is 3-thienyl, phenyl, or phenyl substituted in para with -Hal, preferably —Br.

4. The use according to claim 1, wherein R1 is ═S, wherein R2 is ═O, wherein R3 is —CH₂—R4, and R4 is 3-furyl or phenyl substituted in para with —CHal₃, preferably —CF₃.

5. The use according to claim 1, wherein R1 is ═O, wherein R2 is ═O, wherein R3 is ═CH—R4.

6. The use according to claim 5, wherein R4 is isopropyl or phenyl substituted in meta or para with —CHal₃, preferably —CF₃.

7. The use according to claim 1, wherein R1 is ═O, wherein R2 is ═O, wherein R3 is —CH₂—R4, and wherein R4 is phenyl substituted in para with -Hal, in particular —Br, or with —CHal₃, in particular —CF₃.

8. The use of a compound according to Formula II 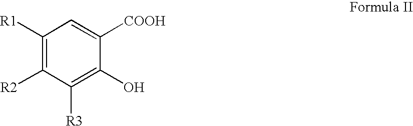
wherein R1 is -Hal or C1-C5 alkyl, linear or branched,
wherein R2 is —H, and
wherein R3 is —NO₂, -Hal, C1-C5 alkyl, linear or branched,
wherein instead of R1 and R2 a ring with —CH═CHal-CH═CH— may be formed, or
wherein instead of R2 and R3 a ring with —CH═CH—CH═CH— may be formed,
wherein -Hal is —F, —Cl, —Br, or -J,
wherein free valences of the ring are bound with hydrogen,
or a physiologically well-tolerated salt of such a compound
for preparing a pharmaceutical composition for modulation of a protein containing a PDZ-domain.

9. The use according to claim 8, wherein R3 is —Br or tertiary-butyl.

10. The use according to claim 8 or 9, wherein R1 is —Cl, —Br, or tertiary-butyl.

11. The use according to claim 1, wherein R1 is —Br and wherein instead of R2 and R3 a ring with —CH═CH—CH═CH— is formed.

12. The use according to claim 1, wherein R3 is —Br and wherein instead of R1 and R2 a ring with —CH═CBr—CH═CH— is formed.

13. The use of a compound according to Formula III 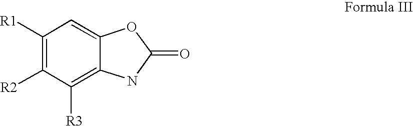
wherein R1 is —NO₂ or -Hal,
wherein R2 and R3 are identical or different and are —H or -Hal,
wherein -Hal is —F, —Cl, —Br, or -J,
wherein free valences of the rings are bound with hydrogen,
or a physiologically well-tolerated salt of such a compound
for preparing a pharmaceutical composition for modulation of a protein containing a PDZ-domain.

14. The use according to claim 13, wherein R1, R2 and R3 are -Hal, in particular —Cl.

15. The use according to claim 13, wherein R1 is —NO₂ and wherein R2 and R3 are —H.

16. A compound according to Formula I 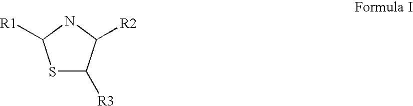
wherein R1 and R2 are ═O or ═S and are identical or different, wherein R2 may alternatively be two —H,
wherein R3 is ═CHR4, —CH₂R4 or —CHR5R4,
wherein R4 is phenyl; phenyl simply, doubly or triply substituted with -Hal or —C(Hal)ₙ (n=1, 2, or 3); 2-, 3-, 4-, or 5-thienyl; 2-, 3-, 4-, or 5-thienyl simply, doubly or triply substituted with -Hal or —C(Hal)n (n=1, 2, or 3); 2-, 3-, 4-, or 5-furyl; 2-, 3-, 4-, or 5-furyl simply, doubly or triply substituted with -Hal or —C(Hal)ₙ (n=1, 2, or 3); or C1-C5 alkyl, linear or branched,
wherein -Hal is —F, —Cl, —Br, or -J,
wherein R5 is C1-C5 alkyl, linear or branched, or —CH₂—CO—N(R6)₂ with R6=C1-C5 alkyl, linear or branched,
wherein free valences of the ring are bound with hydrogen,
wherein R4 is not 2-furyl or 2-thienyl, if R1 is ═O or ═S, and if R3 is ═CH—R4,
wherein R4 is not phenyl or phenyl substituted in meta or para with —CF₃ or —Br, if R1 is ═O or ═S, if R2 is ═O, and if R3 is ═CH—R4 or —CH₂—R4,
wherein R4 is not phenyl, if R1 is ═O or ═S, if R2 is ═O, and if R3 is ═CH—R4,
wherein R4 is not phenyl substituted in para with —Br, if R1 and R2 are ═O, and if R3 is —CH₂—R4,
wherein R4 is not isopropyl, if R1 is ═S, if R2 is ═O, and if R3 is ═CH—R4,
wherein the ring —S— may be replaced by —O—, —CH₂— or —CO—,
or a physiologically well-tolerated salt of such a compound.

17. The compound according to claim 16, wherein R1 is ═S, wherein R2 is ═O, wherein R3 is ═CH—R4.

18. The compound according to claim 17, wherein R4 is 3-thienyl, phenyl, or phenyl substituted in para with -Hal, preferably —Br.

19. The compound according to claim 16, wherein R1 is ═S, wherein R2 is ═O, wherein R3 is —CH₂—R4, and R4 is 3-furyl or phenyl substituted in para with —CHal₃, preferably —CF₃.

20. The compound according to claim 16, wherein R1 is ═O, wherein R2 is ═O, wherein R3 is ═CH—R4.

21. The compound according to claim 20, wherein R4 is isopropyl or phenyl substituted in meta or para with —CHal₃, preferably —CF₃.

22. The compound according to claim 16, wherein R1 is ═O, wherein R2 is ═O, wherein R3 is —CH₂—R4, and wherein R4 is phenyl substituted in para with -Hal, in particular —Br, or with —CHal₃, in particular —CF₃.

23. A method for identification of a modulator of a protein containing a PDZ-domain, wherein a structural model of a modulator candidate is first compared with a structural model of a reference compound, which binds to the PDZ-domain, and is pre-selected in case of an overlap of bioisosteric atoms of the modulator candidate with those of the reference compound, and wherein then the pre-selected modulator candidate is compared with a structural model of the protein in the conformation of a complex of the protein with the reference compound, and it is investigated, whether the modulator candidate binds to the PDZ-domain, wherein the structural model of the protein or of the complex is derived i) from structural coordinates of the complex, ii) from a fragment of the complex containing a PDZ-domain, or of a homolog to i) or ii).

24. The method according to claim 23, wherein the comparison comprises the combination of the structural models of the modulator candidate with the structural model of the protein, wherein optionally the free binding energy of the binding between modulator candidate and protein is determined, and wherein with low free binding energy a high binding probability is detected.

25. The method according to claim 24, wherein the free binding energy is calculated:
by addition of the free energies of interatomic contacts between the structural model of the modulator candidate and the structural model of the protein, and/or
by determination of the free binding energy between the force field of the modulator candidate and the force field of the protein.

26. The use of structural coordinates i) of the protein AF6 complexed with a reference compound, ii) of a fragment containing a PDZ-domain of AF6 complexed with the reference compound, or iii) of a homolog to i) or ii) for identification of a modulator of a protein containing a PDZ-domain, preferably in a method according to one of claims 23 to 25.

27. The method or the use according to one of claims 23 to 26, wherein the structural coordinates are those of Table I.

28. A machine-readable storage medium containing machine-readable data, which after reading-out and processing by means of a data processing system with a suitable software provide a representation of the structural model of a protein or of a complex according to one of claims 23 to 27.

29. A computer software with a software code for carrying-out the method or the use according to one of claims 23 to 27.

30. A data processing system comprising the computer software according to claim 29 and the machine-readable storage medium according to claim 28.

31. The use according to one of claims 1 to 15, wherein the compound is mixed in a physiologically effective dose with at least one auxiliary or carrier substance.

32. The use of a compound according to one of claims 16 to 22 for preparing a pharmaceutical composition, wherein optionally the compound is mixed in a physiologically effective dose with at least one auxiliary or carrier substance.

33. A complex containing the protein AF6 and a substance bound to the PDZ-domain of the protein according to claim 1.

34. The complex according to claim 33, wherein R1 is ═S, R2 is ═O, R3 is —CH₂—R4 and R4 is phenyl substituted in para with CF₃, in particular compound 1.

35. The use of structural coordinates of the complex according to claim 33 or 34 for screening for substances, which bind to the PDZ-domain of the protein AF6.
